(19) United States
(12) Patent Application Publication
(10) Pub. No.: US 2020/0197392 A1
(43) Pub. Date: Jun. 25, 2020

(54) COMPOSITIONS AND METHODS FOR TREATING TUBEROUS SCLEROSIS COMPLEX

(71) Applicant: THE BRIGHAM & WOMEN'S HOSPITAL, INC., Boston, MA (US)

(72) Inventors: David J. KWIATKOWSKI, Weston, MA (US); Mahsa ZAREI, College Station, TX (US)

(73) Assignee: THE BRIGHAM & WOMEN'S HOSPITAL, INC., Boston, MA (US)

(21) Appl. No.: 16/638,976

(22) PCT Filed: Aug. 15, 2018
(86) PCT No.: PCT/US2018/000130

§ 371 Date: Feb. 13, 2020

Related U.S. Application Data
(60) Provisional application No. 62/545,767, filed on Aug. 15, 2017.

(51) Int. Cl. A61K 31/506; A61K 31/519; A61K 31/454; A61K 31/53; A61P 31/06

(52) U.S. Cl. CPC A61K 31/506; A61K 31/519; A61K 45/06; A61K 31/53; A61P 31/06; A61K 31/454

(57) ABSTRACT

Provided herein are methods of treating tuberous sclerosis complex using inhibitors of cyclin dependent kinase 7 (CDK7) alone or in combination with rapamycin inhibitors.

### FIELD OF THE DISCLOSURE

The present disclosure described herein relates to compositions and methods for the treatment of tuberous sclerosis complex (TSC).

### BACKGROUND

Tuberous sclerosis complex (TSC) is a genetic disease with an autosomal dominant pattern of inheritance in which affected individuals develop numerous non-cancerous growths, primarily in the central nervous system (CNS), kidneys and skin. Tuberous sclerosis complex is also associated with a variety of CNS symptoms in humans include learning disabilities, seizures and autism. At present there is no drug therapy that addresses the underlying causes of TSC, and thus treatment of TSC is restricted to management of symptoms associated with the disease.

CNS phenotypes seen in TSC patients include cortical tubers, subependymal nodules (SENs), and subependymal giant cell astrocytomas (SEGAs). Histopathological studies of tubers have indicated disorganized, hamartomatous regions of cortex with abnormal cell morphology; dysplastic neurons; cytomegaly; heterotropic neurons; aberrant dendritic formations and axonal projections; and astrocytic proliferation.

### SUMMARY

The methods and compositions described herein are based, in part, on the discovery that inhibitors of cyclin dependent kinase 7 (CDK7) can selectively kill TSC1- or TSC2-deficient tumor cells. Thus, provided herein are methods of treating tuberous sclerosis complex using such inhibitors.

In one aspect, described herein is a method for treating tuberous sclerosis complex (TSC) in a subject, the method comprising: administering an inhibitor of cyclin dependent kinase 7 (CDK7) to a subject in need thereof, thereby treating tuberous sclerosis complex in the subject.

In one embodiment of this aspect, the subject is first diagnosed as having TSC using a genetic test or by detecting loss of TSC1 and/or TSC2.

In one embodiment, the CDK7 inhibitor inhibits expression and/or activity of CDK7 by at least 10% compared to the expression and/or activity of CDK7 in the subject prior to treatment.

In another embodiment, the CDK7 inhibitor inhibits cell proliferation or viability preferentially in TSC1 and/or TSC2 deficient cells.

In another embodiment, the CDK7 inhibitor reduces aberrant cell proliferation in the subject.

In another embodiment, the CDK7 inhibitor: (i) induces cellular apoptosis; (ii) increases reactive oxygen species (ROS) levels; (iii) decreases glutathione levels or depletes glutathione; (iv) inhibits benign tumor growth associated with TSC; (v) increases production of mitochondrial reactive oxygen species (mtROS); and/or, (vi) decreases expression of glutathione biosynthesis genes.

In another embodiment, the presence or degree of cellular apoptosis induction is assessed by measuring caspase 3 cleavage or by Annexin V staining.

In another embodiment, the CDK7 inhibitor comprises a small molecule, an antibody or antigen-binding fragment thereof, an RNA interference agent, or an antisense RNA.

In another embodiment, the small molecule comprises THZ1 of Formula I, or a derivative thereof that retains CDK7 inhibition activity:

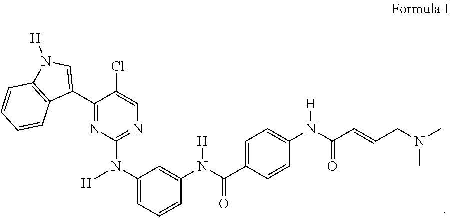 [SMILES: CN(C)C/C=C/C(=O)Nc1ccc(C(=O)Nc2cccc(Nc3ncc(Cl)c(-c4c[nH]c5ccccc45)n3)c2)cc1]

In another embodiment, the THZ1 derivative comprises SY-1365.

In another embodiment, the small molecule inhibitor of CDK7 comprises CT7001 having the formula of Formula II:

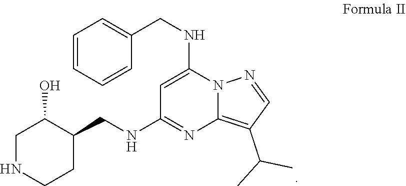 [SMILES: CC(C)c1cnn2c(NCc3ccccc3)cc(NC[C@H]3CCNC[C@@H]3O)nc12]

In another embodiment, the small molecule inhibitor of CDK7 comprises at least one compound selected from the group consisting of compounds 1-186 of Table 1.

In another embodiment, the method further comprises administering at least one additional agent. In another embodiment, the at least one additional agent comprises rapamycin or an analog thereof (a so-called “rapalog”) that retains mTORC1 inhibitory activity. Non-limiting examples of rapalogs include 20-thiarapamycin, 15-deoxo-19-sulfoxylrapamycin, temsirolimus, everolimus, sirolimus, deforolimus, zotarolimus, 42-O-[Morpholinosulfonylcarbamul]-rapamycin, 42-O-[Dimethylaminosulfonylcarbamyl]-rapamycin, 42-O-[N,N-Bis(2-hydroxyethyl)aminosulfonylcarbamyl]-rapamycin, 42-O-[(R)-3-hydroxypyrrolidin-1-ylsulfonylcarbamyl]-rapamycin, 42-O-[4-Hydroxyanilinsulfonylcarbamyl]-rapamycin, 42-O-[4-Methylpiperazine-1-carboxy]-rapamycin, 42-O-[(R)-3-Hydroxypyrrolidin-1-yl)acetyl]-rapamycin, 42-O-[2-(4-Hydroxypiperidin-1-yl)acetyl]-rapamycin, 42-O-[2-(Piperidin-4-yl)ethyl]-rapamycin, 42-O-[3-(4-Methoxycarbonyl-piperidin-1-yl)propyl]-rapamycin, 42-O-[Trimethylsilyl-methyl]-rapamycin, 42-O-[2-(Trimethylsilan-methoxy)-ethyl]-rapamycin, 42-O-[2-(4-(2-Hydroxyethyl)piperidin-1-yl)acetyl]rapamycin, 42-O-[2-(Bis(2-hydroxyethyl)amino)acetyl]-rapamycin, 42-O-(2-Hydroxypiperidincarbonyl)-rapamycin, 42-O-(2-Morpholinoethylaminocarbonyl)-rapamycin, 42-O-[3-(Morpholinosulfonyl)propyl]-rapamycin, or any of the compounds described in WO2017/040341; WO2001/034816; WO2009/131631; or US2011/0098241, the contents of each of which are incorporated herein by reference in their entirety. In another embodiment, the at least one additional agent comprises an inhibitor of mTORC1 (e.g., INK128, AZD8055, AZD2014), or dual mTOR/PI3 kinase inhibitors (e.g., NVP-BEZ235, BGT226, SF1126, or PKI-587).

In another aspect, described herein is a pharmaceutical formulation comprising an amount of a CDK7 inhibitor effective to treat tuberous sclerosis complex in a subject in need thereof, and a pharmaceutically acceptable carrier.

In one embodiment, the CDK7 inhibitor comprises a small molecule, an antibody or antigen-binding fragment thereof, an RNA interference agent, or an antisense RNA.

In another embodiment, the CDK7 inhibitor comprises a molecule selected from: a) THZ1 of Formula I, or a derivative thereof that retains CDK7 inhibition activity; b) CT7001 having the formula of Formula II or a derivative thereof that retains CDK7 inhibition activity and c) a compound selected from the group consisting of compounds 1-186 of Table 1.

In another embodiment, the derivative of THZ1 that retains CDK7 inhibition activity comprises SY-1365.

In another embodiment, the formulation further comprises a therapeutically effective amount of at least one additional therapeutic agent. On another embodiment, the at least one additional therapeutic agent comprises rapamycin or an analog thereof.

In another aspect, described herein is a composition comprising a CDK7 inhibitor for use in the treatment of tuberous sclerosis complex.

In one embodiment, the CDK7 inhibitor is THZ1 or a derivative thereof. In another embodiment, the derivative is SY-1365.

In another embodiment, the composition further comprises at least one additional agent. In another embodiment, the at least one additional agent comprises rapamycin or an analog thereof.

In another embodiment, the composition further comprises a pharmaceutically effective carrier.

In another aspect, also provided herein is a method for reducing growth and/or proliferation in a cell lacking TSC1 and/or TSC2, the method comprising: contacting a cell with an inhibitor of CDK7, thereby reducing the growth and/or proliferation of the cell.

Another aspect described herein relates to a method for increasing apoptosis in a cell lacking TSC1 and/or TSC2, the method comprising: contacting a cell with an inhibitor of CDK7, thereby increasing apoptosis of the cell.

Also provided herein, in another aspect is a combination therapy for TSC, comprising a CDK7 inhibitor and rapamycin (or analog thereof).

### BRIEF DESCRIPTION OF THE DRAWINGS

FIG. 1. THZ1 selectively targets TSC-deficient cells. Indicated TSC1/2/−/− or TSC1/2/+/+ were treated with increasing concentrations of THZ1. Cell viability was assessed after 4 days of treatment using Quant-it PicoGreen dsDNA. Data is represented as mean±SD.

FIGS. 2A-2B. Specific induction of apoptosis by THZ1 in TSC-deficient cells. (FIG. 2A) TSC1-deficient HCV.29 cells or TSC1-expressing HCV.29 cells were treated with vehicle control (DMSO), 30 nM THZ1, 20 nM rapamycin (RAPA), or a combination of both for 72 h. Apoptosis was monitored by flow cytometry using FITC-Annexin V. Each data point represents the mean±SEM of three independent experiments. *** p<0.001 (FIG. 2B) Immunoblot analyses of caspase-3 and actin.

FIG. 3. Growth inhibition of THZ1±rapamycin of HCV29 tumor xenografts. Treatment groups are indicated. Rapamycin 3 mg/kg, 3×/week; THZ1 10 mg/kg, 2×/day. Caliper measurements were used to calculate tumor volume.

FIGS. 4A-4F. THZ1-mediated inhibition of CDK7 leads to selective growth inhibition and apoptosis of TSC mutant cells. (FIG. 4A) Cell growth curves of TSC-null cell lines treated with the indicated doses of THZ1. Cell number was calculated by measurement of dsDNA content using PicoGreen after 5 days in 96-well plate assays. Each data point represents the mean of 4 measurements. SEM are indicated. (FIG. 4B) Phase contrast images of cells that were treated with vehicle control or THZ1 (30 nM) for 4 days. Note THZ1-induced death of TSC1 or TSC2 null cells, but not TSC wild type cells. (FIG. 4C) Images of crystal violet stained cells that were treated with vehicle control or 30 nM THZ1 for 10 days after plating cells. (FIG. 4D) Immunoblot analysis shows that THZ1 inhibits RNAPII CTD phosphorylation in both TSC-null and TSC-addback cells. Cells were treated with vehicle control (first lane) or increasing concentrations of THZ1 (10, 30, 100, 1,000 nM) for 4 hr before lysates were prepared for immunoblotting. (FIG. 4E) Apoptotic cell fraction was counted after treatment with control (CTRL), rapamycin (RAP) (20 nM), THZ1 (30 nM), or a combination of both for 72 hr. Apoptotic cell death was quantified by propidium iodide (PI) staining and flow cytometry, and is shown as the percentage of cells that were PI positive. Each data point represents the mean±SEM of three independent experiments. * p<0.05; ** p<0.01; *** p<0.001. (FIG. 4F) Immunoblot analysis shows that cleaved caspase-3 is increased in total protein lysates from two TSC-null cell lines treated with THZ1(30 nM) with or without rapamycin (Rap) (20 nM) for 72 hr. Beta-actin serves as a loading control.

FIGS. 5A-5D. (FIG. 5A) The table shows IC50 values for THZ1 for different TSC1-null, and TSC2-null cell lines and their addback derivatives. (FIG. 5B) Immunoblot of RNAPolII CTD phosphorylation in total protein lysates from SN-398-TSC2- and SN-398-TSC2-addback cell lines exposed to increasing doses of THZ1 (control, 10, 30, 100, 1,000 nM). Beta-actin serves as a loading control. (FIG. 5C) Apoptotic cells were counted after treatment with control (CTRL), rapamycin (RAP) (20 nM), THZ1 (30 nM), or a combination of both for 72 hr. Apoptotic cells were quantified by propidium iodide (PI) staining and flow cytometry. Each data point represents the mean±SEM of three independent experiments. * p<0.05; ** p<0.01; *** p<0.001. (FIG. 5D) Immunoblot analysis shows that cleaved caspase-3 is increased in total protein lysates from a MEF-Tsc2-null cell line treated with THZ1(30 nM) with or without rapamycin (Rap) (20 nM) for 72 hr, but not in the addback control line. Beta-actin serves as a loading control.

FIGS. 6A-6E. Loss of CDK7 but not CDK12 or CDK13 selectively reduces growth of TSC1 and TSC2 null cells. (FIG. 6A) Immunoblot analysis of cell lines in which CDK7 has been knocked out by either CRISPR/Cas9 (KO, left and middle), or shRNA (right). (FIG. 6B) Dilutional clonal growth assays (top) show reduction in colony growth of TSC1-null or TSC2-null cells with CDK7 loss compared to control and TSC-addback cells, with crystal violet. Quantification of cell growth is shown. Error bars in the bottom panel indicate SEM of triplicate wells from a representative experiment (N.S. non-significant, *** p<0.001). (FIG. 6C) Tumor volume of xenografts derived from HCV.29 cells infected with EV (empty vector), CDK7.KO.1 and CDK7.KO.2 guide RNAs. Cells were infected with lentivirus, selected with puromycin for 2 days, and then harvested for subcutaneous injection. 3 million HCV.29 (viability>94% for all groups, assayed by trypan blue exclusion) were subcutaneously injected in to flanks of nude mice. Each data point represents the mean of tumor volume determined by caliper measurements±SEM (n=5 per group, two tumors per mouse). (FIG. 6D) Phase contrast images of cells infected with virus encoding EV (empty vector), CDK.KO.12 and CDK.KO.13. After infection and selection with puromycin (1.5 mg/ml, 96 hr), cells were seeded in 6-well plates (5,000 cells per well for HCV.29.TSC− and HCV.29.TSC+) and imaged with an inverted microscope. (FIG. 6E) Quantification of relative cell number by PicoGreen assay in cells with KO of CDK7, CDK12, or CDK13, grown for 5 days. Each data point represents the mean of 4 independent experiments±SEM (*** p<0.001).

FIGS. 7A-7D. (FIG. 7A) Relative cell number assessed by measurement of dsDNA content using PicoGreen in 621-101-TSC2- and 621-101-TSC2+ Cells after CDK7 silencing by siRNA. Error bars represent±SEM of triplicate wells from a representative experiment (N.S. non-significant, ** p<0.01). (FIG. 7B) CDK7, CDK12 and CDK13 knockdown efficiency in cells treated with CRISPR/Cas9 constructs targeting human CDK7, CDK12 and CDK13. Left, normalized mRNA levels measured by Q-RT-PCR; right, immunoblot analysis of lysates. Error bars represent±SEM of triplicate wells from a representative experiment. (** p<0.01; *** p<0.001). (FIG. 7C) Q-RT-PCR analysis of CDK7 expression in HCV.29. (EV, CDK7.KO.1 and CDK7.KO.2) xenografts harvested on day 49. Actin was used as normalization control. Each bar represents the mean±SEM (n=5 per group; ** p<0.01; *** p<0.001). (FIG. 7D) Quantification of cell number by measurement of dsDNA content using PicoGreen in 621-101-TSC2- and 621-101-TSC2-addback cells after CDK7, CDK12 and CKD13 knockdown by siRNA. Error bars represent±SEM of triplicate wells from a representative experiment (*** p<0.001).

FIGS. 8A-8G. Reduction of glutathione and increase in ROS in THZ1-treated TSC null cells. (FIG. 8A) Graph of steady-state metabolite levels in 621.101.TSC2− cells in response to THZ1 at 30 nm for 6 hr in comparison to vehicle control. The graph shows the Log 2 fold change for each metabolite. Arrow indicates glutathione. (n=3 samples) (FIG. 8B) Heat map showing the top 15 metabolites with greatest change in 621.101.TSC2− cells, with comparison to control and similarly-treated 621.101.TSC2-addback cells (n=3 samples). The scale is log 2 fold-change. (FIG. 8C) Heat map showing the top 25 metabolites with greatest change in HCV.29.TSC1− and MEF.TSC2− cells treated with THZ1 at 30 nm for 6 hr (n=3 samples) versus control(CTRL)(n=3 samples). (FIG. 8D) Normalized ROS levels in TSC-null and TSC-addback cells treated with control(CTRL), rapamycin(RAP) (20 nM), THZ1(30 nM), or the combination for 48 hr. Each data point represents the mean±SEM of three independent experiments (** p<0.01; *** p<0.001). (FIG. 8E) Normalized ROS levels in TSC-null and TSC-addback cells treated with control(CTRL), THZ1(30 nM), n-acetylcysteine (NAC)(2 mM) or the combination for 48 hr. Each data point represents the mean±SEM of three independent experiments (* p<0.05; ** p<0.01; *** p<0.001). (FIG. 8F) HCV.29.TSC1− and 621-101.TSC2− cells were treated with DMSO (vehicle), THZ1(30 nM), GSH-MEE (2 mM), or the combination for 48 hr. Phase contrast images are at top. Cell death (%) is shown at bottom for these treatments as well as RAP (rapamycin 20 nM) and NAC (2 mM) for HCV.29.TSC1− and 621-101.TSC2− after 48 hours of treatment, measured by Trypan blue staining. Each data point represents the mean±SEM of three independent experiments (* p<0.05; ** p<0.01; *** p<0.001). (FIG. 8G) Confocal microscopic images of HCV.29.TSC1− cells showing localization of ROS, by staining with MitoSOX (5 mM) and Mitotracker Green (200 nM) in cells treated with DMSO(vehicle control), rapamycin(RAP) (20 nM), THZ1(30 nM), or the combination.

FIGS. 9A-9C. (FIG. 9A) Box plots of normalized GSH levels of 621.101.TSC2−, HCV.29.TSC1− and MEF.TSC2-TSC cells treated with vehicle (CTRL) or THZ1(30 nm) for 6 hrs. (n=3 samples,*** p<0.0005). (FIG. 9B) Normalized ROS levels in 97.1.TSC1− and 97.1.TSC1-addback cells treated with control(CTRL), rapamycin(RAP) (20 nM), THZ1(30 nM), or the combination for 48 hr. Each data point represents the mean±SEM of three independent experiments (** p<0.01; *** p<0.001). (FIG. 9C) Cell death of MEF.TSC2− after 48 hours of treatment with indicated compounds was measured via Trypan blue staining assay. Each data point represents the mean±SEM of three independent experiments (* p<0.05; *** p<0.001).

FIGS. 10A-10F. NFE2L2 and glutathione synthetic genes are reduced in expression in TSC null cells in response to THZ1 treatment. (FIG. 10A) mRNA levels assessed by RNA-Seq are shown for HCV.29.TSC1− in comparison to HCV.29.TSC1-addback cells treated with THZ1 at 30 nm for 6 hr. The majority of transcripts are reduced in expression; arrow indicates NFE2L2 (NRF2). Average of two independent samples assessed by RNA-Seq. (FIG. 10B) NRF2, assessed by immunohistochemistry, is shown in normal kidney and angiomyolipoma tumor with loss of TSC2. Nuclear localization of NRF2 was observed (data not shown). (FIG. 10C) Q-PCR-ChIP analysis of H3K27Ac in 621.101.TSC2- and 621.101.TSC2+ cells shows an increase in H3K27Ac marks in the NRF2 promoter in 621.101.TSC2-cells. Results are expressed as the fold enrichment over input. Error bars represent the mean±SEM of three independent experiments, * p<0.005. (FIG. 10D) Relative mRNA expression of the indicated genes in HCV.29.TSC1− cells treated with vehicle (CTRL), or 30 nM THZ1 for the indicated periods of time. Gene expression is normalized to Actin expression. Mean±SD is shown. (FIG. 10E) Immunoblot analysis shows levels of 4 proteins in HCV.29.TSC1− cells treated with 30 nM THZ1 for varying periods of time. Beta-actin serves as a loading control. (FIG. 10F) Phase contrast images (left) of HCV.29.TSC1− and HCV.29.TSC1-addback cells transfected with control siRNA (si.CTRL) or siRNA against NRF2 (si.NRF2) after 3 days. PicoGreen cell number assay at 5 days in HCV.29.TSC− and HCV.29.TSC+ cells after NRF2 silencing, along with siRNA controls (Right). Each data point represents the mean±SEM of three independent experiments (N.S. non-significant, *** p<0.001).

FIGS. 11A-11F. (FIG. 11A) Gene set enrichment analysis of genes with significant changes in expression in THZ-treated HCV.29.TSC1− in comparison to THZ1-treated HCV.29.TSC1+ using Gene Ontology (GO). The top enriched molecular function GO categories are shown. Individual bars represent the Bonferroni-corrected p value for enrichment of specific gene ontology subsets. Values for metabolomic-specific, THZ1-sensitive genes are shown. (FIG. 11B) ChIP analysis of H3K27Ac in HCV.29.TSC1− and HCV.29.TSC1-+ cells. qPCR was performed on immunoprecipitated DNA using primers that amplify NRF2 promoter and intron to verify enrichment of regulatory regions of the NRF2 gene. Results are expressed as the fold enrichment over input. (FIG. 11C) Quantitative PCR to detect expression of indicated gene transcripts in DMSO-treated (CTRL) and 30 nM THZ1-treated 621.101.TSC2− cells under the indicated conditions. Gene expression is normalized to Actin expression and then to control. Data are mean±SD. **p<0.01, ***p<0.001. (FIG. 11D) Immunoblot analysis of DMSO-treated and 30 nM THZ1-treated 621.101.TSC2− cells for various times. Beta-actin serves as a loading control. (FIG. 11E) Immunoblot 48 h after transfection of siRNA against NRF2 shows marked reduction HCV.29-TSC1− cells. (FIG. 11F) Representative plots of cell proliferation measured following treatment with THZ1 or ML385 (an NRF inhibitor).

FIGS. 12A-12F. Effects of CDK7 inhibition with THZ1 on kidney tumor development in Tsc2+/− mice. (FIG. 12A) Experimental plan. Tsc2+/− A/J strain mice develop kidney cystadenomas with 100% penetrance by 4 months of age with progressive tumor development. Tsc2+/− mice were randomized at 5.5 months to vehicle (DMSO), THZ1 (10 mg/kg intraperitoneal two times per day), or rapamycin (3 mg/kg intraperitoneal 3 days per week). (FIG. 12B) Number of tumors per kidney in each treatment group (n=kidney number). ***p<0.001. (FIG. 12C) Tumor volume per kidney, with each data point corresponding to one kidney. (FIG. 12D) Renal cystadenoma histology in the treated mice. Representative tumor images are shown for each treatment cohort. Cystadenomas and tumors each are shown at 100×. The cystadenomas shown are from mice treated with vehicle (CTRL), rapamycin (Rap), or THZ1 for one month. (FIG. 12E) Ki-67 staining to assess cell proliferation in kidney sections from the treated mice. All images are at 100× magnification. Percentage of tumor cells with nuclear immunoreactivity of Ki-67 was scored from six random fields per section. ***p<0.001. (FIG. 12F) NRF2 expression by IHC in Tsc2+/− mouse kidney tumors from control and THZ1-treated mice.

FIGS. 13A-13B. (FIG. 13A) Average body weight of Tsc2+/− A/J strain mice in each treatment group. (FIG. 13B) Intracellular GSH levels were measured in kidney tumors of Tsc2+/− A/J strain mice 16 hr after the final treatment with DMSO or THZ1 (n=5). Data are represented as mean±SD. **p<0.009.

FIGS. 14A-14D. Effects of CDK7 inhibition with THZ1 on xenograft tumor development using HCV-29 cells, and model of effect of CDK7 inhibition. (FIG. 14A) HCV.29-TSC1− xenograft mice were treated with vehicle (CTRL), rapamycin (RAP, 3 mg/kg 3 times per week), THZ1 (10 mg/kg 2 times per day), or combined rapamycin and THZ1, starting 5 weeks after HCV.29 cell injection, when tumors reached to 100 mm3 in size for 30 days. Tumor size was measured every 3rd day using a digital caliper. (FIG. 14B) Cell proliferation was markedly reduced in mice treated with rapamycin, THZ1, or both, in comparison to control, as assessed by nuclear staining using Ki-67. This was quantified by counting four to six random fields per section. Scale bar=50 μm. **p<0.01, ***p<0.001. (FIG. 14C) Apoptotic cell death was increased in tumors from mice treated with THZ1, or combined rapamycin-THZ1, in comparison to vehicle or rapamycin treatment. This was quantified by counting four to six random fields per section. Scale bar 50 μm. not shown (n=6) ***p<0.001. (FIG. 14D) Diagram showing glutathione synthetic pathway and ROS generation in TSC mutant cells. Top, TSC-deficient cells have hyperactive mTORC1, leading to increased ROS, NRF2 induction, and an increase in transcription of glutathione synthetic genes to yield more glutathione to buffer the increased ROS. Bottom, THZ1 inhibits transcription by covalently binding to CDK7, blocking RNAPolII phosphorylation, leading to marked reduction in NRF2 and downstream gene expression, depleting glutathione stores, and leading to apoptotic cell death.

FIGS. 15A-15C. (FIG. 15A) Average body weight of TSC1-deficient HCV.29 xenograft mice in each treatment group. (FIG. 15B) Representative images of in vivo and excised xenograft tumors of TSC1-deficient HCV.29 cells from mice treated with vehicle (CTRL), Rapamycin (RAP), THZ1, or the combination, at the termination of the experiment (day 64). (FIG. 15C) Tumor volume in rapamycin, THZ1, or combination treated mice in the 60 days following treatment cessation. (n=4 tumors per group). ***p<0.001.

FIG. 16. Effect of SY-1365 on tumor volume in a TSC mouse model. SY-1365 was administered by tail vein injection 2×/week at 40 mg/kg for 4 weeks. Mice were then sacrificed and tumor assessment performed based on histology. These data show a 99% reduction in tumor volume assessed semi-quantitatively.

### DETAILED DESCRIPTION

Tuberous sclerosis complex (TSC) is caused by germline loss-of-function mutations in TSC1 or TSC2. Bi-allelic loss of either TSC1 or TSC2 occurs in TSC tumors, leading to inactivation of the TSC1/TSC2 protein complex, and activation of mTORC1 with multiple downstream effects on anabolism and cell growth. Rapalogs, mTORC1 inhibitors, are effective cytostatic agents for the treatment of TSC, but lifelong therapy appears to be required for continuing benefit.

The technology described herein is based, in part, on the discovery that the growth and survival of TSC-deficient cells are much more sensitive to inhibitors of the cell cycle regulator CDK7 than cells with TSC activity. Thus, TSC tumors, which lack an active TSC1/TSC2 protein complex, can be selectively treated with CDK inhibitors. Further, the data described herein show, in part, that the CDK7 inhibitor THZ1 in combination with rapamycin produces a synergistic effect on reducing the growth and/or proliferation of cells lacking TSC1 and/or TSC2/The following description and examples provide considerations for one of skill in the art to practice the technology described.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the sale meaning as commonly understood by one of ordinary skill in the art to which this invention belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of tuberous sclerosis complex (TSC) or a condition associated with TSC, e.g., presence of benign tumors. The term “treating” includes reducing or alleviating at least one adverse effect or symptom of tuberous sclerosis complex (e.g., size and number of hamartomas, rhabdomyomas, CNS disturbances etc.). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), reduction in hospital admissions or lengths of stay, and/or decreased mortality, whether detectable or undetectable. The term “treatment” of a disease also includes providing relief from the symptoms or side-effects of the disease (including palliative treatment).

As used herein, the term “administering,” refers to the placement of a therapeutic or pharmaceutical composition (e.g., a CDK7 inhibitor) as disclosed herein into a subject by a method or route which results in at least partial delivery of the agent to the desired organ, tissue, or site (e.g., tumor site) in a subject. Pharmaceutical compositions comprising agents as disclosed herein can be administered by any appropriate route which results in an effective treatment in the subject.

The terms “statistically significant” or “significantly” refer to statistical significance and generally mean a two standard deviation (2SD) or greater difference relative to a reference value.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including TSC. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at risk of developing that condition.

As used herein, the term “aberrant cell proliferation” refers to proliferation of cells with a loss of TSC1 and/or TSC2 expression that results in tumor formation, including the benign tumor formation that is characteristic of tuberous sclerosis complex.

As used herein, the term “inhibits cell proliferation or viability preferentially in TSC1 and/or TSC2 deficient cells” means that a lower concentration of an agent, such as a CDK7 inhibitor, is required to reduce cell proliferation or cell viability in a cell lacking active TSC1/TSC2 complex than in a cell that has active TSC1/TSC2 complex. By “reduce cell proliferation or cell viability” in this context is meant at least a 20%, 30%, 40%, 50%, 60%, 70%, 80%, 90% or greater reduction in the rate of cell proliferation, or at least a 20%, 30%, 40%, 50%, 60%, 70%, 80%, 90% or greater increase in cell death, in the presence of a given agent. By “lower concentration” in this context is meant that the concentration of an agent required to reduce the rate of cell proliferation or cell viability by at least 20%, 30%, 40%, 50%, 60%, 70%, 80%, 90% or more is at least 20%, 30%, 40%, 50%, 60%, 70%, 80%, 90% or more lower in a cell lacking active TSC1/TSC2 complex. In some embodiments, the differential between effect on cells with active TSC1/TSC2 complex and cells without active complex is at least 10-fold, at least 20-fold, at least 50-fold, at least 100-fold or more.

As used herein, the term “pharmaceutical composition” refers to the active agent in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “reference level” can refer to a normal, otherwise unaffected cell population or tissue (e.g., a biological sample obtained from a healthy subject, or a biological sample obtained from the subject at a prior time point, or a biological sample that has not yet been contacted with an agent as described herein).

As used herein, an “appropriate negative control” refers to an untreated, substantially identical cell or population (e.g., a patient or the subject to be treated who was not administered an agent described herein, as compared to a non-control cell).

As used herein, an “appropriate positive control” refers to a substantially similar cell or population that has been treated with a therapeutically effective amount of one or more agents (e.g., a CDK7 inhibitor±rapamycin) as described herein. A positive control can be identified by a measurable reduction in e.g., CDK7 expression and/or activity, partial or complete loss of cell viability, reduced proliferation rate, or activation of apoptotic pathways (e.g., detection of cleaved caspase 3 or Annexin V).

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

### Selected Chemical Definitions

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl. Aliphatic groups may be optionally substituted, e.g., as described herein.

The term “alkyl,” as used herein, refers to a monovalent saturated, straight- or branched-chain hydrocarbon such as a straight or branched group of 1-12, 1-10, or 1-6 carbon atoms, referred to herein as C1-C12 alkyl, C1-C10 alkyl, and C1-C6 alkyl, respectively. Alkyl groups may be optionally substituted, e.g., as described herein. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” are art-recognized and refer to unsaturated aliphatic groups analogous in length and possible substitution to the alkyls described above, but that contain at least one double or triple bond, respectively. Exemplary alkenyl groups include, but are not limited to, —CH═CH2 and —CH2CH═CH2.

The term “alkylene” refers to the diradical of an alkyl group.

The terms “alkenylene” and “alkynylene” refer to the diradicals of an alkenyl and an alkynyl group, respectively.

The term “methylene unit” refers to a divalent —CH2— group present in an alkyl, alkenyl, alkynyl, alkylene, alkenylene, or alkynylene moiety.

The term “carbocyclic ring system”, as used herein, means a monocyclic, or fused, spiro-fused, and/or bridged bicyclic or polycyclic hydrocarbon ring system, wherein each ring is either completely saturated or contains one or more units of unsaturation, but where no ring is aromatic.

The term “carbocyclyl” refers to a radical of a carbocyclic ring system. Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like). A carbocyclyl may be optionally substituted.

The term “aromatic ring system” is art-recognized and refers to a monocyclic, bicyclic or polycyclic hydrocarbon ring system, wherein at least one ring is aromatic.

The term “aryl” refers to a radical of an aromatic ring system. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like. An aryl may be optionally substituted, e.g., as described herein.

The term “heteroaromatic ring system” is art-recognized and refers to monocyclic, bicyclic or polycyclic ring system wherein at least one ring is both aromatic and comprises a heteroatom; and wherein no other rings are heterocyclyl (as defined below). In certain instances, a ring which is aromatic and comprises a heteroatom contains 1, 2, 3, or 4 independently selected ring heteroatoms in such ring.

The term “heteroaryl” refers to a radical of a heteroaromatic ring system. Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, 5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S. A heteroaryl may be optionally substituted, e.g., as described herein.

The term “heterocyclic ring system” refers to monocyclic, or fused, spiro-fused, and/or bridged bicyclic and polycyclic ring systems where at least one ring is saturated or partially unsaturated (but not aromatic) and comprises a heteroatom. A heterocyclic ring system can be attached to its pendant group at any heteroatom or carbon atom that results in a stable structure and any of the ring atoms can be optionally substituted.

The term “heterocyclyl” refers to a radical of a heterocyclic ring system. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine, and 1,2,3,4-tetrahydro-2,6-naphthyridine. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S. A heterocyclyl may be optionally substituted.

The term “saturated heterocyclyl” refers to a radical of heterocyclic ring system wherein every ring is saturated, e.g., tetrahydrofuran, tetrahydro-2H-pyran, pyrrolidine, piperidine and piperazine.

“Partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

As described herein, a CDK7 inhibitor contemplated for use in the methods and compositions described herein may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use.

Suitable monovalent substituents on a substitutable carbon atom of an “optionally substituted” group (such as an alkyl, alkenyl, alkynyl, alkylene, alkenylene, alkynylene or the carbon atom of a carbocyclyl, aryl, heterocyclyl or heteroaryl) are independently deuterium; halogen; —(CH2)₀₋₄R^{∘}; —(CH2)₀₋₄OR^{∘}; —O—(CH2)₀₋₄C(O)OR^{∘}; —(CH2)₀₋₄CH(OR^{∘})₂; —(CH₂)₀₋₄SR^{∘}; —(CH₂)₀₋₄Ph (where “Ph” is phenyl), which may be substituted with R^{∘}; —(CH₂)₀₋₄(CH₂)₀₋₁Ph which may be substituted with R^{∘}; —CH═CHPh, which may be substituted with —R^{∘}; —NO₂; —CN; —N₃; —(CH2)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR⁶⁰²₂; —N(R^{∘})C(S)NR^{∘}₂; —(CH₂)_{0- 4}N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄—C(O)—N(R^{∘})—S(O)₂—R^{∘}, —(CH₂)₀₋₄OC(O)R^{∘}; —OC(O)(CH₂)₀₋₄SR^{∘}—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R; —C(NOR^{∘})R^{∘}; —(CH₂)₀₋₄SSR^{∘}; —(CH₂)₀₋₄S(O)₂R^{∘}; —(CH₂)₀₋₄ S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, deuterium, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of Ro, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of Ro together with their intervening atoms), are independently deuterium, halogen, —(CH2)0-2R•, -(haloR•), —(CH2)0-2OH, —(CH2)0-2OR•, —(CH2)0-2CH(OR•)2; —O(haloR•), —CN, —N3, —(CH2)0-2C(O)R•, —(CH2)0-2C(O)OH, —(CH2)0-2C(O)OR•, —(CH2)0-2SR•, —(CH2)0-2SH, —(CH2)0-2NH2, —(CH2)0-2NHR•, —(CH2)0-2NR•2, —NO2, —SiR•3, —OSiR•3, —C(O)SR•, —(C1-4 straight or branched alkylene)C(O)OR•, or —SSR• wherein each R• is unsubstituted or where preceded by “halo” is substituted only with one or more halogens, and is independently selected from C1-4 aliphatic, —CH2Ph, —O(CH2)0-1Ph, or a 5-6-membered saturated, partially unsaturated, or aryl ring having 0-4 heteroatoms independently selected from nitrogen, oxygen, or sulfur. Suitable divalent substituents on a saturated carbon atom of Ro include ═O and ═S.

Suitable divalent substituents on a saturated carbon atom of an “optionally substituted” group include the following: ═O, ═S, ═NNR*2, ═NNHC(O)R*, ═NNHC(O)OR*, ═NNHS(O)2R*, ═NR*, ═NOR*, —O(C(R*2))2-3O—, or —S(C(R*2))2-3S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)2-3O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include deuterium, halogen, —R•, -(haloR•), —OH, —OR•, —O(haloR•), —CN, —C(O)OH, —C(O)OR•, —NH2, —NHR•, —NR•2, or —NO₂, wherein each R• is unsubstituted or where preceded by “halo” is substituted only with one or more halogens, and is independently C1-4 aliphatic, —CH2Ph, —O(CH2)0-1Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†2, —C(O)R†, —C(O)OR†, —C(O)C(O)R†, —C(O)CH2C(O)R†, —S(O)2R†, —S(O)2NR†2, —C(S)NR†2, —C(NH)NR†2, or —N(R†)S(O)2R†; wherein each R† is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently deuterium, halogen, —R•, -(haloR•), —OH, —OR•, —O(haloR•), —CN, —C(O)OH, —C(O)OR•, —NH2, —NHR•, —NR•2, or —NO₂, wherein each R• is unsubstituted or where preceded by “halo” is substituted only with one or more halogens, and is independently C1-4aliphatic, —CH2Ph, —O(CH2)0-1Ph, or a 5-6-membered saturated, partially unsaturated, or aryl ring having 0-4 heteroatoms independently selected from nitrogen, oxygen, or sulfur.

“Halo” or “halogen” refers to fluorine (fluoro, —F), chlorine (chloro, —Cl), bromine (bromo, —Br), or iodine (iodo, —I).

The term “one or more methylene units of the alkylene, alkenylene or alkynylene is optionally replaced with —O—, —S—, —S(═O)2, or —NRX—” as used herein means that none, one, more than one, or all of the methylene units present may be so replaced. Thus, for example, the moieties, —O—, —S—, and —NRX— are included in this definition because in each case they represent a C1 alkylene (i.e., methylene) replaced with —O—, —S—, or —NRX—, respectively.

It should also be understood that reference to a variable or subvariable e.g., in Formula III (e.g., R2, R3, or R4) being “an optionally substituted C1-C4 alkylene, and an optionally substituted C2-C4 alkenylene or alkynylene, wherein: one or more methylene units of the alkylene, alkenylene or alkynylene other than a methylene unit bound to a nitrogen atom is optionally and independently replaced with —O—, —S—, —N(R6)-, —NHC(O)—, —C(O)NH—, —C(O)—, or —S(═O)2-” is only intended to encompass chemically stable combinations of optionally substitutions and replacements.

Other terms are defined herein within the description of the various aspects of the invention and in the Examples.

### Tuberous Sclerosis Complex (TSC)

Tuberous sclerosis complex (TSC) is a rare genetic disease that causes tumors to form throughout many organ systems in an affected subject, including tumors in the brain, eyes, heart, kidney, skin and lungs. Tumors in the CNS system can cause seizures, developmental delay, cognitive disability, and autism, while tumors in the heart (e.g., cardiac rhabdomyomas) can cause loss of heart function or severe arrhythmia. Renal angiomyolipomas (e.g., kidney tumors associated with TSC) can disrupt normal kidney function if they grow too large. However, the severity of the disease and accompanying symptoms can vary widely among affected individuals.

TSC is inherited in an autosomal dominant fashion, meaning that the disease can be inherited from a single parent having TSC. In addition, TSC can occur through spontaneous genetic mutation, which is responsible for as many as two-thirds of all known TSC cases.

There are currently no treatments available for tuberous sclerosis complex, however intervention with agents that can reduce symptoms associated with TSC can be used to lessen severity and help to manage the disease. Such agents or modalities that can be used to manage TSC symptoms include, but are not limited to, anti-seizure medications, surgery, blood pressure medications, dialysis, organ transplant (e.g., kidney transplant), drugs to shrink tumors (e.g., Afinitor™ (everolimus)), laser treatment, topical ointments (e.g., sirolimus), anti-arrhythmic agents, occupational therapy, physical therapy, speech therapy, and anti-epileptic agents (e.g., vigabatrin), among others.

TSC is typically diagnosed based on a combination of symptoms and genetic testing. Electroencephalogram can be used to aid diagnosis in a subject having seizures, while magnetic resonance imaging, computerized tomography scanning and/or ultrasound can be used to detect growths or tumors in the body (e.g., brain, lungs, kidneys and liver evaluation). Echochardiograms or electrocardiogram can be used to determine if a subject's heart is affected or if cardiac rhabdomyomas are present. Genetic identification of TSC can be determined by detecting the loss of TSC1 and/or TSC2 in cells, for example, of a tumor. Other major diagnostic criteria for TSC are shown in the following table, any one of which can be used in diagnosing TSC, or to monitor treatment efficacy.

| SITE | SYMPTOM | AGE OF ONSET |
|---|---|---|
| Head | Facial angiofibromas or fibrous | Infant to adult |
| | cephalic plaque (at least 3) | |
| Digits | Non-traumatic ungual or | Adolescent |
| (fingers | periungual fibroma (>2) | to adult |
| and toes) | | |
| Skin | Hypomelanotic macules (at | Infant to child |
| | least 3; >5 mm in diameter) | |
| Skin | Shagreen patch (connective | Child |
| | tissue nevus) | |
| Brain | Cortical dysplasias (includes | Fetus |
| | tubers and cerebral white | |
| | matter radial migration lines) | |
| Brain | Subependymal nodule or | Child to |
| | subependymal giant cell | adolescent |
| | astrocytoma | |
| Eyes | Multiple retinal modular | Infant |
| | hamartomas | |
| Heart | Cardiac rhabdomyoma | Fetus |
| Lungs | Lymphangioleiomyomatosis | Adolescent to adult |
| Kidney | Renal angiomyolipoma | Child to adult |

As many as 80% of TSC cases result from mutations in TSC1 and/or TSC2. TSC1 encodes hamartin. TSC2 encodes tuberin, which is thought to interact with, and be stabilized by, hamartin. Overexpression of either TSC1 or TSC2 has growth-suppressing effects (Miloloza et al., 2000; Jin et al., 1996). The gene products of TSC1 and TSC2 form a complex (e.g., hamartin-tuberin complex) and activates the G-protein Ras homologue enriched in brain (Rheb), which in turn inhibits mammalian target of rapamycin complex 1 (mTORC1), a regulator of cell growth.

Thus, in the absence of expression of either the gene product of TSC1 (i.e., hamartin) or that of TSC2 (e.g., tuberin), mTORC1 activity is unchecked and unregulated cell growth and proliferation occurs, which results in the production of benign tumors in afflicted subjects.

### Inhibitors of CDK7

Cyclin-dependent kinase 7 (CDK7) and other cyclin-dependent kinases are involved in the regulation of cell cycle progression. CDK7 is an important component of the transcription factor TFIIH, which is involved in transcription initiation and DNA repair. In addition, CDK7 plays a critical role in regulation of transcription initiation through phosphorylation of the carboxyl-terminal domain (CTD) of RNA Polymerase II (RNAPolII) at multiple sites. CDK7 also controls transcriptional elongation by activating other CDKs (Akhtar et al., 2009; Glover-Cutter et al., 2009; Larochelle et al., 2012; Zhou et al., 2012). Further, CDK7 inhibitors have been postulated for use in the treatment of human glioma (see e.g., Greenall et al. Oncogenesis 6:e336 (2017)).

Provided herein are methods and compositions comprising CDK7 inhibitors that can be used in the treatment of tuberous sclerosis complex and its associated conditions. The various aspects described herein include the administration of one or more therapeutic agents that inhibit CDK7 for the treatment of tuberous sclerosis complex. Also provided herein are methods, compositions and combination therapies comprising a CDK7 inhibitor and rapamycin, which act synergistically to enhance the effect of the CDK7 inhibitor.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of a target polypeptide or a polynucleotide encoding it. Agents that inhibit CDK7, e.g., inhibit CDK7 expression, e.g., translation, post-translational processing, stability, degradation, or nuclear or cytoplasmic localization of a polypeptide, or transcription, post transcriptional processing, stability or degradation of a polynucleotide encoding CDK7 or a polynucleotide encoding a regulator of CDK7 expression or activity, or bind to, partially or totally block stimulation, DNA binding, transcription factor activity or enzymatic activity, or decrease, prevent, or delay activation, or inactivate, desensitize, or down regulate the activity of a polypeptide or polynucleotide. An agent can act directly or indirectly.

An “agent” can be any chemical, entity or moiety, including without limitation synthetic and naturally-occurring proteinaceous and non-proteinaceous entities. In some embodiments, an agent is a nucleic acid, nucleic acid analog, protein, antibody, peptide, aptamer, oligomer of nucleic acids, amino acids, or carbohydrates including without limitation a protein, oligonucleotide, ribozyme, DNAzyme, glycoprotein, siRNAs, lipoprotein and/or a modification or combinations thereof etc. In certain embodiments, agents are small molecule chemical moieties. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertions and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Agents can be known to have a desired activity and/or property, or can be identified from a library of diverse compounds. Methods for screening small molecules are known in the art and can be used to identify a small molecule that is effective at, for example, inhibition of CDK7 activity and/or expression.

Non-limiting examples of small molecule inhibitors of CDK7 include THZ1 (and derivatives thereof), SY-1365, CT7001 (see e.g., Clark et al. Blood 130:245 (2017)), ICEC0942 (see e.g., Patel et al. Molecular Cancer Therapeutics 1-11 (2018)), BAY1000394, flavopiridol (see e.g., Cicenas et al. Cancers 6(4):2224-22242 (2014)), VMY-1-101, VMY-1-103, and BS—181 (Wang et al. Drug Des Develop Ther 10: 1181-1189 (2016)), and CDK7 inhibitors as described in U.S. 2018/0008604.

In some embodiments, the CDK7 inhibitor comprises a compound of Formula III

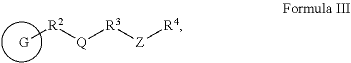

or a pharmaceutically acceptable salt, solvate, hydrate, tautomer or stereoisomer thereof, wherein G is selected from:

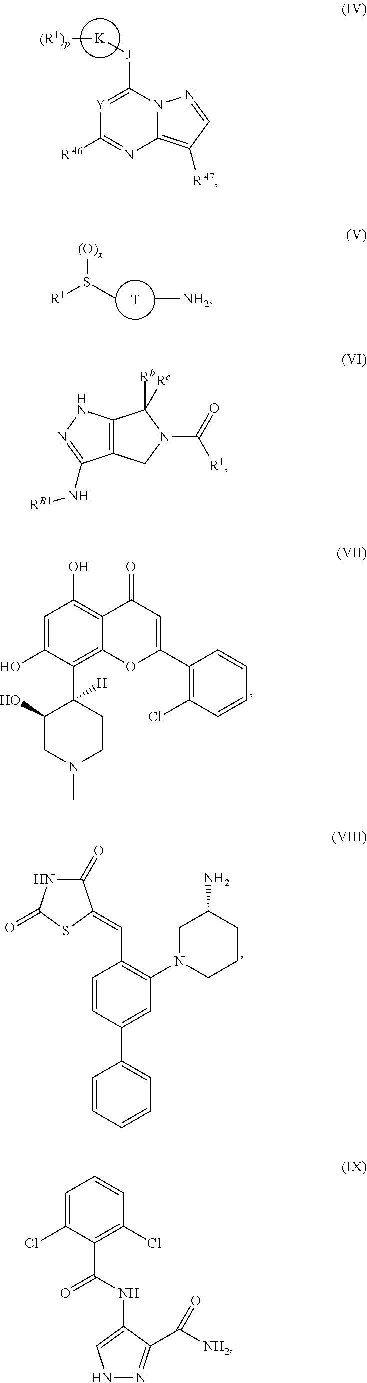

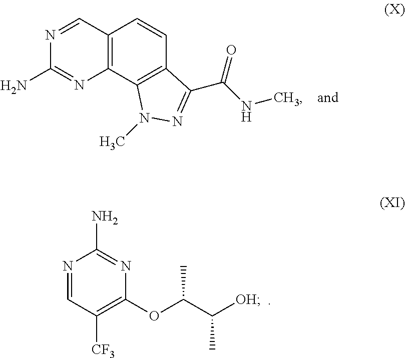 [SMILES: CNC(=O)c1nn(C)c2c1ccc1cnc(N)nc12 ; "and" ; C[C@@H](O)[C@@H](C)Oc1nc(N)ncc1C(F)(F)F]

wherein a hydrogen on G is replaced by a bond to R2, and each R1 is independently selected from hydrogen, halogen, heterocyclyl, aryl, heteroaryl, optionally substituted C1-C6 alkyl, carbocyclyl, —ORa, —NRbRc, —C(O)Ra, —C(O)NRbRc, —S(O)xRa, and —S(O)xNRbRc; RA6 is hydrogen, halogen, heterocyclyl, C1-C6 alkyl, carbocyclyl, —ORa, —NRbRc, —C(O)Ra, —C(O)NRbRc, —S(O)xRa, or —S(O)xNRbRc; RA7 is hydrogen, halogen, heterocyclyl, C1-C6 alkyl, carbocyclyl, —ORa, —NRbRc, —C(O)Ra, —C(O)NRbRc, —S(O)xRa, or —S(O)xNRbRc; each Ra is independently selected from hydrogen, C1-C6 alkyl, optionally substituted aryl, optionally substituted heteroaryl, and optionally substituted heterocyclyl; each Rb and Rc is independently selected from hydrogen and —C1-C6 alkyl, or Rb and Rc taken together with the atom to which they are attached form a 3-7-membered ring; Y is N or CH; K is bond, aryl, heteroaryl, carbocyclyl, or heterocyclyl; J is —NH— or —O—; T is a 5-membered aryl or heteroaryl; p is 0, 1, 2, 3, 4, or 5; x is 0, 1, or 2; R2 is a bond, an optionally substituted C1-C4 alkylene or an optionally substituted C2-C4 alkenylene or alkynylene, wherein one or more methylene units of the alkylene, alkenylene or alkynylene are optionally and independently replaced with —O—, —S—, —C(O)—, or —N(R6)-, wherein R6 is hydrogen or a C1-C6 alkyl chain, and AIk1 is an optionally substituted divalent hydrocarbyl chain containing from 1 to 6 carbon atoms in length and optionally unsaturated bonds between at least two carbon atoms of AIk1 when AIk1 contains at least two carbon atoms; Q is selected from a bond, an optionally substituted divalent carbocyclyl, an optionally substituted divalent heterocyclyl, an optionally substituted divalent aryl, and an optionally substituted divalent heteroaryl; R3 is selected from a bond, an optionally substituted C1-C4 alkylene, and an optionally substituted C2-C4 alkenylene or alkynylene, wherein one or more methylene units of the alkylene, alkenylene or alkynylene is optionally and independently replaced with —O—, —S—, —N(R6)-, —NHC(O)—, —C(O)NH—, —C(O)—, or —S(═O)2-; each R6 is independently selected from hydrogen and optionally substituted —C1-C6 alkyl; Z is selected from a bond; a monocyclic or bicyclic aryl, carbocyclyl, heterocyclyl or heteroaryl, wherein when Z is other than a bond, Z is optionally substituted; R4 is any one of the Formulae (ii-0)-(ii-19):

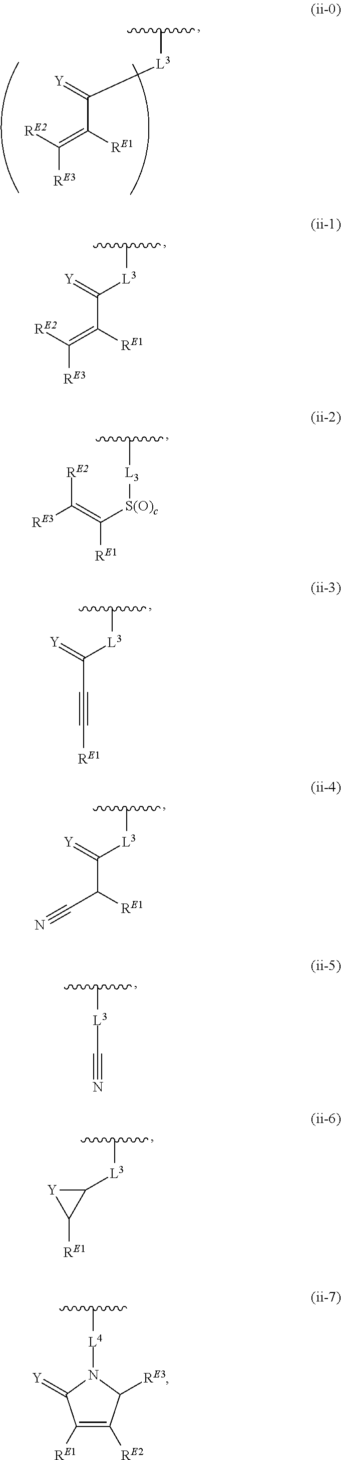

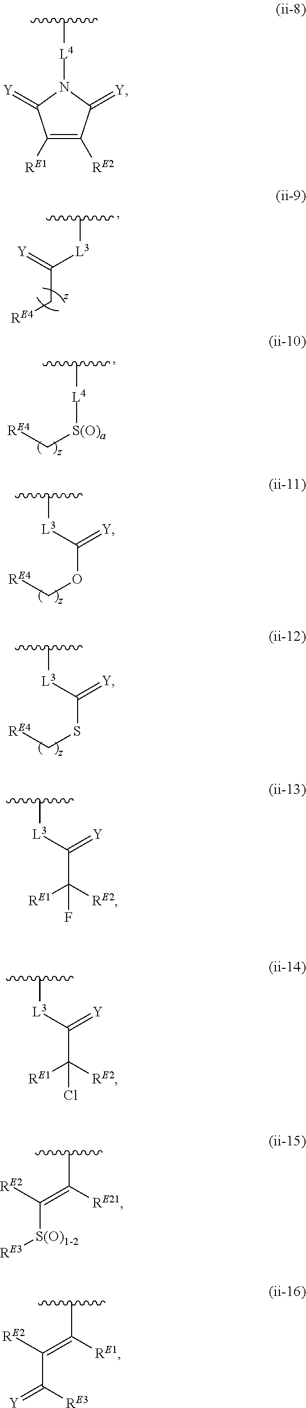

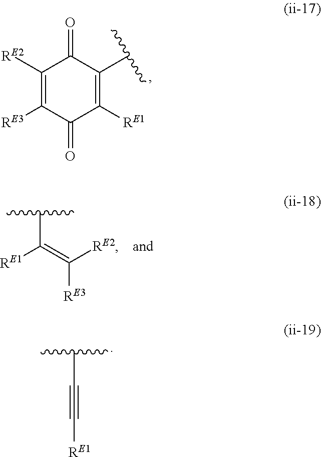

wherein: L3 is a bond, an optionally substituted C1-C7 alkylene, or an optionally substituted C2-C7 alkenylene or alkynylene, wherein one or more methylene units of the alkylene, alkenylene or alkynylene are optionally and independently replaced with —O—, —S—, —S(O)—, —S(O)₂, —N—, or —N(R6)-; L4 is a bond, an optionally substituted C1-C4 alkylene, or an optionally substituted C2-C4 alkenylene or alkynylene; each of RE1, RE2 and RE3 is independently selected from hydrogen, deuterium, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CH2OR9, —CH2N(R9)2, —CH2SR9, —CN, —OR9, —N(R9)2, and —SR9, wherein each occurrence of R9 is independently selected from hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or RE1 and RE3, or RE2 and RE3, or RE1 and RE2 are joined to form an optionally substituted carbocyclic or optionally substituted heterocyclic ring; RE4 is a leaving group; Y is O, S, or N(R6); and z is 0, 1, 2, 3, 4, 5, or 6; when Q is phenyl, Z is other than a bond; and except in the case wherein R4 is (ii-O), no more than one of Q, R3, and Z is a bond.

TABLE 1
Exemplary compounds having CDK7 activity to be used in the methods and compositions described herein.

| | |
|---|---|
| 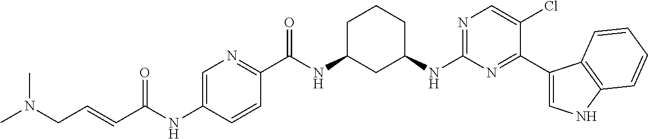 [SMILES: CN(C)C/C=C/C(=O)Nc1ccc(C(=O)N[C@H]2CCC[C@@H](Nc3ncc(Cl)c(-c4c[nH]c5ccccc45)n3)C2)nc1] | Compound 1 |
| 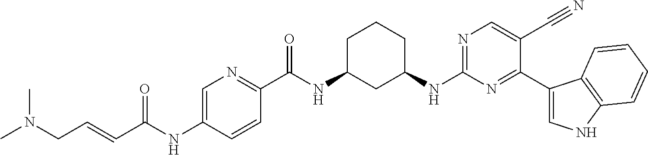 [SMILES: CN(C)C/C=C/C(=O)Nc1ccc(C(=O)N[C@H]2CCC[C@@H](Nc3ncc(C#N)c(-c4c[nH]c5ccccc45)n3)C2)nc1] | Compound 2 |
| 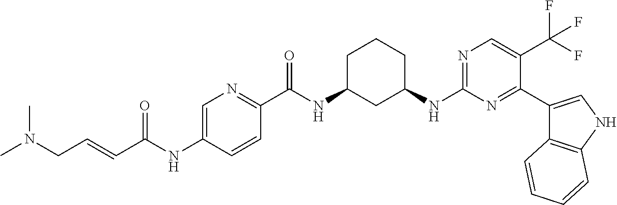 [SMILES: CN(C)C/C=C/C(=O)Nc1ccc(C(=O)N[C@H]2CCC[C@@H](Nc3ncc(C(F)(F)F)c(-c4c[nH]c5ccccc45)n3)C2)nc1] | Compound 3 |
| 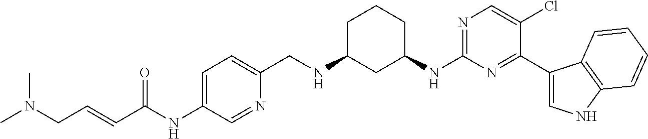 [SMILES: CN(C)C/C=C/C(=O)Nc1ccc(CN[C@H]2CCC[C@@H](Nc3ncc(Cl)c(-c4c[nH]c5ccccc45)n3)C2)nc1] | Compound 4 |
| 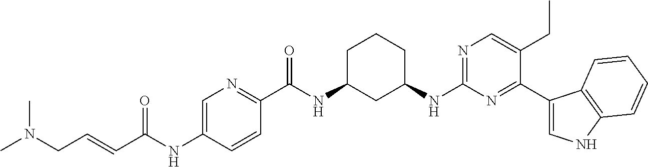 [SMILES: CCc1cnc(N[C@@H]2CCC[C@H](NC(=O)c3ccc(NC(=O)/C=C/CN(C)C)cn3)C2)nc1-c1c[nH]c2ccccc12] | Compound 5 |
| 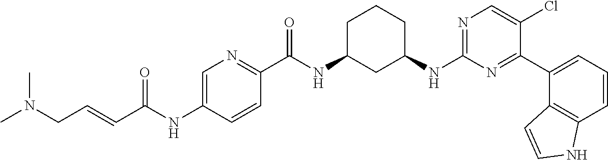 [SMILES: CN(C)C/C=C/C(=O)Nc1ccc(C(=O)N[C@H]2CCC[C@@H](Nc3ncc(Cl)c(-c4cccc5[nH]ccc45)n3)C2)nc1] | Compound 6 |
| 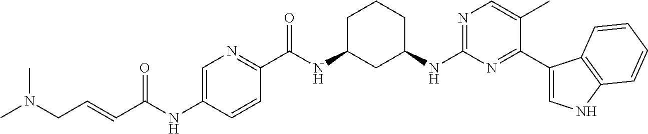 [SMILES: Cc1cnc(N[C@@H]2CCC[C@H](NC(=O)c3ccc(NC(=O)/C=C/CN(C)C)cn3)C2)nc1-c1c[nH]c2ccccc12] | Compound 7 |
| 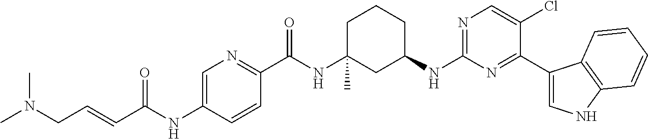 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1] | Compound 8 |
| 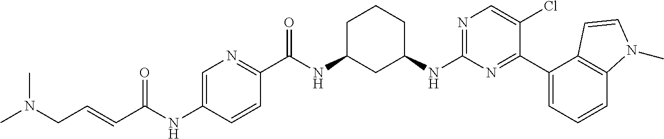 | Compound 9 |
| 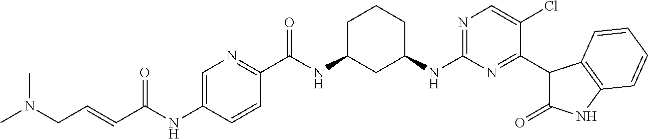 [SMILES: CN(C)C/C=C/C(=O)Nc1ccc(C(=O)N[C@H]2CCC[C@@H](Nc3ncc(Cl)c(C4C(=O)Nc5ccccc54)n3)C2)nc1] | Compound 10 |
| 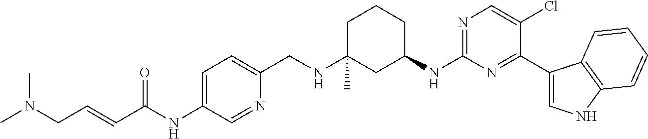 [SMILES: CN(C)C/C=C/C(=O)Nc1ccc(CN[C@@]2(C)CCC[C@@H](Nc3ncc(Cl)c(-c4c[nH]c5ccccc45)n3)C2)nc1] | Compound 11 |
| 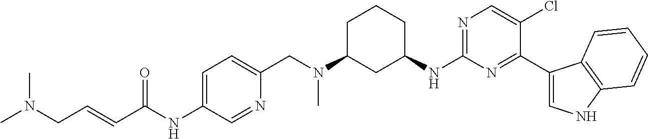 [SMILES: CN(C)C/C=C/C(=O)Nc1ccc(CN(C)[C@H]2CCC[C@@H](Nc3ncc(Cl)c(-c4c[nH]c5ccccc45)n3)C2)nc1] | Compound 12 |
| 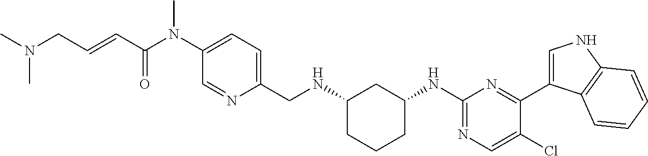 [SMILES: CN(C)C/C=C/C(=O)N(C)c1ccc(CN[C@H]2CCC[C@@H](Nc3ncc(Cl)c(-c4c[nH]c5ccccc45)n3)C2)nc1] | Compound 13 |
| 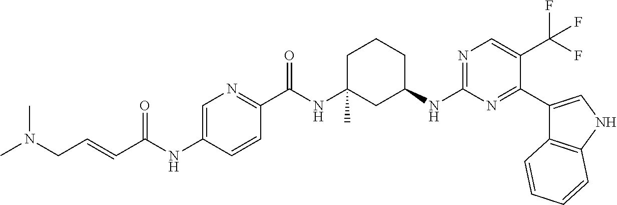 [SMILES: CN(C)C/C=C/C(=O)Nc1ccc(C(=O)N[C@@]2(C)CCC[C@@H](Nc3ncc(C(F)(F)F)c(-c4c[nH]c5ccccc45)n3)C2)nc1] | Compound 14 |
| 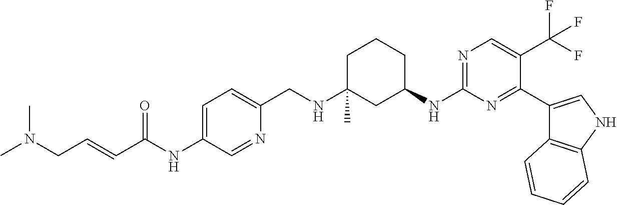 [SMILES: CN(C)C/C=C/C(=O)Nc1ccc(CN[C@@]2(C)CCC[C@@H](Nc3ncc(C(F)(F)F)c(-c4c[nH]c5ccccc45)n3)C2)nc1] | Compound 15 |
| 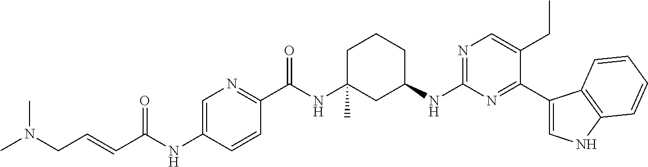 [SMILES: CCc1cnc(N[C@@H]2CCC[C@](C)(NC(=O)c3ccc(NC(=O)/C=C/CN(C)C)cn3)C2)nc1-c1c[nH]c2ccccc12] | Compound 16 |
| 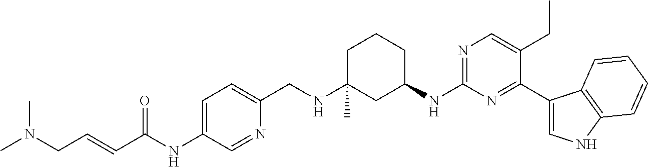 [SMILES: CCc1cnc(N[C@@H]2CCC[C@](C)(NCc3ccc(NC(=O)/C=C/CN(C)C)cn3)C2)nc1-c1c[nH]c2ccccc12] | Compound 17 |
| 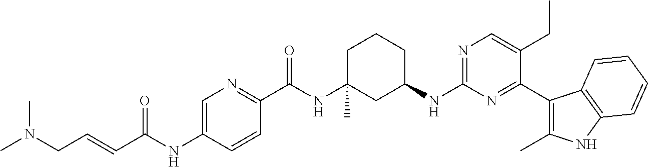 [SMILES: CCc1cnc(N[C@@H]2CCC[C@](C)(NC(=O)c3ccc(NC(=O)/C=C/CN(C)C)cn3)C2)nc1-c1c(C)[nH]c2ccccc12] | Compound 18 |
| 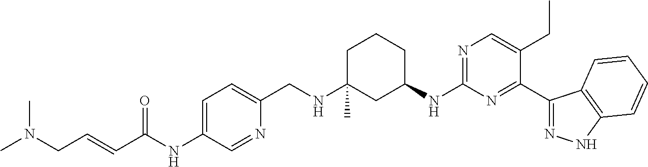 [SMILES: CCc1cnc(N[C@@H]2CCC[C@](C)(NCc3ccc(NC(=O)/C=C/CN(C)C)cn3)C2)nc1-c1n[nH]c2ccccc12] | Compound 19 |
| 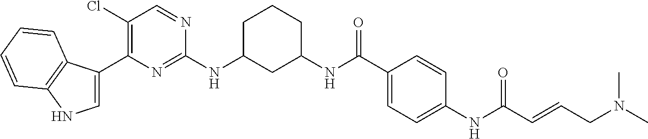 [SMILES: CN(C)C/C=C/C(=O)Nc1ccc(C(=O)NC2CCCC(Nc3ncc(Cl)c(-c4c[nH]c5ccccc45)n3)C2)cc1] | Compound 20 |
| 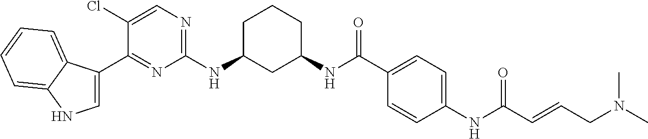 [SMILES: CN(C)C/C=C/C(=O)Nc1ccc(C(=O)N[C@@H]2CCC[C@H](Nc3ncc(Cl)c(-c4c[nH]c5ccccc45)n3)C2)cc1] | Compound 21 |
| 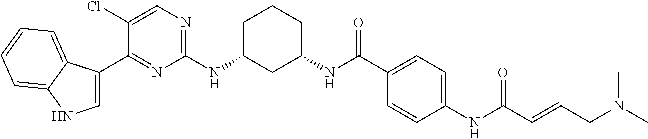 [SMILES: CN(C)C/C=C/C(=O)Nc1ccc(C(=O)N[C@H]2CCC[C@@H](Nc3ncc(Cl)c(-c4c[nH]c5ccccc45)n3)C2)cc1] | Compound 22 |
| 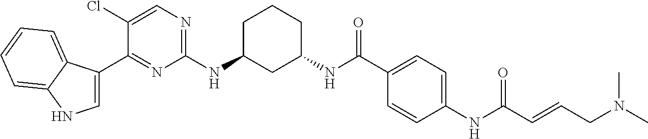 [SMILES: CN(C)C/C=C/C(=O)Nc1ccc(C(=O)N[C@H]2CCC[C@H](Nc3ncc(Cl)c(-c4c[nH]c5ccccc45)n3)C2)cc1] | Compound 23 |
| 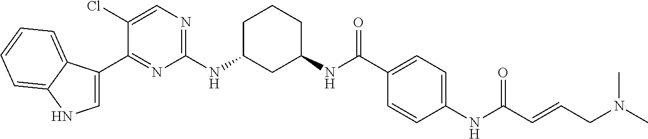 [SMILES: CN(C)C/C=C/C(=O)Nc1ccc(C(=O)N[C@@H]2CCC[C@@H](Nc3ncc(Cl)c(-c4c[nH]c5ccccc45)n3)C2)cc1] | Compound 24 |
| 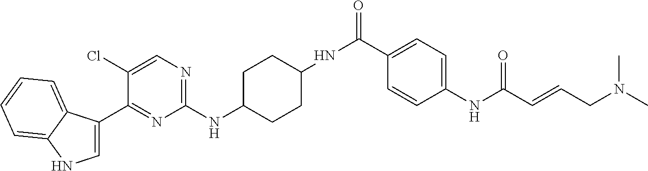 [SMILES: CN(C)C/C=C/C(=O)Nc1ccc(C(=O)NC2CCC(Nc3ncc(Cl)c(-c4c[nH]c5ccccc45)n3)CC2)cc1] | Compound 25 |
| 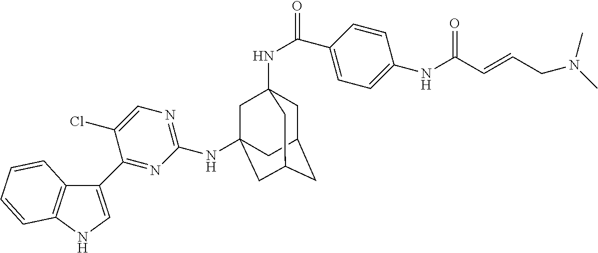 [SMILES: CN(C)C/C=C/C(=O)Nc1ccc(C(=O)NC23CC4CC(C2)CC(Nc2ncc(Cl)c(-c5c[nH]c6ccccc56)n2)(C4)C3)cc1] | Compound 26 |
| 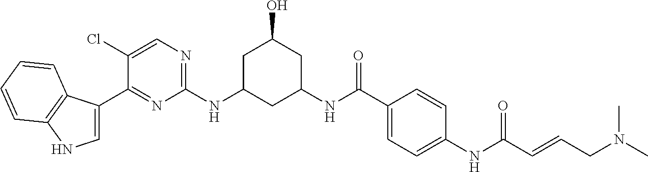 [SMILES: CN(C)C/C=C/C(=O)Nc1ccc(C(=O)NC2CC(Nc3ncc(Cl)c(-c4c[nH]c5ccccc45)n3)C[C@@H](O)C2)cc1] | Compound 27 |
| 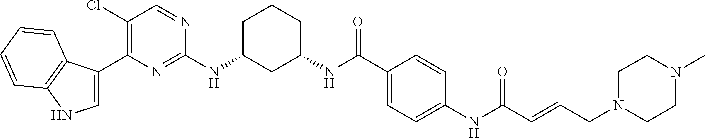 [SMILES: CN1CCN(C/C=C/C(=O)Nc2ccc(C(=O)N[C@H]3CCC[C@@H](Nc4ncc(Cl)c(-c5c[nH]c6ccccc56)n4)C3)cc2)CC1] | Compound 28 |
| 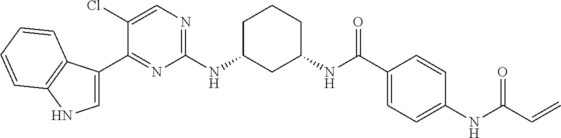 [SMILES: C=CC(=O)Nc1ccc(C(=O)N[C@H]2CCC[C@@H](Nc3ncc(Cl)c(-c4c[nH]c5ccccc45)n3)C2)cc1] | Compound 29 |
| 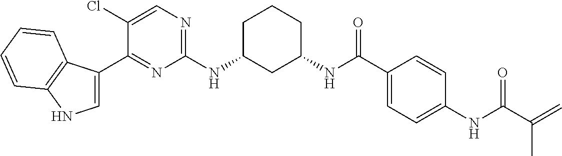 [SMILES: C=C(C)C(=O)Nc1ccc(C(=O)N[C@H]2CCC[C@@H](Nc3ncc(Cl)c(-c4c[nH]c5ccccc45)n3)C2)cc1] | Compound 30 |
| 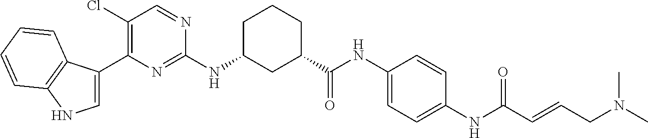 [SMILES: CN(C)C/C=C/C(=O)Nc1ccc(NC(=O)[C@H]2CCC[C@@H](Nc3ncc(Cl)c(-c4c[nH]c5ccccc45)n3)C2)cc1] | Compound 31 |
| 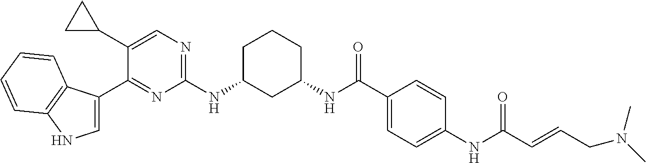 [SMILES: CN(C)C/C=C/C(=O)Nc1ccc(C(=O)N[C@H]2CCC[C@@H](Nc3ncc(C4CC4)c(-c4c[nH]c5ccccc45)n3)C2)cc1] | Compound 32 |
| 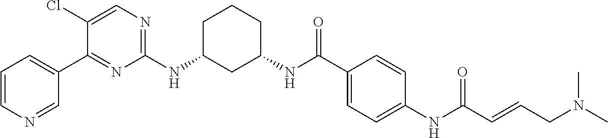 [SMILES: CN(C)C/C=C/C(=O)Nc1ccc(C(=O)N[C@H]2CCC[C@@H](Nc3ncc(Cl)c(-c4cccnc4)n3)C2)cc1] | Compound 33 |
| 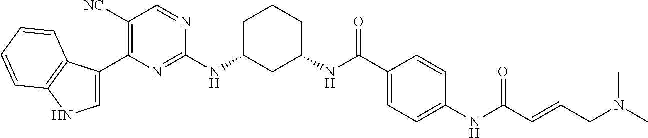 [SMILES: CN(C)C/C=C/C(=O)Nc1ccc(C(=O)N[C@H]2CCC[C@@H](Nc3ncc(C#N)c(-c4c[nH]c5ccccc45)n3)C2)cc1] | Compound 34 |
| 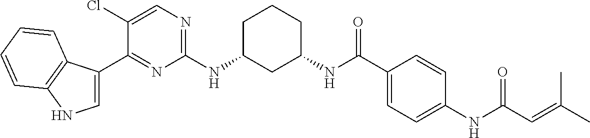 [SMILES: CC(C)=CC(=O)Nc1ccc(C(=O)N[C@H]2CCC[C@@H](Nc3ncc(Cl)c(-c4c[nH]c5ccccc45)n3)C2)cc1] | Compound 35 |
| 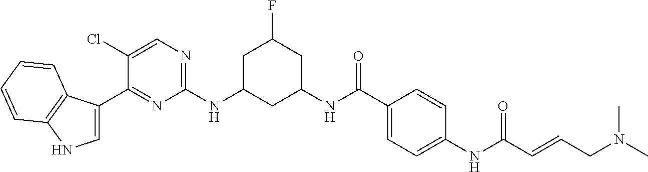 [SMILES: CN(C)C/C=C/C(=O)Nc1ccc(C(=O)NC2CC(F)CC(Nc3ncc(Cl)c(-c4c[nH]c5ccccc45)n3)C2)cc1] | Compound 36 |
| 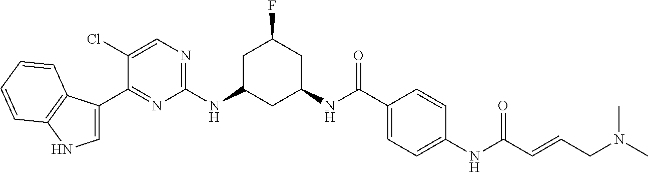 | Compound 37 |
| 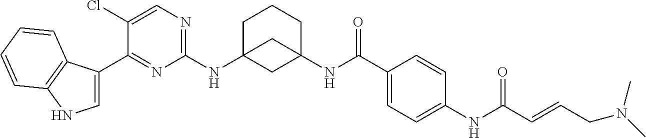 [SMILES: CN(C)C/C=C/C(=O)Nc1ccc(C(=O)NC23CCCC(Nc4ncc(Cl)c(-c5c[nH]c6ccccc56)n4)(C2)C3)cc1] | Compound 38 |
| 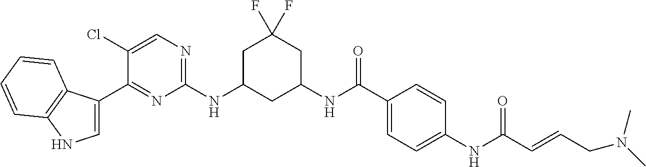 [SMILES: CN(C)C/C=C/C(=O)Nc1ccc(C(=O)NC2CC(Nc3ncc(Cl)c(-c4c[nH]c5ccccc45)n3)CC(F)(F)C2)cc1] | Compound 39 |
| 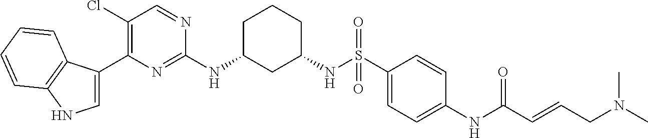 [SMILES: CN(C)C/C=C/C(=O)Nc1ccc(S(=O)(=O)N[C@H]2CCC[C@@H](Nc3ncc(Cl)c(-c4c[nH]c5ccccc45)n3)C2)cc1] | Compound 40 |
| 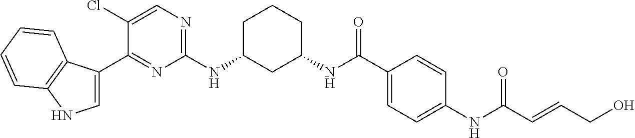 [SMILES: O=C(/C=C/CO)Nc1ccc(C(=O)N[C@H]2CCC[C@@H](Nc3ncc(Cl)c(-c4c[nH]c5ccccc45)n3)C2)cc1] | Compound 41 |
| 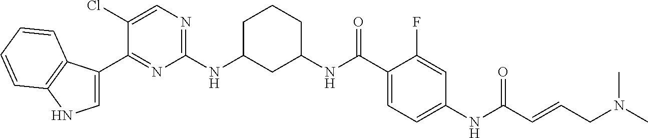 [SMILES: CN(C)C/C=C/C(=O)Nc1ccc(C(=O)NC2CCCC(Nc3ncc(Cl)c(-c4c[nH]c5ccccc45)n3)C2)c(F)c1] | Compound 42 |
| 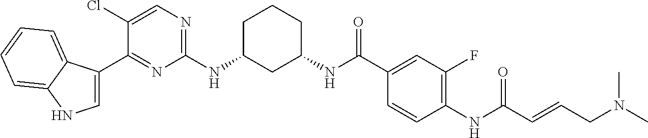 [SMILES: CN(C)C/C=C/C(=O)Nc1ccc(C(=O)N[C@H]2CCC[C@@H](Nc3ncc(Cl)c(-c4c[nH]c5ccccc45)n3)C2)cc1F] | Compound 43 |
| 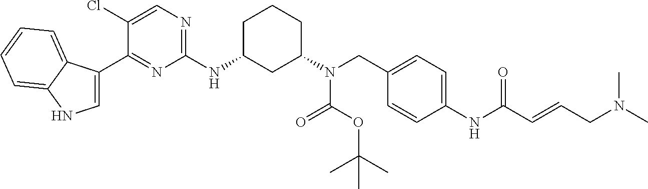 [SMILES: CN(C)C/C=C/C(=O)Nc1ccc(CN(C(=O)OC(C)(C)C)[C@H]2CCC[C@@H](Nc3ncc(Cl)c(-c4c[nH]c5ccccc45)n3)C2)cc1] | Compound 44 |
| 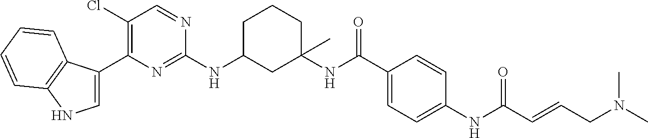 [SMILES: CN(C)C/C=C/C(=O)Nc1ccc(C(=O)NC2(C)CCCC(Nc3ncc(Cl)c(-c4c[nH]c5ccccc45)n3)C2)cc1] | Compound 45 |
| 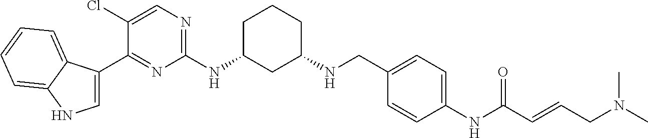 [SMILES: CN(C)C/C=C/C(=O)Nc1ccc(CN[C@H]2CCC[C@@H](Nc3ncc(Cl)c(-c4c[nH]c5ccccc45)n3)C2)cc1] | Compound 46 |
| 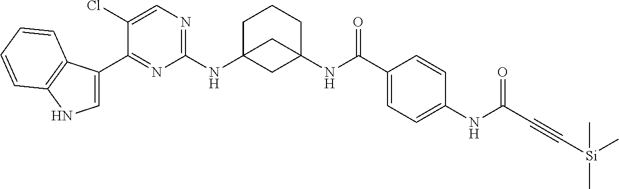 [SMILES: C[Si](C)(C)C#CC(=O)Nc1ccc(C(=O)NC23CCCC(Nc4ncc(Cl)c(-c5c[nH]c6ccccc56)n4)(C2)C3)cc1] | Compound 47 |
| 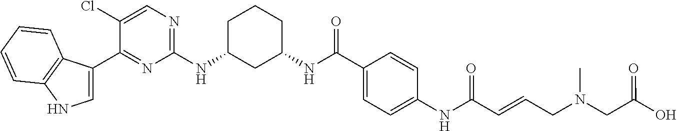 [SMILES: CN(C/C=C/C(=O)Nc1ccc(C(=O)N[C@H]2CCC[C@@H](Nc3ncc(Cl)c(-c4c[nH]c5ccccc45)n3)C2)cc1)CC(=O)O] | Compound 48 |
| 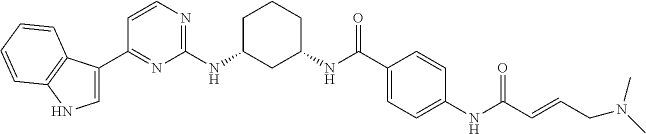 [SMILES: CN(C)C/C=C/C(=O)Nc1ccc(C(=O)N[C@H]2CCC[C@@H](Nc3nccc(-c4c[nH]c5ccccc45)n3)C2)cc1] | Compound 49 |
| 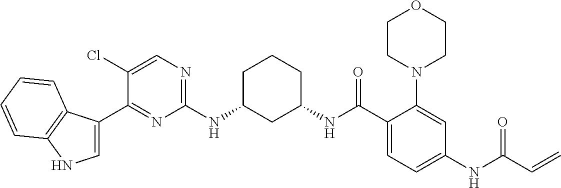 [SMILES: C=CC(=O)Nc1ccc(C(=O)N[C@H]2CCC[C@@H](Nc3ncc(Cl)c(-c4c[nH]c5ccccc45)n3)C2)c(N2CCOCC2)c1] | Compound 50 |
| 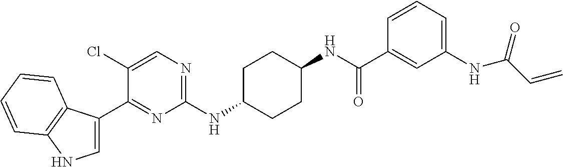 [SMILES: C=CC(=O)Nc1cccc(C(=O)N[C@H]2CC[C@H](Nc3ncc(Cl)c(-c4c[nH]c5ccccc45)n3)CC2)c1] | Compound 51 |
| 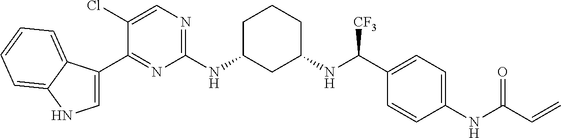 [SMILES: C=CC(=O)Nc1ccc([C@@H](N[C@H]2CCC[C@@H](Nc3ncc(Cl)c(-c4c[nH]c5ccccc45)n3)C2)C(F)(F)F)cc1] | Compound 52 |
| 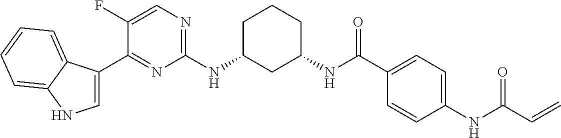 [SMILES: C=CC(=O)Nc1ccc(C(=O)N[C@H]2CCC[C@@H](Nc3ncc(F)c(-c4c[nH]c5ccccc45)n3)C2)cc1] | Compound 53 |
| 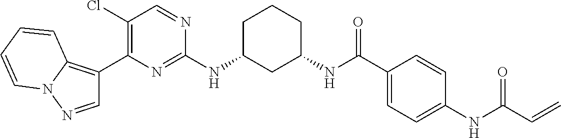 [SMILES: C=CC(=O)Nc1ccc(C(=O)N[C@H]2CCC[C@@H](Nc3ncc(Cl)c(-c4cnn5ccccc45)n3)C2)cc1] | Compound 54 |
| 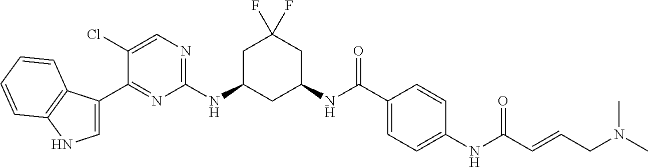 [SMILES: CN(C)C/C=C/C(=O)Nc1ccc(C(=O)N[C@H]2C[C@@H](Nc3ncc(Cl)c(-c4c[nH]c5ccccc45)n3)CC(F)(F)C2)cc1] | Compound 55 |
| 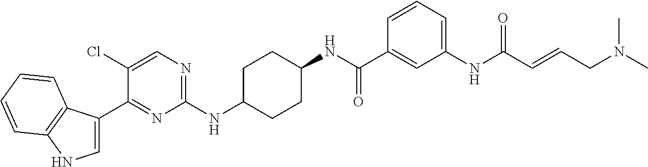 | Compound 56 |
| 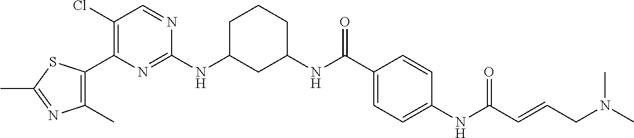 | Compound 57 |
| 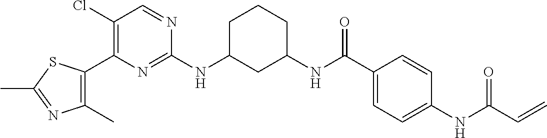 [SMILES: C=CC(=O)Nc1ccc(C(=O)NC2CCCC(Nc3ncc(Cl)c(-c4sc(C)nc4C)n3)C2)cc1] | Compound 58 |
| 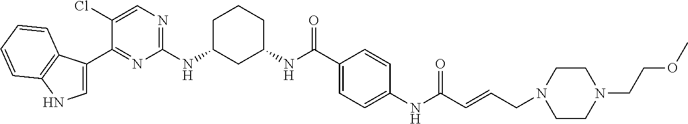 [SMILES: COCCN1CCN(C/C=C/C(=O)Nc2ccc(C(=O)N[C@H]3CCC[C@@H](Nc4ncc(Cl)c(-c5c[nH]c6ccccc56)n4)C3)cc2)CC1] | Compound 59 |
| 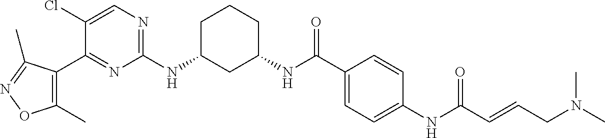 [SMILES: Cc1noc(C)c1-c1nc(N[C@@H]2CCC[C@H](NC(=O)c3ccc(NC(=O)/C=C/CN(C)C)cc3)C2)ncc1Cl] | Compound 60 |
| 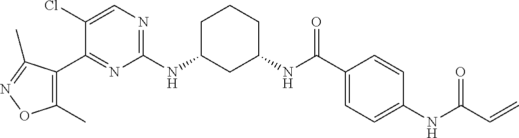 [SMILES: C=CC(=O)Nc1ccc(C(=O)N[C@H]2CCC[C@@H](Nc3ncc(Cl)c(-c4c(C)noc4C)n3)C2)cc1] | Compound 61 |
| 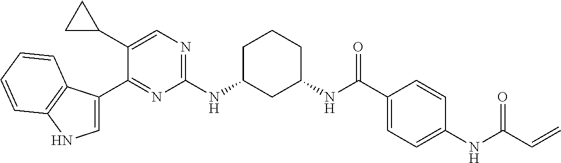 [SMILES: C=CC(=O)Nc1ccc(C(=O)N[C@H]2CCC[C@@H](Nc3ncc(C4CC4)c(-c4c[nH]c5ccccc45)n3)C2)cc1] | Compound 62 |
| 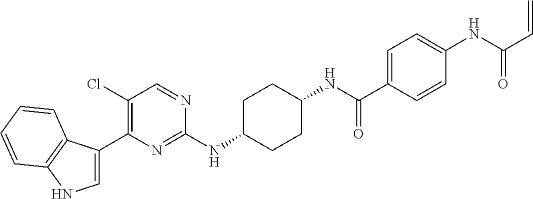 [SMILES: C=CC(=O)Nc1ccc(C(=O)N[C@H]2CC[C@@H](Nc3ncc(Cl)c(-c4c[nH]c5ccccc45)n3)CC2)cc1] | Compound 63 |
| 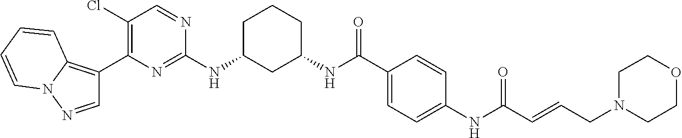 [SMILES: O=C(/C=C/CN1CCOCC1)Nc1ccc(C(=O)N[C@H]2CCC[C@@H](Nc3ncc(Cl)c(-c4cnn5ccccc45)n3)C2)cc1] | Compound 64 |
| 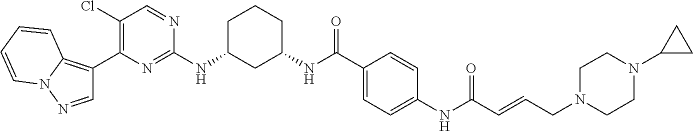 [SMILES: O=C(/C=C/CN1CCN(C2CC2)CC1)Nc1ccc(C(=O)N[C@H]2CCC[C@@H](Nc3ncc(Cl)c(-c4cnn5ccccc45)n3)C2)cc1] | Compound 65 |
| 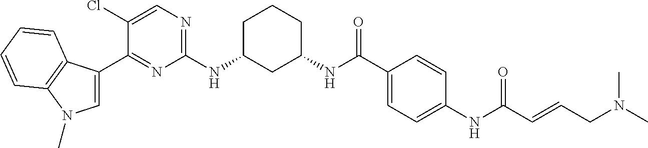 [SMILES: CN(C)C/C=C/C(=O)Nc1ccc(C(=O)N[C@H]2CCC[C@@H](Nc3ncc(Cl)c(-c4cn(C)c5ccccc45)n3)C2)cc1] | Compound 66 |
| 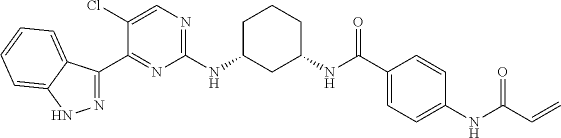 [SMILES: C=CC(=O)Nc1ccc(C(=O)N[C@H]2CCC[C@@H](Nc3ncc(Cl)c(-c4n[nH]c5ccccc45)n3)C2)cc1] | Compound 67 |
| 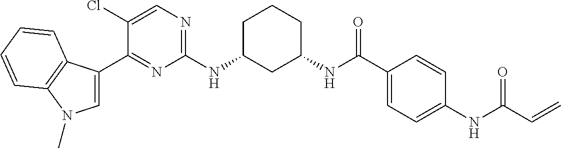 [SMILES: C=CC(=O)Nc1ccc(C(=O)N[C@H]2CCC[C@@H](Nc3ncc(Cl)c(-c4cn(C)c5ccccc45)n3)C2)cc1] | Compound 68 |
| 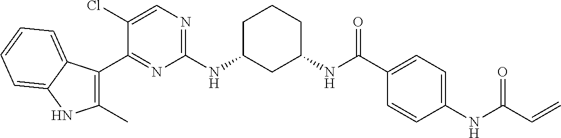 [SMILES: C=CC(=O)Nc1ccc(C(=O)N[C@H]2CCC[C@@H](Nc3ncc(Cl)c(-c4c(C)[nH]c5ccccc45)n3)C2)cc1] | Compound 69 |
| 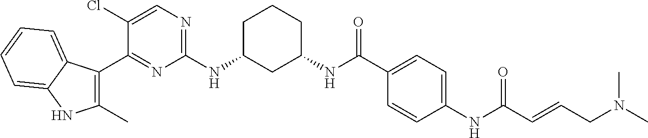 [SMILES: Cc1[nH]c2ccccc2c1-c1nc(N[C@@H]2CCC[C@H](NC(=O)c3ccc(NC(=O)/C=C/CN(C)C)cc3)C2)ncc1Cl] | Compound 70 |
| 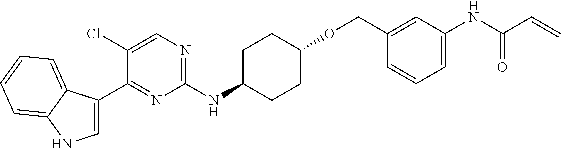 [SMILES: C=CC(=O)Nc1cccc(CO[C@H]2CC[C@H](Nc3ncc(Cl)c(-c4c[nH]c5ccccc45)n3)CC2)c1] | Compound 71 |
| 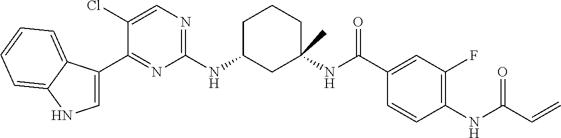 [SMILES: C=CC(=O)Nc1ccc(C(=O)N[C@@]2(C)CCC[C@@H](Nc3ncc(Cl)c(-c4c[nH]c5ccccc45)n3)C2)cc1F] | Compound 72 |
| 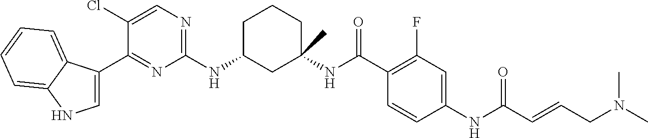 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)c(F)c1] | Compound 73 |
| 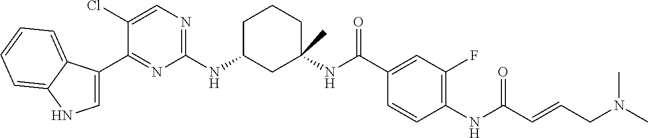 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)cc1F] | Compound 74 |
| 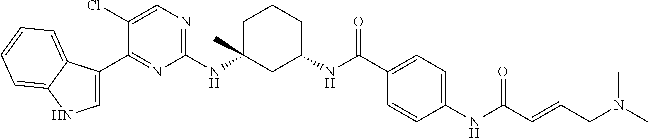 [SMILES: CN(C)C/C=C/C(=O)Nc1ccc(C(=O)N[C@H]2CCC[C@@](C)(Nc3ncc(Cl)c(-c4c[nH]c5ccccc45)n3)C2)cc1] | Compound 75 |
| 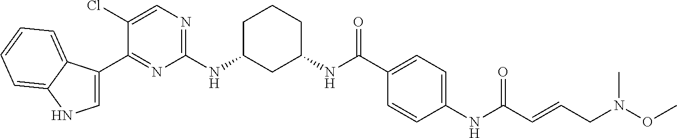 [SMILES: CON(C)C/C=C/C(=O)Nc1ccc(C(=O)N[C@H]2CCC[C@@H](Nc3ncc(Cl)c(-c4c[nH]c5ccccc45)n3)C2)cc1] | Compound 76 |
| 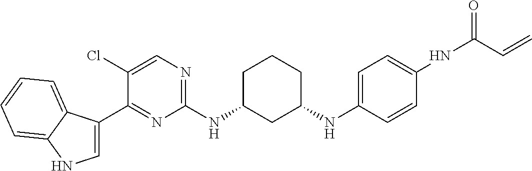 [SMILES: C=CC(=O)Nc1ccc(N[C@H]2CCC[C@@H](Nc3ncc(Cl)c(-c4c[nH]c5ccccc45)n3)C2)cc1] | Compound 77 |
| 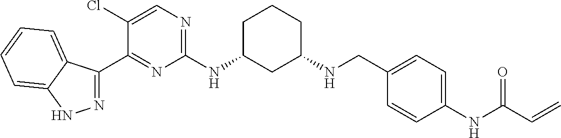 [SMILES: C=CC(=O)Nc1ccc(CN[C@H]2CCC[C@@H](Nc3ncc(Cl)c(-c4n[nH]c5ccccc45)n3)C2)cc1] | Compound 78 |
| 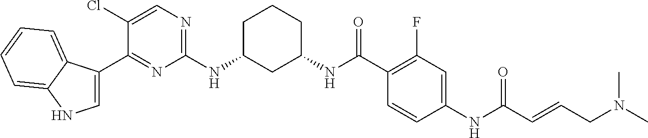 [SMILES: CN(C)C/C=C/C(=O)Nc1ccc(C(=O)N[C@H]2CCC[C@@H](Nc3ncc(Cl)c(-c4c[nH]c5ccccc45)n3)C2)c(F)c1] | Compound 79 |
| 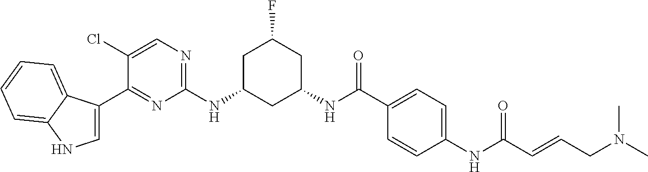 [SMILES: CN(C)C/C=C/C(=O)Nc1ccc(C(=O)N[C@H]2C[C@@H](F)C[C@@H](Nc3ncc(Cl)c(-c4c[nH]c5ccccc45)n3)C2)cc1] | Compound 80 |
| 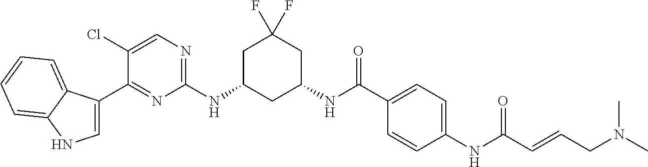 [SMILES: CN(C)C/C=C/C(=O)Nc1ccc(C(=O)N[C@@H]2C[C@H](Nc3ncc(Cl)c(-c4c[nH]c5ccccc45)n3)CC(F)(F)C2)cc1] | Compound 81 |
| 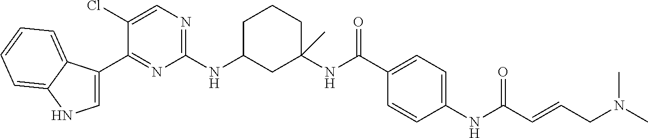 [SMILES: CN(C)C/C=C/C(=O)Nc1ccc(C(=O)NC2(C)CCCC(Nc3ncc(Cl)c(-c4c[nH]c5ccccc45)n3)C2)cc1] | Compound 82 |
| 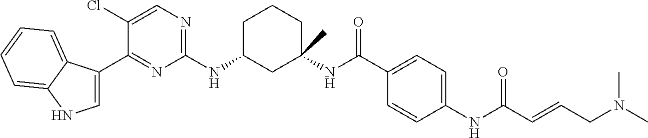 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)cc1] | Compound 83 |
| 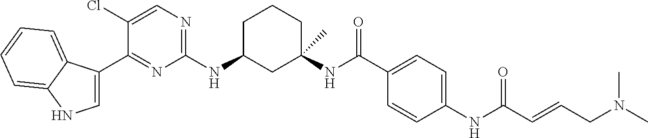 [SMILES: CN(C)C/C=C/C(=O)Nc1ccc(C(=O)N[C@]2(C)CCC[C@H](Nc3ncc(Cl)c(-c4c[nH]c5ccccc45)n3)C2)cc1] | Compound 84 |
| 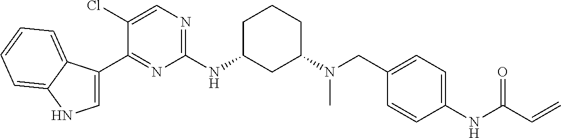 [SMILES: C=CC(=O)Nc1ccc(CN(C)[C@H]2CCC[C@@H](Nc3ncc(Cl)c(-c4c[nH]c5ccccc45)n3)C2)cc1] | Compound 85 |
| 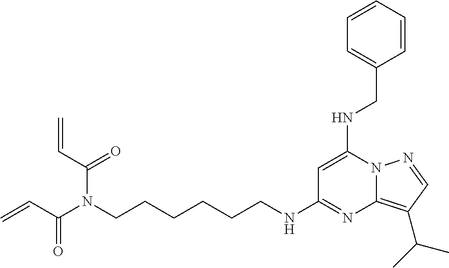 [SMILES: C=CC(=O)N(CCCCCCNc1cc(NCc2ccccc2)n2ncc(C(C)C)c2n1)C(=O)C=C] | Compound 86 |
| 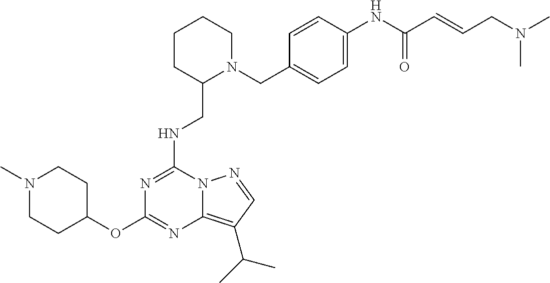 | Compound 87 |
| 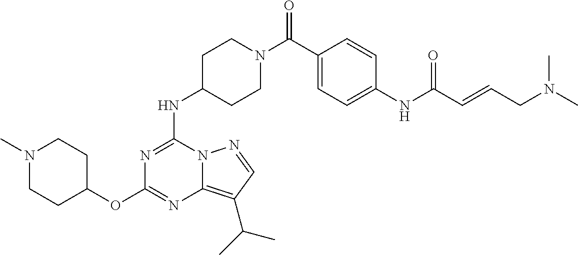 | Compound 88 |
| 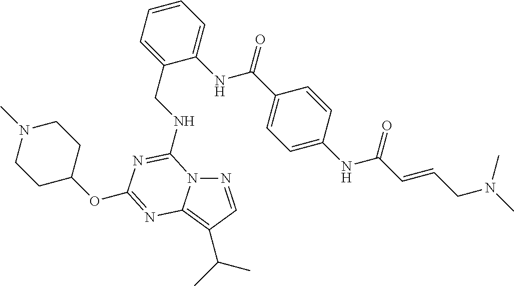 | Compound 89 |
| 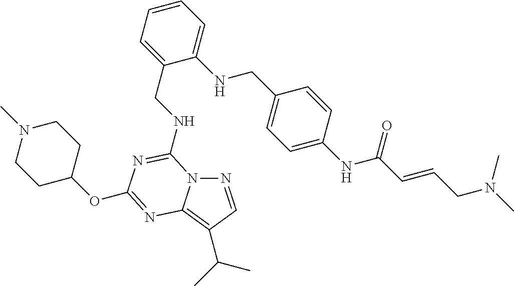 | Compound 90 |
| 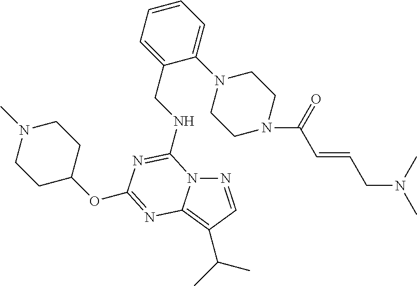 | Compound 91 |
| 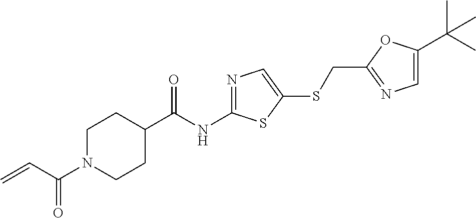 [SMILES: C=CC(=O)N1CCC(C(=O)Nc2ncc(SCc3ncc(C(C)(C)C)o3)s2)CC1] | Compound 92 |
| 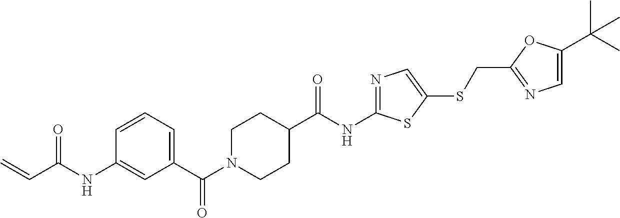 [SMILES: C=CC(=O)Nc1cccc(C(=O)N2CCC(C(=O)Nc3ncc(SCc4ncc(C(C)(C)C)o4)s3)CC2)c1] | Compound 93 |
| 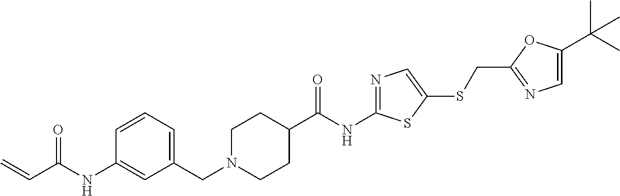 [SMILES: C=CC(=O)Nc1cccc(CN2CCC(C(=O)Nc3ncc(SCc4ncc(C(C)(C)C)o4)s3)CC2)c1] | Compound 94 |
| 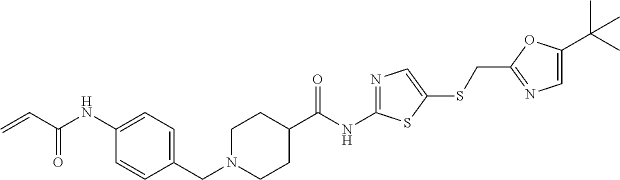 [SMILES: C=CC(=O)Nc1ccc(CN2CCC(C(=O)Nc3ncc(SCc4ncc(C(C)(C)C)o4)s3)CC2)cc1] | Compound 95 |
| 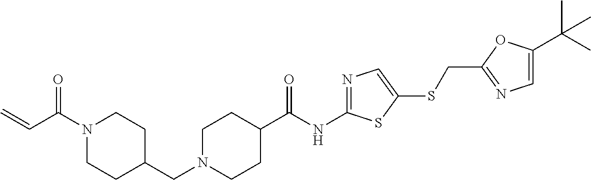 [SMILES: C=CC(=O)N1CCC(CN2CCC(C(=O)Nc3ncc(SCc4ncc(C(C)(C)C)o4)s3)CC2)CC1] | Compound 96 |
| 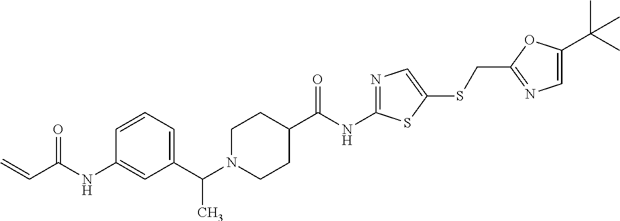 [SMILES: C=CC(=O)Nc1cccc(C(C)N2CCC(C(=O)Nc3ncc(SCc4ncc(C(C)(C)C)o4)s3)CC2)c1] | Compound 97 |
| 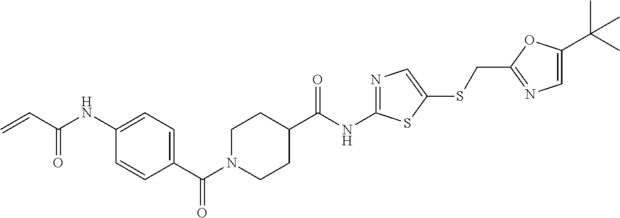 [SMILES: C=CC(=O)Nc1ccc(C(=O)N2CCC(C(=O)Nc3ncc(SCc4ncc(C(C)(C)C)o4)s3)CC2)cc1] | Compound 98 |
| 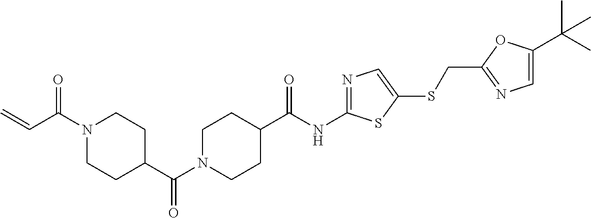 [SMILES: C=CC(=O)N1CCC(C(=O)N2CCC(C(=O)Nc3ncc(SCc4ncc(C(C)(C)C)o4)s3)CC2)CC1] | Compound 99 |
| 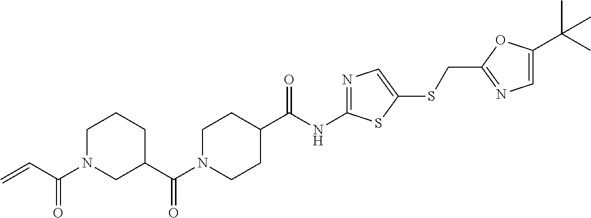 [SMILES: C=CC(=O)N1CCCC(C(=O)N2CCC(C(=O)Nc3ncc(SCc4ncc(C(C)(C)C)o4)s3)CC2)C1] | Compound 100 |
| 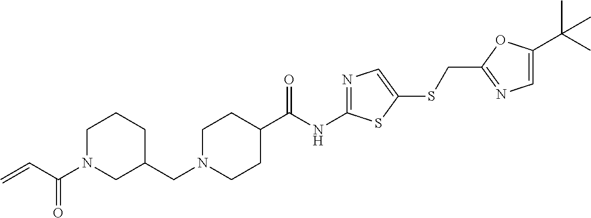 [SMILES: C=CC(=O)N1CCCC(CN2CCC(C(=O)Nc3ncc(SCc4ncc(C(C)(C)C)o4)s3)CC2)C1] | Compound 101 |
| 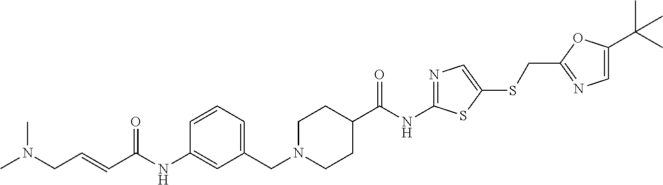 [SMILES: CN(C)C/C=C/C(=O)Nc1cccc(CN2CCC(C(=O)Nc3ncc(SCc4ncc(C(C)(C)C)o4)s3)CC2)c1] | Compound 102 |
| 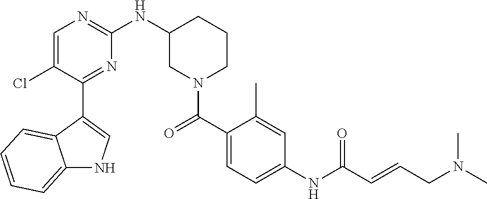 [SMILES: Cc1cc(NC(=O)/C=C/CN(C)C)ccc1C(=O)N1CCCC(Nc2ncc(Cl)c(-c3c[nH]c4ccccc34)n2)C1] | Compound 103 |
| 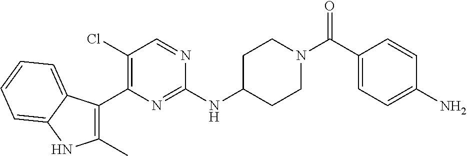 [SMILES: Cc1[nH]c2ccccc2c1-c1nc(NC2CCN(C(=O)c3ccc(N)cc3)CC2)ncc1Cl] | Compound 104 |
| 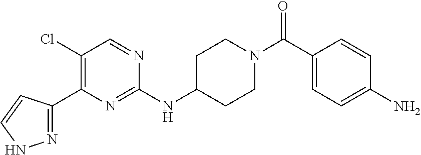 [SMILES: Nc1ccc(C(=O)N2CCC(Nc3ncc(Cl)c(-c4cc[nH]n4)n3)CC2)cc1] | Compound 105 |
| 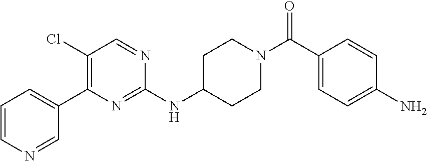 [SMILES: Nc1ccc(C(=O)N2CCC(Nc3ncc(Cl)c(-c4cccnc4)n3)CC2)cc1] | Compound 106 |
| 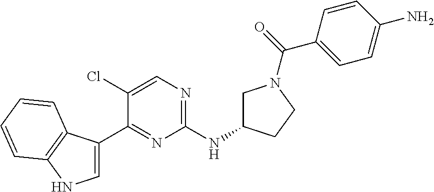 [SMILES: Nc1ccc(C(=O)N2CC[C@H](Nc3ncc(Cl)c(-c4c[nH]c5ccccc45)n3)C2)cc1] | Compound 107 |
| 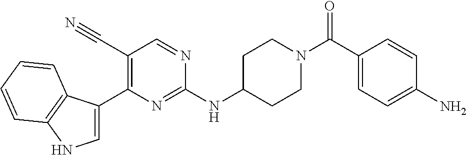 [SMILES: N#Cc1cnc(NC2CCN(C(=O)c3ccc(N)cc3)CC2)nc1-c1c[nH]c2ccccc12] | Compound 108 |
| 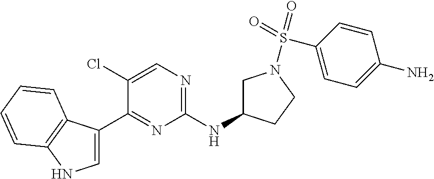 [SMILES: Nc1ccc(S(=O)(=O)N2CC[C@@H](Nc3ncc(Cl)c(-c4c[nH]c5ccccc45)n3)C2)cc1] | Compound 109 |
| 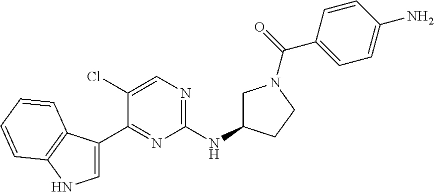 [SMILES: Nc1ccc(C(=O)N2CC[C@@H](Nc3ncc(Cl)c(-c4c[nH]c5ccccc45)n3)C2)cc1] | Compound 110 |
| 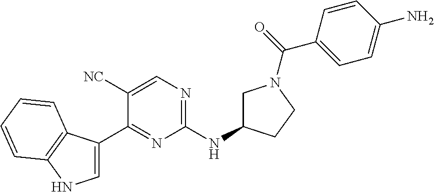 [SMILES: N#Cc1cnc(N[C@@H]2CCN(C(=O)c3ccc(N)cc3)C2)nc1-c1c[nH]c2ccccc12] | Compound 111 |
| 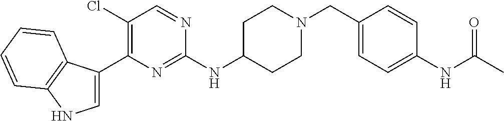 [SMILES: CC(=O)Nc1ccc(CN2CCC(Nc3ncc(Cl)c(-c4c[nH]c5ccccc45)n3)CC2)cc1] | Compound 112 |
| 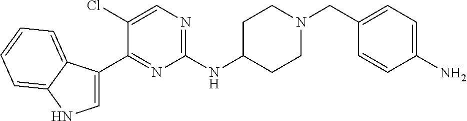 [SMILES: Nc1ccc(CN2CCC(Nc3ncc(Cl)c(-c4c[nH]c5ccccc45)n3)CC2)cc1] | Compound 113 |
| 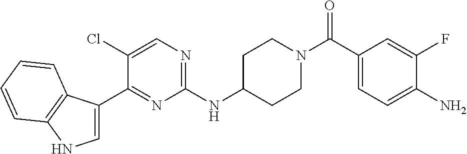 [SMILES: Nc1ccc(C(=O)N2CCC(Nc3ncc(Cl)c(-c4c[nH]c5ccccc45)n3)CC2)cc1F] | Compound 114 |
| 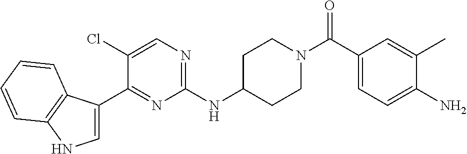 [SMILES: Cc1cc(C(=O)N2CCC(Nc3ncc(Cl)c(-c4c[nH]c5ccccc45)n3)CC2)ccc1N] | Compound 115 |
| 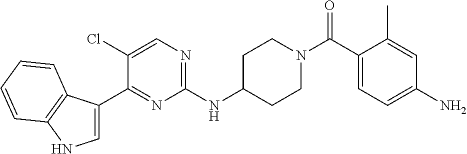 [SMILES: Cc1cc(N)ccc1C(=O)N1CCC(Nc2ncc(Cl)c(-c3c[nH]c4ccccc34)n2)CC1] | Compound 116 |
| 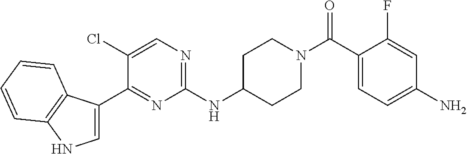 [SMILES: Nc1ccc(C(=O)N2CCC(Nc3ncc(Cl)c(-c4c[nH]c5ccccc45)n3)CC2)c(F)c1] | Compound 117 |
| 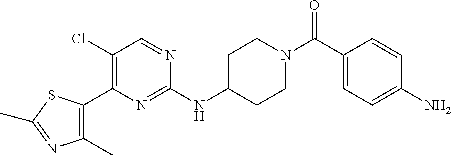 [SMILES: Cc1nc(C)c(-c2nc(NC3CCN(C(=O)c4ccc(N)cc4)CC3)ncc2Cl)s1] | Compound 118 |
| 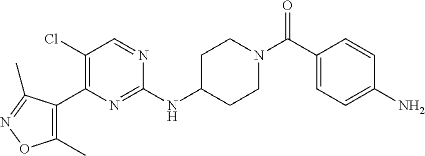 [SMILES: Cc1noc(C)c1-c1nc(NC2CCN(C(=O)c3ccc(N)cc3)CC2)ncc1Cl] | Compound 119 |
| 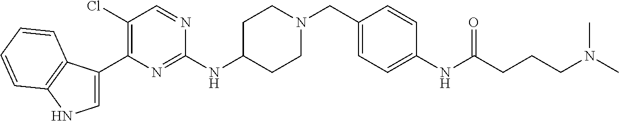 [SMILES: CN(C)CCCC(=O)Nc1ccc(CN2CCC(Nc3ncc(Cl)c(-c4c[nH]c5ccccc45)n3)CC2)cc1] | Compound 120 |
| 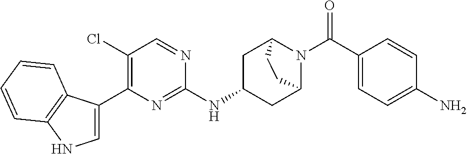 [SMILES: Nc1ccc(C(=O)N2[C@@H]3CC[C@H]2C[C@@H](Nc2ncc(Cl)c(-c4c[nH]c5ccccc45)n2)C3)cc1] | Compound 121 |
| 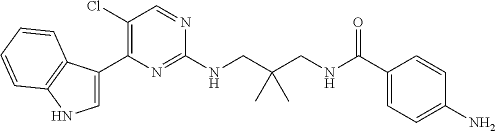 [SMILES: CC(C)(CNC(=O)c1ccc(N)cc1)CNc1ncc(Cl)c(-c2c[nH]c3ccccc23)n1] | Compound 122 |
| 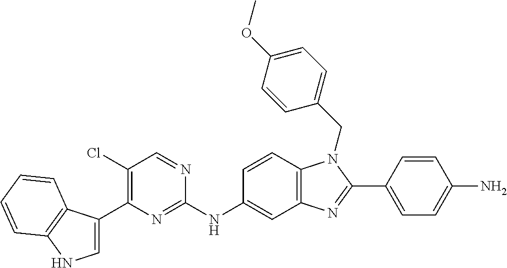 [SMILES: COc1ccc(Cn2c(-c3ccc(N)cc3)nc3cc(Nc4ncc(Cl)c(-c5c[nH]c6ccccc56)n4)ccc32)cc1] | Compound 123 |
| 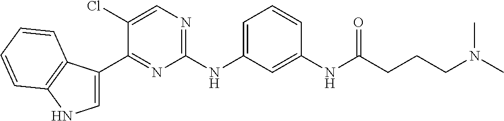 [SMILES: CN(C)CCCC(=O)Nc1cccc(Nc2ncc(Cl)c(-c3c[nH]c4ccccc34)n2)c1] | Compound 124 |
| 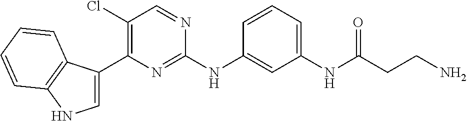 [SMILES: NCCC(=O)Nc1cccc(Nc2ncc(Cl)c(-c3c[nH]c4ccccc34)n2)c1] | Compound 125 |
| 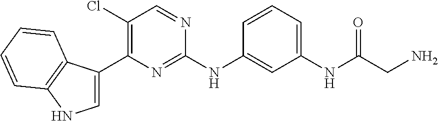 [SMILES: NCC(=O)Nc1cccc(Nc2ncc(Cl)c(-c3c[nH]c4ccccc34)n2)c1] | Compound 126 |
| 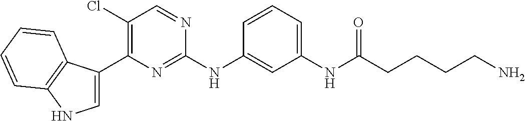 [SMILES: NCCCCC(=O)Nc1cccc(Nc2ncc(Cl)c(-c3c[nH]c4ccccc34)n2)c1] | Compound 127 |
| 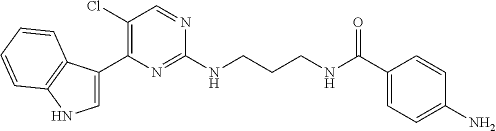 [SMILES: Nc1ccc(C(=O)NCCCNc2ncc(Cl)c(-c3c[nH]c4ccccc34)n2)cc1] | Compound 128 |
| 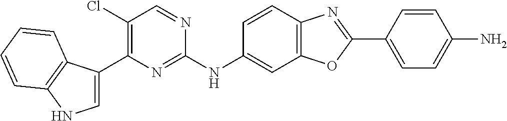 [SMILES: Nc1ccc(-c2nc3ccc(Nc4ncc(Cl)c(-c5c[nH]c6ccccc56)n4)cc3o2)cc1] | Compound 129 |
| 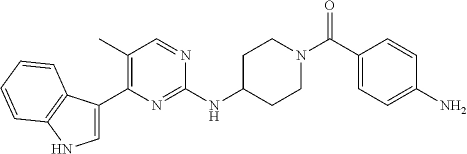 [SMILES: Cc1cnc(NC2CCN(C(=O)c3ccc(N)cc3)CC2)nc1-c1c[nH]c2ccccc12] | Compound 130 |
| 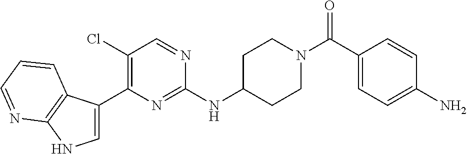 [SMILES: Nc1ccc(C(=O)N2CCC(Nc3ncc(Cl)c(-c4c[nH]c5ncccc45)n3)CC2)cc1] | Compound 131 |
| 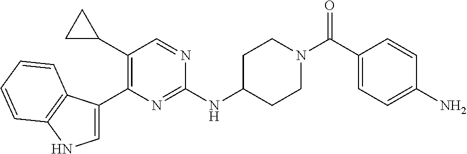 [SMILES: Nc1ccc(C(=O)N2CCC(Nc3ncc(C4CC4)c(-c4c[nH]c5ccccc45)n3)CC2)cc1] | Compound 132 |
| 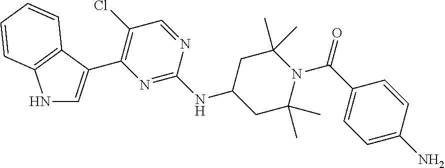 [SMILES: CC1(C)CC(Nc2ncc(Cl)c(-c3c[nH]c4ccccc34)n2)CC(C)(C)N1C(=O)c1ccc(N)cc1] | Compound 133 |
| 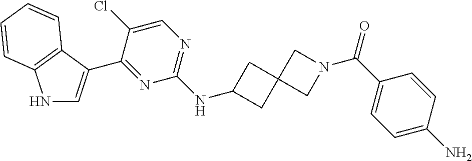 [SMILES: Nc1ccc(C(=O)N2CC3(CC(Nc4ncc(Cl)c(-c5c[nH]c6ccccc56)n4)C3)C2)cc1] | Compound 134 |
| 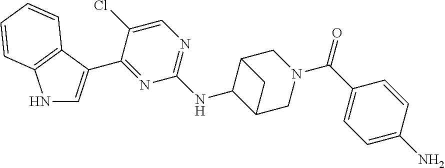 [SMILES: Nc1ccc(C(=O)N2CC3CC(C2)C3Nc2ncc(Cl)c(-c3c[nH]c4ccccc34)n2)cc1] | Compound 135 |
| 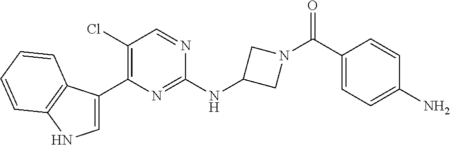 [SMILES: Nc1ccc(C(=O)N2CC(Nc3ncc(Cl)c(-c4c[nH]c5ccccc45)n3)C2)cc1] | Compound 136 |
| 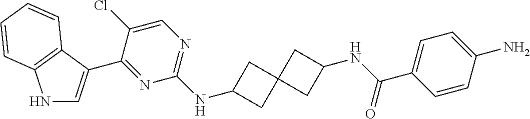 [SMILES: Nc1ccc(C(=O)NC2CC3(C2)CC(Nc2ncc(Cl)c(-c4c[nH]c5ccccc45)n2)C3)cc1] | Compound 137 |
| 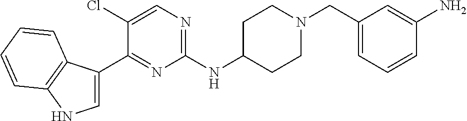 [SMILES: Nc1cccc(CN2CCC(Nc3ncc(Cl)c(-c4c[nH]c5ccccc45)n3)CC2)c1] | Compound 138 |
| 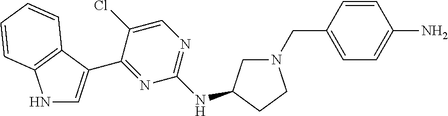 [SMILES: Nc1ccc(CN2CC[C@@H](Nc3ncc(Cl)c(-c4c[nH]c5ccccc45)n3)C2)cc1] | Compound 139 |
| 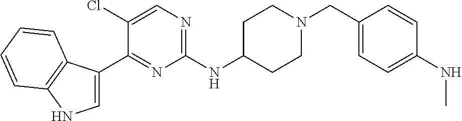 [SMILES: CNc1ccc(CN2CCC(Nc3ncc(Cl)c(-c4c[nH]c5ccccc45)n3)CC2)cc1] | Compound 140 |
| 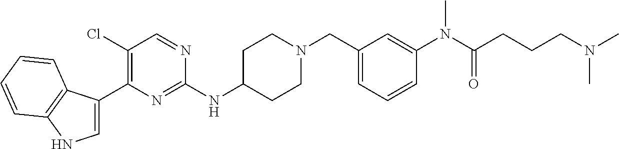 [SMILES: CN(C)CCCC(=O)N(C)c1cccc(CN2CCC(Nc3ncc(Cl)c(-c4c[nH]c5ccccc45)n3)CC2)c1] | Compound 141 |
| 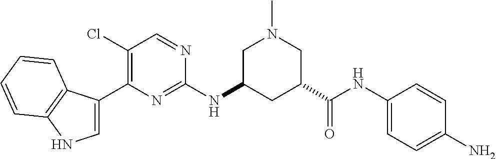 | Compound 142 |
| 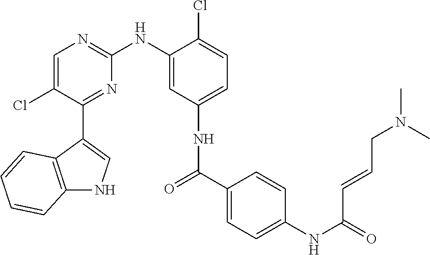 [SMILES: CN(C)C/C=C/C(=O)Nc1ccc(C(=O)Nc2ccc(Cl)c(Nc3ncc(Cl)c(-c4c[nH]c5ccccc45)n3)c2)cc1] | Compound 143 |
| 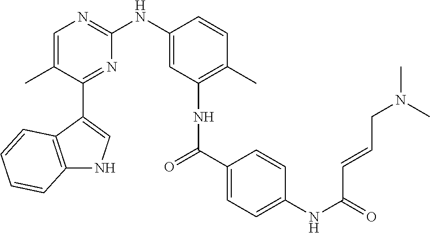 [SMILES: Cc1ccc(Nc2ncc(C)c(-c3c[nH]c4ccccc34)n2)cc1NC(=O)c1ccc(NC(=O)/C=C/CN(C)C)cc1] | Compound 144 |
| 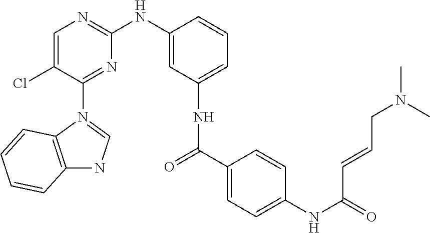 | Compound 145 |
| 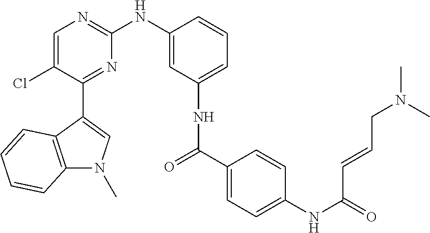 [SMILES: CN(C)C/C=C/C(=O)Nc1ccc(C(=O)Nc2cccc(Nc3ncc(Cl)c(-c4cn(C)c5ccccc45)n3)c2)cc1] | Compound 146 |
| 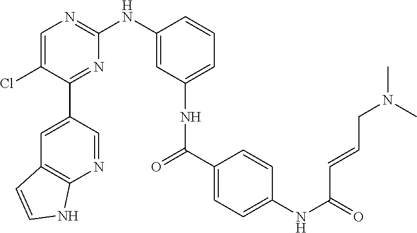 [SMILES: CN(C)C/C=C/C(=O)Nc1ccc(C(=O)Nc2cccc(Nc3ncc(Cl)c(-c4cnc5[nH]ccc5c4)n3)c2)cc1] | Compound 147 |
| 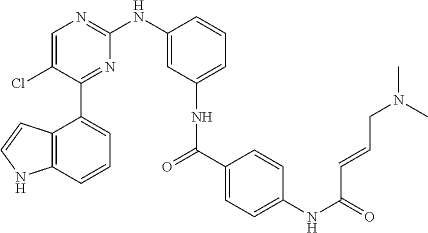 [SMILES: CN(C)C/C=C/C(=O)Nc1ccc(C(=O)Nc2cccc(Nc3ncc(Cl)c(-c4cccc5[nH]ccc45)n3)c2)cc1] | Compound 148 |
| 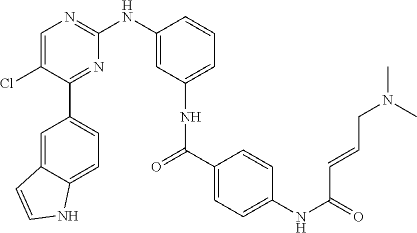 [SMILES: CN(C)C/C=C/C(=O)Nc1ccc(C(=O)Nc2cccc(Nc3ncc(Cl)c(-c4ccc5[nH]ccc5c4)n3)c2)cc1] | Compound 149 |
| 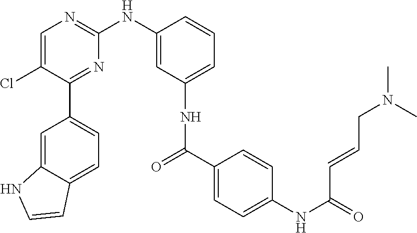 [SMILES: CN(C)C/C=C/C(=O)Nc1ccc(C(=O)Nc2cccc(Nc3ncc(Cl)c(-c4ccc5cc[nH]c5c4)n3)c2)cc1] | Compound 150 |
| 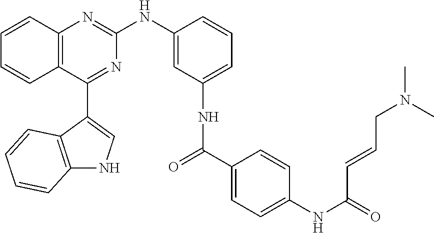 [SMILES: CN(C)C/C=C/C(=O)Nc1ccc(C(=O)Nc2cccc(Nc3nc(-c4c[nH]c5ccccc45)c4ccccc4n3)c2)cc1] | Compound 151 |
| 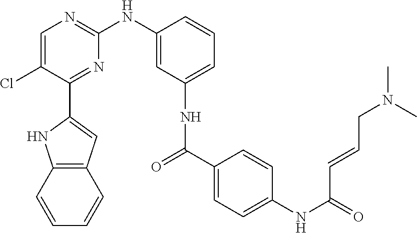 [SMILES: CN(C)C/C=C/C(=O)Nc1ccc(C(=O)Nc2cccc(Nc3ncc(Cl)c(-c4cc5ccccc5[nH]4)n3)c2)cc1] | Compound 152 |
| 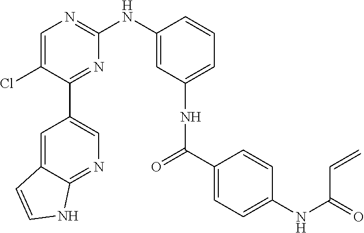 [SMILES: C=CC(=O)Nc1ccc(C(=O)Nc2cccc(Nc3ncc(Cl)c(-c4cnc5[nH]ccc5c4)n3)c2)cc1] | Compound 153 |
| 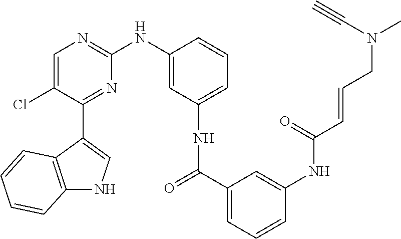 [SMILES: C#CN(C)C/C=C/C(=O)Nc1cccc(C(=O)Nc2cccc(Nc3ncc(Cl)c(-c4c[nH]c5ccccc45)n3)c2)c1] | Compound 154 |
| 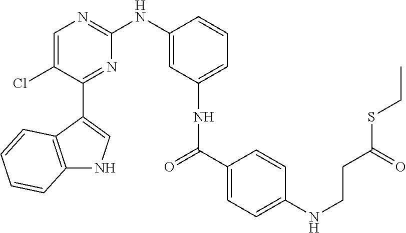 [SMILES: CCSC(=O)CCNc1ccc(C(=O)Nc2cccc(Nc3ncc(Cl)c(-c4c[nH]c5ccccc45)n3)c2)cc1] | Compound 155 |
| 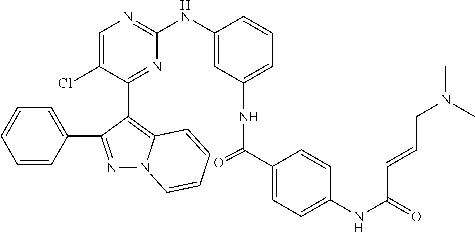 [SMILES: CN(C)C/C=C/C(=O)Nc1ccc(C(=O)Nc2cccc(Nc3ncc(Cl)c(-c4c(-c5ccccc5)nn5ccccc45)n3)c2)cc1] | Compound 156 |
| 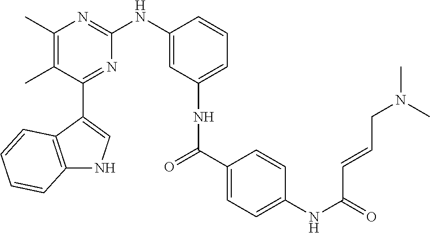 [SMILES: Cc1nc(Nc2cccc(NC(=O)c3ccc(NC(=O)/C=C/CN(C)C)cc3)c2)nc(-c2c[nH]c3ccccc23)c1C] | Compound 157 |
| 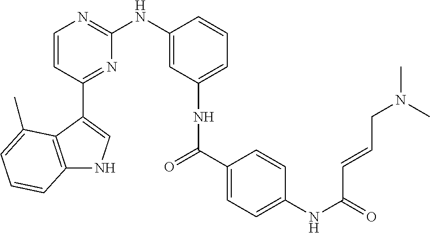 [SMILES: Cc1cccc2[nH]cc(-c3ccnc(Nc4cccc(NC(=O)c5ccc(NC(=O)/C=C/CN(C)C)cc5)c4)n3)c12] | Compound 158 |
| 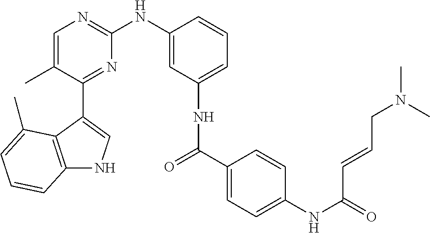 [SMILES: Cc1cnc(Nc2cccc(NC(=O)c3ccc(NC(=O)/C=C/CN(C)C)cc3)c2)nc1-c1c[nH]c2cccc(C)c12] | Compound 159 |
| 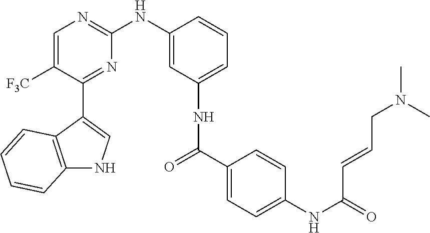 [SMILES: CN(C)C/C=C/C(=O)Nc1ccc(C(=O)Nc2cccc(Nc3ncc(C(F)(F)F)c(-c4c[nH]c5ccccc45)n3)c2)cc1] | Compound 160 |
| 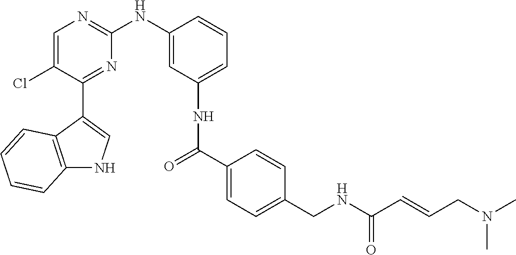 [SMILES: CN(C)C/C=C/C(=O)NCc1ccc(C(=O)Nc2cccc(Nc3ncc(Cl)c(-c4c[nH]c5ccccc45)n3)c2)cc1] | Compound 161 |
| 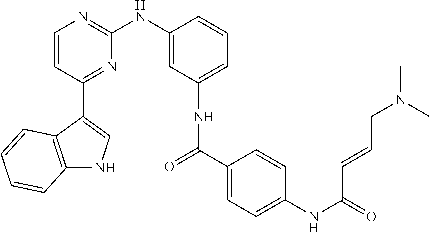 [SMILES: CN(C)C/C=C/C(=O)Nc1ccc(C(=O)Nc2cccc(Nc3nccc(-c4c[nH]c5ccccc45)n3)c2)cc1] | Compound 162 |
| 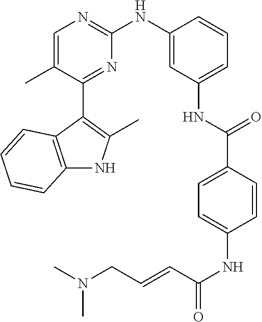 [SMILES: Cc1cnc(Nc2cccc(NC(=O)c3ccc(NC(=O)/C=C/CN(C)C)cc3)c2)nc1-c1c(C)[nH]c2ccccc12] | Compound 163 |
| 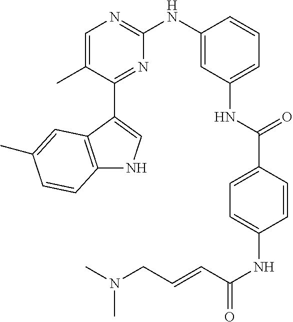 [SMILES: Cc1ccc2[nH]cc(-c3nc(Nc4cccc(NC(=O)c5ccc(NC(=O)/C=C/CN(C)C)cc5)c4)ncc3C)c2c1] | Compound 164 |
| 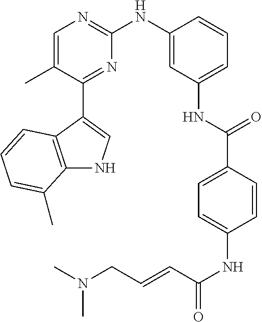 [SMILES: Cc1cnc(Nc2cccc(NC(=O)c3ccc(NC(=O)/C=C/CN(C)C)cc3)c2)nc1-c1c[nH]c2c(C)cccc12] | Compound 165 |
| 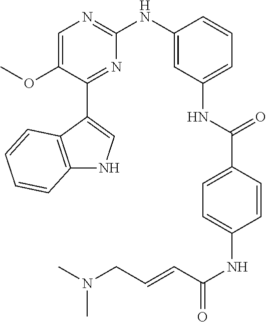 [SMILES: COc1cnc(Nc2cccc(NC(=O)c3ccc(NC(=O)/C=C/CN(C)C)cc3)c2)nc1-c1c[nH]c2ccccc12] | Compound 166 |
| 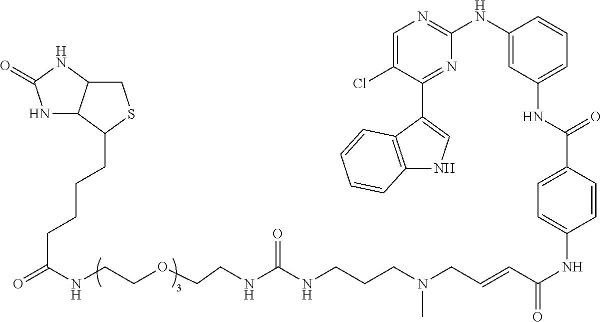 | Compound 167 |
| 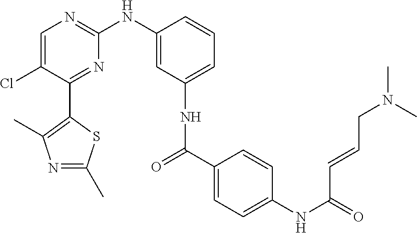 [SMILES: Cc1nc(C)c(-c2nc(Nc3cccc(NC(=O)c4ccc(NC(=O)/C=C/CN(C)C)cc4)c3)ncc2Cl)s1] | Compound 168 |
| 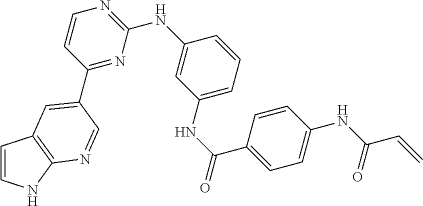 [SMILES: C=CC(=O)Nc1ccc(C(=O)Nc2cccc(Nc3nccc(-c4cnc5[nH]ccc5c4)n3)c2)cc1] | Compound 169 |
| 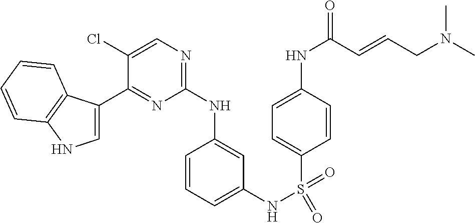 [SMILES: CN(C)C/C=C/C(=O)Nc1ccc(S(=O)(=O)Nc2cccc(Nc3ncc(Cl)c(-c4c[nH]c5ccccc45)n3)c2)cc1] | Compound 170 |
| 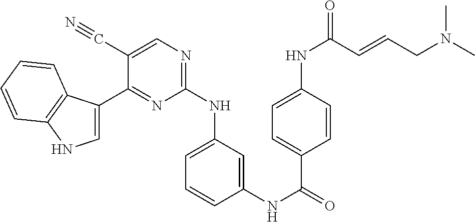 [SMILES: CN(C)C/C=C/C(=O)Nc1ccc(C(=O)Nc2cccc(Nc3ncc(C#N)c(-c4c[nH]c5ccccc45)n3)c2)cc1] | Compound 171 |
| 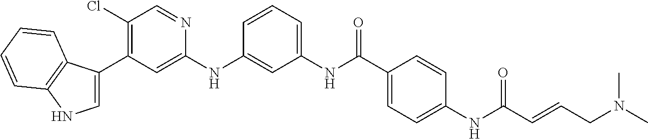 [SMILES: CN(C)C/C=C/C(=O)Nc1ccc(C(=O)Nc2cccc(Nc3cc(-c4c[nH]c5ccccc45)c(Cl)cn3)c2)cc1] | Compound 172 |
| 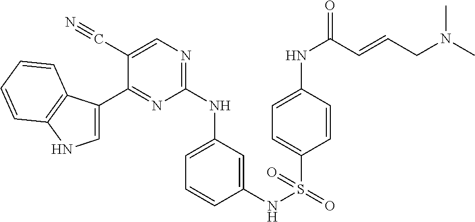 [SMILES: CN(C)C/C=C/C(=O)Nc1ccc(S(=O)(=O)Nc2cccc(Nc3ncc(C#N)c(-c4c[nH]c5ccccc45)n3)c2)cc1] | Compound 173 |
| 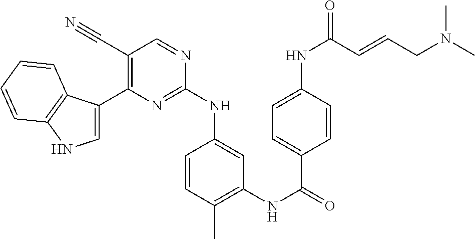 [SMILES: Cc1ccc(Nc2ncc(C#N)c(-c3c[nH]c4ccccc34)n2)cc1NC(=O)c1ccc(NC(=O)/C=C/CN(C)C)cc1] | Compound 174 |
| 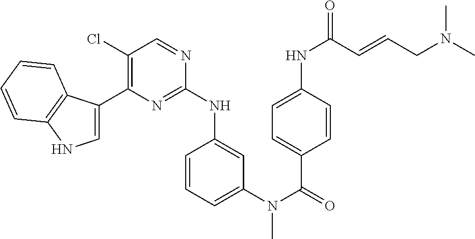 [SMILES: CN(C)C/C=C/C(=O)Nc1ccc(C(=O)N(C)c2cccc(Nc3ncc(Cl)c(-c4c[nH]c5ccccc45)n3)c2)cc1] | Compound 175 |
| 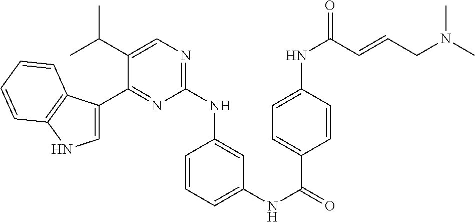 [SMILES: CC(C)c1cnc(Nc2cccc(NC(=O)c3ccc(NC(=O)/C=C/CN(C)C)cc3)c2)nc1-c1c[nH]c2ccccc12] | Compound 176 |
| 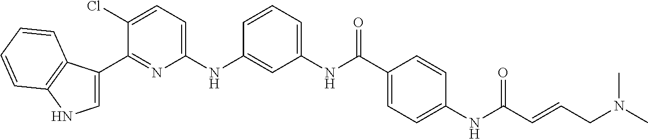 [SMILES: CN(C)C/C=C/C(=O)Nc1ccc(C(=O)Nc2cccc(Nc3ccc(Cl)c(-c4c[nH]c5ccccc45)n3)c2)cc1] | Compound 177 |
| 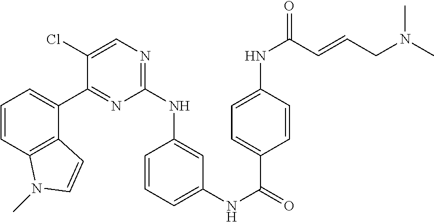 [SMILES: CN(C)C/C=C/C(=O)Nc1ccc(C(=O)Nc2cccc(Nc3ncc(Cl)c(-c4cccc5c4ccn5C)n3)c2)cc1] | Compound 178 |
| 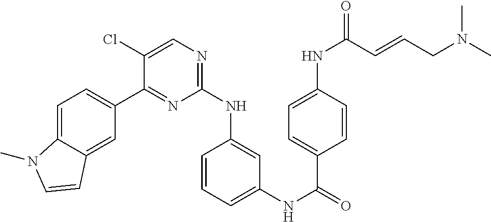 | Compound 179 |
| 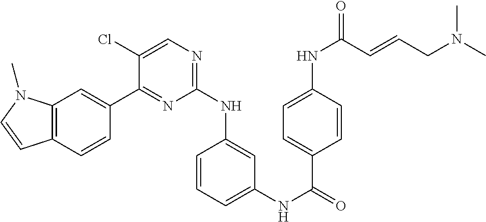 [SMILES: CN(C)C/C=C/C(=O)Nc1ccc(C(=O)Nc2cccc(Nc3ncc(Cl)c(-c4ccc5ccn(C)c5c4)n3)c2)cc1] | Compound 180 |
| 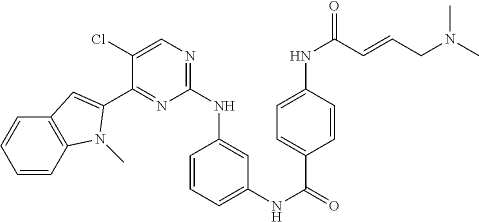 [SMILES: CN(C)C/C=C/C(=O)Nc1ccc(C(=O)Nc2cccc(Nc3ncc(Cl)c(-c4cc5ccccc5n4C)n3)c2)cc1] | Compound 181 |
| 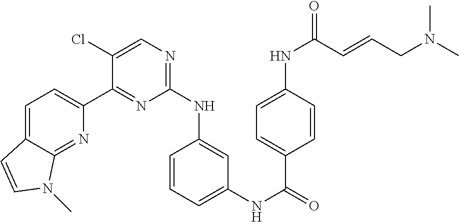 [SMILES: CN(C)C/C=C/C(=O)Nc1ccc(C(=O)Nc2cccc(Nc3ncc(Cl)c(-c4ccc5ccn(C)c5n4)n3)c2)cc1] | Compound 182 |
| 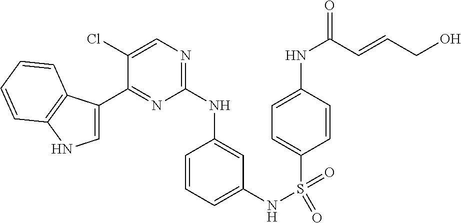 [SMILES: O=C(/C=C/CO)Nc1ccc(S(=O)(=O)Nc2cccc(Nc3ncc(Cl)c(-c4c[nH]c5ccccc45)n3)c2)cc1] | Compound 183 |
| 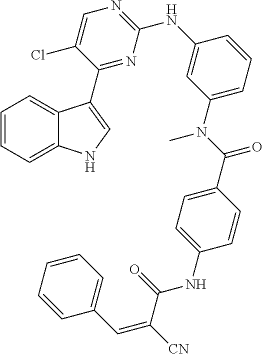 [SMILES: CN(C(=O)c1ccc(NC(=O)/C(C#N)=C\c2ccccc2)cc1)c1cccc(Nc2ncc(Cl)c(-c3c[nH]c4ccccc34)n2)c1] | Compound 184 |
| 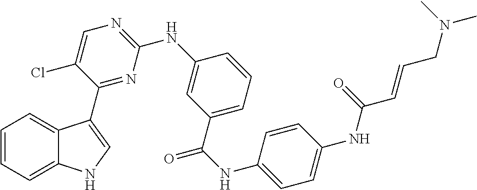 [SMILES: CN(C)C/C=C/C(=O)Nc1ccc(NC(=O)c2cccc(Nc3ncc(Cl)c(-c4c[nH]c5ccccc45)n3)c2)cc1] | Compound 185 |
| 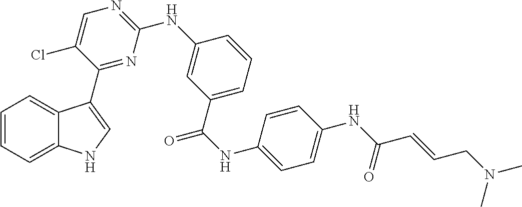 [SMILES: CN(C)C/C=C/C(=O)Nc1ccc(NC(=O)c2cccc(Nc3ncc(Cl)c(-c4c[nH]c5ccccc45)n3)c2)cc1] | Compound 186 |

Also contemplated herein are derivatives one or more compounds in Table 1 (i.e., compound 1 to compound 186).

Other exemplary CDK7 inhibitors can be found in e.g., US2017/0174692; WO2015/154038; US2016/0264552; US2016/0264551; US2016/0264554; US2015/122323; US2017/0183355; US2017/0174692; or WO2016/160617, the contents of each of which are incorporated herein by reference in their entireties. In particular, it is specifically contemplated herein that the methods and compositions described herein use a CDK7 inhibitor as described in US2017/0183355, the contents of which are incorporated herein by reference in their entirety.

In one embodiment, the CDK7 inhibitor comprises SY-1365.

A CDK7 inhibitor as described herein can be either a covalent or non-covalent inhibitor of CDK7.

In one embodiment, an agent inhibits the level and/or activity of CDK7 by at least 10%, by at least 20%, by at least 30%, by at least 40%, by at least 50%, by at least 60%, by at least 70%, by at least 80%, by at least 90%, at least 95%, at least 99%, or even 100% (e.g., no detectable CDK7 activity as assessed by measuring phosphorylation of RNA Pol II at Ser5 and Ser7) as compared to an appropriate control. As used herein, an “appropriate control” refers to the level and/or activity of CDK7 prior to administration of the agent, or the level and/or activity of CDK7 in a population of cells that was not in contact with the agent.

The agent may function directly in the form in which it is administered. Alternatively, the agent can be modified or utilized intracellularly to produce a product that inhibits CDK7, such as introduction of a nucleic acid sequence into the cell and its transcription resulting in the production of the nucleic acid and/or protein inhibitor of CDK7. In some embodiments, the agent is any chemical, entity or moiety, including without limitation synthetic and naturally-occurring non-proteinaceous entities

In various embodiments, the agent that inhibits CDK7 is an antibody or antigen-binding fragment thereof, or an antibody reagent that is specific or selective for CDK7. Where CDK7 is an intracellular factor, an antibody or fragment thereof may be more effective if either modified to cross the cell membrane, e.g., as delivered by a liposome, or for example, by expression within the cell, e.g., from a viral or other vector. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects described herein, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits CDK7 is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the agent that inhibits CDK7 is an antisense oligonucleotide. As used herein, an “antisense oligonucleotide” refers to a synthesized nucleic acid sequence that is complementary to a target DNA or mRNA sequence. Antisense oligonucleotides are typically designed to block expression of a DNA or RNA target by binding to the target and halting expression at the level of transcription, translation, or splicing. Antisense oligonucleotides are generally designed to hybridize under cellular conditions to a gene, e.g., the CDK7 gene, or to its transcript. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits CDK7 may comprise at least 5, at least 10, at least 15, at least 20, at least 25, at least 30, or more bases complementary to a portion of the coding sequence of the human CDK7 gene (e.g., NCBI Gene ID: 1022), respectively.

In one embodiment, the agent inhibits CDK7 by RNA inhibition or interference. The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but not limited to, siRNA, shRNA, endogenous microRNA and artificial microRNA. For instance, it includes sequences previously identified as siRNA, regardless of the mechanism of down-stream processing of the RNA.

In some embodiments of any of the aspects, the inhibitory nucleic acid is an inhibitory RNA (iRNA). The iRNA can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. CDK7. In some embodiments of any of the aspects, the agent is siRNA that inhibits CDK7 activity and/or expression.

One skilled in the art can design siRNA, shRNA, or miRNA to target CDK7, e.g., using publically available design tools. siRNA, shRNA, or miRNA can be synthetically made or expressed from a vector. Commercial sources include companies such as Dharmacon (Lafayette, Colo.) and Sigma Aldrich (St. Louis, Mo.), among others.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the methods and compositions described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits CDK7 expression and/or activity. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. An miRNA can be encoded by a nucleic acid that is expressed in the cell, e.g., from naked DNA, or can be encoded by a nucleic acid that is contained within a vector.

The agent may result in gene silencing of the target gene (e.g., CDK7), such as with an RNAi molecule (e.g. siRNA or miRNA). This entails a decrease in the mRNA level in a cell for a target by at least about 5%, at least about 10%, at least about 20%, at least about 30%, at least about 40%, at least about 50%, at least about 60%, at least about 70%, at least about 80%, at least about 90%, at least about 95%, at least about 99%, or even about 100% (i.e., below detectable limits by standard miRNA assay detection methods) of the mRNA level found in the cell without the presence of the agent. One skilled in the art will be able to readily assess whether the siRNA, shRNA, or miRNA effectively targets e.g., CDK7, for downregulation, for example by transfecting the siRNA, shRNA, or miRNA into cells and detecting the levels of a gene product (e.g., CDK7) found within the cell via western-blotting.

The agent may be contained in or expressed by a desired vector. Many such vectors useful for transferring exogenous genes into target mammalian cells are available. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a CDK7 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and/or proteins and as appropriate, secreting proteins, including where applicable, but not limited to, for example, transcription, transcript processing, translation and protein folding, modification and processing. “Expression products” include RNA transcribed from a gene and processing derivatives thereof, such as siRNA, shRNA, miRNA, etc., and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The vectors can be episomal, e.g. plasmids, virus-derived vectors such as cytomegalovirus, adenovirus, etc., or can be integrated into the target cell genome, through homologous recombination or random integration, e.g. for retrovirus-derived vectors such as MMLV, HIV-1, ALV, etc. In some embodiments, combinations of retroviruses and an appropriate packaging cell line may also find use, where the capsid proteins will be functional for infecting the target cells. Commonly used retroviral vectors are “defective”, i.e. unable to produce viral proteins required for productive infection. Replication of the vector requires growth in the packaging cell line.

Integrating vectors, such as retroviral vectors, lentiviral vectors, hybrid adenoviral vectors, and herpes simplex viral vector are specifically contemplated for use in the methods described herein. Alternatively, non-integrative vectors (e.g., non-integrative viral vectors) can be used and can eliminate the risks posed by integrative retroviruses, as they do not incorporate their genome into the host DNA. Non-limiting examples of non-integrating viral vectors include Epstein Barr oriP/Nuclear Antigen-1 (“EBNA1”) vector, RNA Sendai viral vector, or an F-deficient Sendai virus vector. Another example of a non-integrative vector is a minicircle vector. Minicircle vectors are circularized vectors in which the plasmid backbone has been released leaving only the eukaryotic promoter and cDNA(s) that are to be expressed.

### Administration and Efficacy

Embodiments of the compositions and methods described herein comprise administering an inhibitor of CDK7 to a subject having or diagnosed as having tuberous sclerosis complex or a secondary disease of TSC.

In some embodiments, the methods described herein comprise administering an effective amount of a composition described herein to a subject in order to alleviate a symptom of tuberous sclerosis complex or a sign or symptom thereof. As used herein, “alleviating a symptom” is reducing or ameliorating any condition or symptom associated with TSC (e.g., seizure frequency or severity, cardiac arrhythmia, cognitive decline, kidney failure, hospitalizations, loss of confidence, poor quality of life etc.). As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local, delivered directly to one or more TSC tumors, or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially in animal model assays as known in the art or as described in the Examples herein. Also, a dose can be formulated in animal models (e.g., TSC animal models such as TSC1 or TSC2 knockout mice) to achieve a circulating plasma concentration range that includes the IC50 (i.e., the concentration of the active agent which achieves a half-maximal inhibition of symptoms) as determined in an appropriate animal model. Levels in plasma can be measured, for example, by high performance liquid chromatography. The effects of any particular dosage can be monitored by a suitable bioassay, e.g., in cells or animal models of tuberous sclerosis complex, among others. The dosage can be determined by a physician and adjusted, as necessary, to suit observed effects of the treatment.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, where appropriate or desired, controlled-release parenteral dosage forms can be prepared for administration of a patient, including, but not limited to, DUROS®-type dosage forms and dose-dumping.

Suitable vehicles that can be used to provide parenteral dosage forms of a composition as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, and particularly where, for example, the permeability of a barrier is to be reduced or decreased for therapeutic benefit, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cheng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, and the presence of certain enzymes and other physiological conditions or compounds, among others.

A variety of known controlled- or extended-release dosage forms, formulations, and devices can be adapted for use with the compositions, agents or salts thereof in the instant disclosure. Controlled-release formulations can be used to control, for example, a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In one embodiment, the agent described herein is used as a monotherapy. The methods described herein can further comprise administering a second agent and/or treatment to the subject, e.g. as part of a combinatorial therapy. For example, an inhibitor of CDK7 (e.g., THZ1 or SY-1365) can be administered in combination with rapamycin or an analog or derivative thereof that has activity against mTORC1.

Administered “in combination,” as used herein, means that two (or more) different treatments are delivered to the subject during the course of the subject's affliction with the disorder, e.g., the two or more treatments are delivered after the subject has been diagnosed with the disorder (e.g., tuberous sclerosis complex) and before the disorder has been cured or eliminated or treatment has ceased for other reasons. In some embodiments, the delivery of one treatment is still occurring when the delivery of the second begins, so that there is overlap in terms of administration. This is sometimes referred to as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

There are currently no therapeutics available for the treatment of TSC, however the methods and compositions described herein can be combined with any agent that can provide symptomatic relief of TSC-associated conditions. Non-limiting examples of agents that target conditions/symptoms associated with TSC that can be used in combination with inhibitors of CDK7 include: rapamycin and analogs thereof such as everolimus, sirolimus and temsirolimus, among others; immunomodulators (e.g., a corticosteroid (e.g., betamethasone, budesonide, cortisone, dexamethasone, hydrocortisone, methylprednisolone, prednisolone, and/or prednisone)); anti-hypertensive agents (e.g., diuretics, adrenergic receptor antagonists, adrenergic receptor agonists, calcium channel blockers, ACE inhibitors, angiotensin II receptor antagonists aldosterone antagonists, vasodilators, renin inhibitors, and combinations thereof); anti-seizure medications (e.g., acetazolamide, carbamazepine, clobazam, clonazepam, eslicarbezepine acetate, ethosuximide, gabapentin, lacosamide, lamotrigine, levetriacetam, nitrazepam, oxcarbazepine, perampanel, piracetam, phenobarbital, phenytoin, pregabalin, primidone, rufinamide, sodium valproate, stiripentol, tiagabine, topiramate, vigabatrin, zonisamide, etc.); mood stabilizers (e.g., lithium); antipsychotics (e.g., aripiprazole, risperidone, olanzapine, quetiapine, asenapine, paliperidone, ziprasidone, lurasidone etc.); anti-depressants (e.g., selective serotonin reuptake inhibitors (e.g., citalopram etc.); serotonin-norepinephrine reuptake inhibitors; serotonin modulators and stimulators (e.g., vorioxetine); serotonin antagonists and reuptake inhibitors (e.g., trazodone, nefazodone etc.); norepinephrine reuptake inhibitors; tricyclic anti-depressants; tetracyclic anti-depressants; monoamine oxidase inhibitors etc.); and anti-arrhythmic agents (e.g., sodium channel blockers, beta blockers, potassium-channel blockers, adenosine, digitalis, atropine etc.).

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., anti-seizure, anti-arrhythmic or mood stabilizing effects) is lower (e.g., at least 20%, at least 30%, at least 40%, or at least 50% lower) than the amount or dosage of each agent individually required to achieve the same therapeutic effect.

In certain embodiments, an effective dose of a composition as described herein can be administered to a patient once. In certain embodiments, an effective dose of a composition can be administered to a patient repeatedly. A unit dosage form for a given composition or agent can be a preparation including the amount necessary to achieve a desired effective concentration in one or more tissues of the body in a single dose. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

In one embodiment, the CDK7 inhibitor used in the methods and compositions described herein is SY-1365, which is a covalent inhibitor of CDK7 with high potency (i.e., enzymatic IC50 ˜22 nm; cellular IC50 ˜20 nM) and high selectivity for CDK7 over other CDKs (e.g., CDK9). In some embodiments, the dose of SY-1365 administered to a subject in need thereof is at least 20 nM, at least 22 nM, at least 25 nM, at least 50 nM, at least 100 nM, at least 150 nM, at least 200 nM, at least 250 nM, at least 300 nM, at least 350 nM, at least 400 nM, at least 450 nM, at least 500 nM or higher. In other embodiments, the dose of SY-1365 is within the range of 20 nM-500 nM, 20 nM-400 nM, 20 nM-300 nM, 20 nM-200 nM, 20 nM-100 nM, 20 nM-50 nM, 50-500 nM, 100-500 nM, 200-500 nM, 300-500 nM, 400-500 nM, 50 nM-100 nM, 25 nM-75 nM, 100-200 nM, or any range therebetween.

In certain embodiments, the dose of SY1365 is determined based on body weight, for example, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg at least 100 mg/kg or more. Non-limiting examples of suitable dose ranges include 10-100 mg/kg, 10-90 mg/kg, 10-80 mg/kg, 10-70 mg/kg, 10-60 mg/kg, 10-50 mg/kg, 10-40 mg/kg, 10-30 mg/kg, 10-20 mg/kg, 90-100 mg/kg, 80-100 mg/kg, 70-100 mg/kg, 60-100 mg/kg, 50-100 mg/kg, 40-100 mg/kg, 30-100 mg/kg, 20-100 mg/kg, 20-50 mg/kg, 25-75 mg/kg, 40-60 mg/kg, 60-80 mg/kg, or any range therebetween.

In other embodiments, the CDK7 inhibitor used in the methods and compositions described herein is THZ1 or a derivative thereof. The IC50 of THZ1 is ˜3.2, indicating that it is highly potent. In certain embodiments, the dose of THZ1 is determined based on body weight, for example, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg at least 100 mg/kg or more. Non-limiting examples of suitable dose ranges include 10-100 mg/kg, 10-90 mg/kg, 10-80 mg/kg, 10-70 mg/kg, 10-60 mg/kg, 10-50 mg/kg, 10-40 mg/kg, 10-30 mg/kg, 10-20 mg/kg, 90-100 mg/kg, 80-100 mg/kg, 70-100 mg/kg, 60-100 mg/kg, 50-100 mg/kg, 40-100 mg/kg, 30-100 mg/kg, 20-100 mg/kg, 20-50 mg/kg, 25-75 mg/kg, 40-60 mg/kg, 60-80 mg/kg, or any range therebetween. In some embodiments, the dose of THZ1 administered to a subject in need thereof is at least 20 nM, at least 22 nM, at least 25 nM, at least 50 nM, at least 100 nM, at least 150 nM, at least 200 nM, at least 250 nM, at least 300 nM, at least 350 nM, at least 400 nM, at least 450 nM, at least 500 nM or higher. In other embodiments, the dose of SY-1365 is within the range of 20 nM-500 nM, 20 nM-400 nM, 20 nM-300 nM, 20 nM-200 nM, 20 nM-100 nM, 20 nM-50 nM, 50-500 nM, 100-500 nM, 200-500 nM, 300-500 nM, 400-500 nM, 50 nM-100 nM, 25 nM-75 nM, 100-200 nM, or any range therebetween.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of effect can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the composition, its potency, and the extent to which symptoms, markers, or indicators of a condition described herein are desired to be reduced, for example the degree of CDK7 inhibition. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. CDK17 expression and/or activity to a detectable agent as described herein. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., reduced tuber or hamartoma number of size, or symptoms of TSC such as seizure frequency or severity). It is well within the ability of one skilled in the art to monitor efficacy of administration and/or treatment by measuring any one of such parameters, or any combination of parameters. Efficacy can be assessed in animal models of a condition described herein, for example animal models of TSC, such as TSC1 or TSC2 deficient mice. When using an experimental animal model, efficacy of treatment is evidenced when a statistically significant change in a marker is observed.

In vitro and animal model assays are provided herein which allow the assessment of a given dose of a composition. The efficacy of a given dosage combination can also be assessed in an animal model, e.g. a murine xenograft model or a TSC-deficient mouse model such as a TSC2+/−AJ mouse model as described in the Examples herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### Examples

### Example 1: Use of THZ1 and Other CDK7 Inhibitors as Therapeutic Agents in Tuberous Sclerosis Complex (TSC)

Tuberous sclerosis complex (TSC) is caused by germline loss-of-function mutations in TSC1 or TSC2. Bi-allelic loss of either TSC1 or TSC2 occurs in TSC tumors, leading to inactivation of the TSC1/TSC2 protein complex, and activation of mTORC1 with multiple downstream effects on anabolism and cell growth. Rapalogs, mTORC1 inhibitors, are effective cytostatic agents for the treatment of TSC, but lifelong therapy appears to be required for continuing benefit. Therapies for TSC that induce a selective cytocidal response in TSC-deficient cells are not currently clinically available, and are highly desirable for TSC patients.

In this study, it was hypothesized that since mTORC1 activation enhances RNA transcription, this enhanced transcription might create a therapeutic vulnerability. In addition, it was hypothesized that rapamycin treatment of TSC-deficient cells would lead to complex compensatory effects on transcription, and that synergy might be seen in co-treatment with a transcriptional inhibitor. RNA polymerase II, the polymerase responsible for RNA transcription in mammalian cells, contains an extended C-terminal domain (CTD), which is subject to intricate phosphorylation and dephosphorylation during transcript initiation, elongation, and termination. Cyclin-dependent kinase 7 (CDK7) plays a critical role in the phosphorylation of Ser5 and Ser7 of a heptapeptide repeat in the RNA Pol II CTD. A kinase inhibitor screen focused on CDK7 identified THZ1 as a selective covalent inhibitor, and subsequent studies showed it had cytocidal effects for several cancer types both in vitro and in vivo in mouse models. Thus, it was hypothesized that THZ1 may also have selective cytocidal effects on TSC1/TSC2-deficient cells with hyperactive mTORC1 in comparison to controls. THZ1 derivatives, such as SY-1365 (Syros Pharmaceuticals), have improved pharmacokinetic properties compared to THZ1, and are in early human clinical trials.

Multiple studies were performed that support these hypotheses. Most studies to date have been performed on two pairs of cell lines: (i) HCV29, a TSC1 null human bladder cancer cell line, and a corresponding TSC1 addback derivative (TSC1-HCV29), and (ii) 621-101, a TSC2 null angiomyolipoma cell line, and the TSC2 addback derivative 21-103.

### CDK7 Inhibition by THZ1 Selectively Inhibits the Growth of TSC-Deficient Cells.

In a standard 96-well plate assay, THZ1 showed selective inhibition of proliferation of HCV29 vs. TSC1-HCV29, and 621-101 cells vs. 621-103 (expressing TSC2) (FIG. 1). The IC50 of each of HCV29 and 621-101 cells was ˜30 nM, while it was 6-8 fold higher for the addback lines.

### THZ1 Selectively Induces Cell Cycle Arrest and Apoptosis in TSC-Deficient Cells.

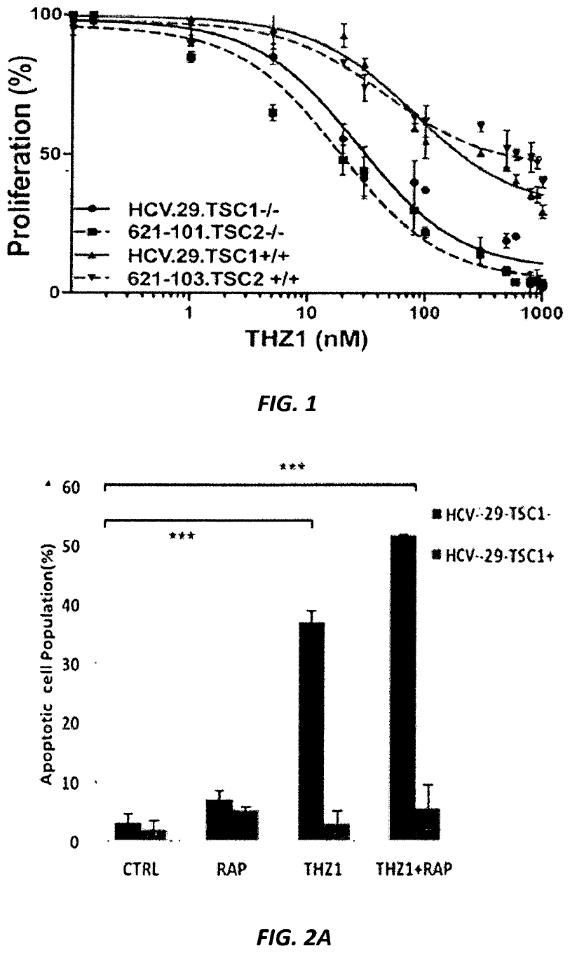

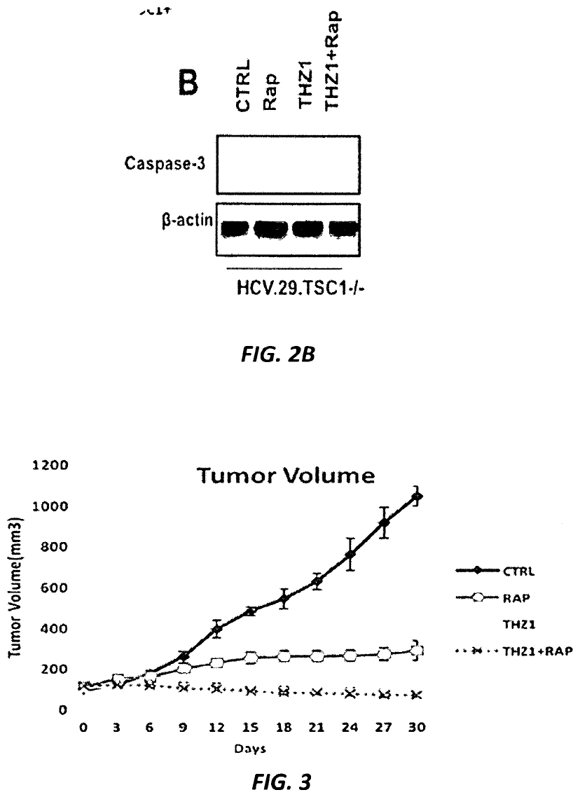

Cell cycle flow cytometry analysis demonstrated that THZ1 (30 nM, 24 h) arrested 21% of HCV29 cells in G2/M-phase vs. 5.5% of TSC1-HCV29 cells. Flow cytometry of cells treated with 30 nM THZ1 for 72 h, and stained with propidium iodide and Annexin V, showed that 37% of HCV29 cells were Annexin V+ vs. 2% of TSC1-HCV20 cells (FIG. 2A). Furthermore, cleaved Caspase 3 was markedly increased in the THZ1-treated HCV29 cells (FIG. 2B). In addition, co-treatment with rapamycin and THZ1 showed synergistic effects with an increase in the apoptotic cell fraction for HCV29 cells.

### THZ1 Treatment of HCV29 Cells Induces Increased ROS Levels and Depletion of Glutathione.

To explore the mechanism of cell death induction, ROS levels were compared among HCV29 and TSC1-HCV29 cells untreated, or treated with THZ1 30 nM or rapamycin 20 nM or both drugs. ROS levels were increased 2.5-fold and 3.6-fold respectively in the THZ1-treated and THZ1/rap-treated cells, whereas no change was seen with rapamycin treatment alone. Steady state metabolite analysis of HCV29 treated with or without 30 nM THZ1 for 48 h (n=3 replicates for each condition), showed that six metabolites were reduced by 4-fold or greater in the THZ1-treated cells, including S-adenosyl-L-homocysteine and glutathione (GSH) with 87% and 78% reduction in levels, respectively. Glutathione disulfide (GSS) levels were also reduced by 65% in the THZ1-treated cells. Given previous evidence of a critical dependency in mTORC1-driven tumor cells on glutaminase activity to produce glutathione and sensitivity to reduction in glutathione levels by RNAi-mediated knockdown of GCLC, the inventors focused further on the marked reduction in glutathione seen with THZ1 treatment.

To determine directly whether GSH depletion was causing cell death in response to THZ1 treatment, HCV29 cells were treated with 30 nM THZ1, 1 mM GSH, or both. GSH co-treatment markedly reduced THZ1-induced HCV29 cell death, indicating that GSH reduction was required for growth inhibition and cell death.

THZ1 Treatment has Major Effects on Gene Expression that Cause Glutathione Depletion.

Previous studies with other cancer cell lines have shown that THZ1 treatment can cause selective loss of cancer-specific oncogene expression leading to tumor cell death. RNA-Seq was performed on HCV29 cells and TSC1-HCV20 cells treated with 0 (control), 30 nM and 100 nM THZ1 for 6 hours (n=2 replicates for each condition). Comparison of gene expression changes at the 30 nM THZ1 dose identified many genes with markedly different expression: e.g., 1128 genes showed a >5-fold lower expression in THZ1-treated HCV29 compared with THZ1− treated TSC1-HCV29 cells. In contrast, 90% of those gene showed similar levels (<1.5-fold up or down) in the two cell lines untreated. Pathway analysis using enrichr failed to identify a consistent pathway that was affected by THZ1, consistent with a broad effect on transcription. However, the inventors focused on expression of all genes involved in the synthesis of reduced glutathione. GSR (glutathione-disulfide reductase), GCLC (glutamate-cysteine ligase catalytic subunit), and GCLM (glutamate-cysteine ligase modifier subunit) showed a 86%, 82%, and 68% reduction (respectively) in mRNA levels in 30 nM THZ1-treated HCV29 cells, in comparison to 20 nM THZ1-treated TSC1-HCV29 cells. These data indicate that the mechanism of glutathione depletion in HCV29 cells in response to THZ1 is due at least in part to a selective effect on expression of these genes critical for production of reduced glutathione.

### THZ1 Inhibits Tumor Growth of TSC1-Deficient HCV29 Cells as Xenografts.

HCV29 xenograft tumors were induced in immune-deficient Foxn1^{nu} mice by injection of 10⁷ cells into each flank. After xenograft tumors reached ˜100 mm³ in size (˜30 days), mice were randomly assigned to treatment with placebo, THZ1 (10 mg/kg IP twice daily), rapamycin (3 mg/kg IP 3 days/week), or a combination of both for 30 days (n+5 mice and 10 flank tumors for each treatment group). Tumor size was monitored using calipers. Tumor growth rate was markedly reduced in mice treated with THZ1+/−rapamycin as compared to controls (FIG. 3). Furthermore, THZ1-treated tumors showed no regrowth after cessation of treatment for over 60 days (n+3 mice, 6 tumors). THZ1 treatment did not affect body weight or cause other apparent toxicities.

### Example 2

Throughout this example, the term “TSC-null” is used to refer to cell lines in which there is homozygous (complete) deletion of either TSC1 or TSC2; “TSC-addback” refers to cell lines in which TSC1/TSC2 loss is restored by expression of the protein through transfection.

### CDK7 Inhibition by THZ1 Selectively Targets the Viability of TSC-Deficient Cells

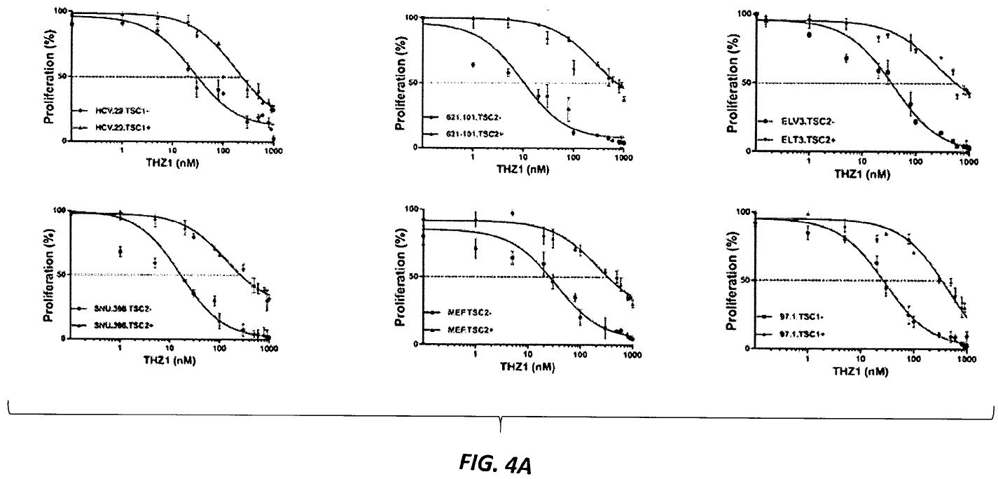

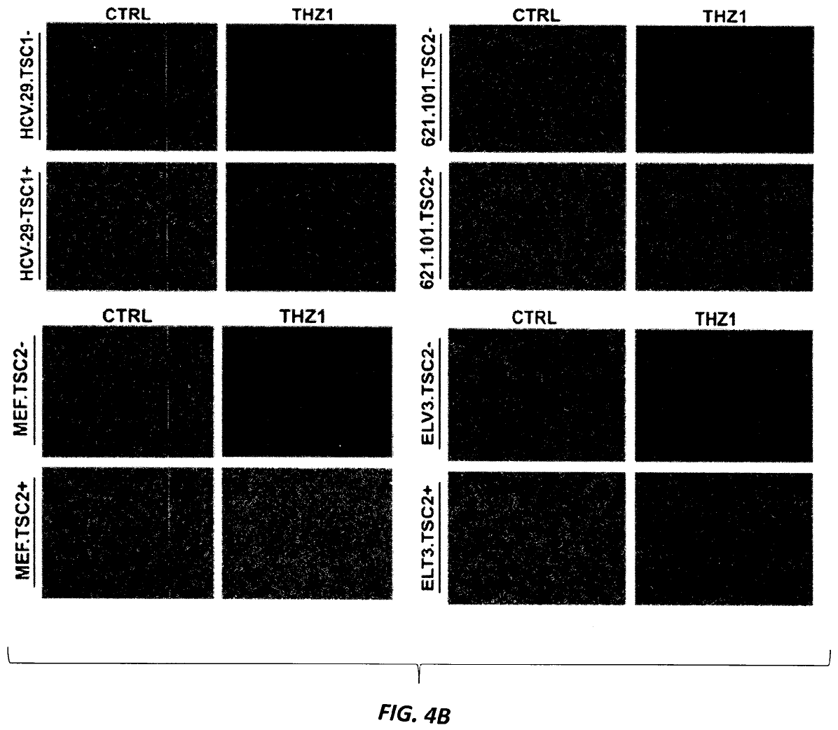

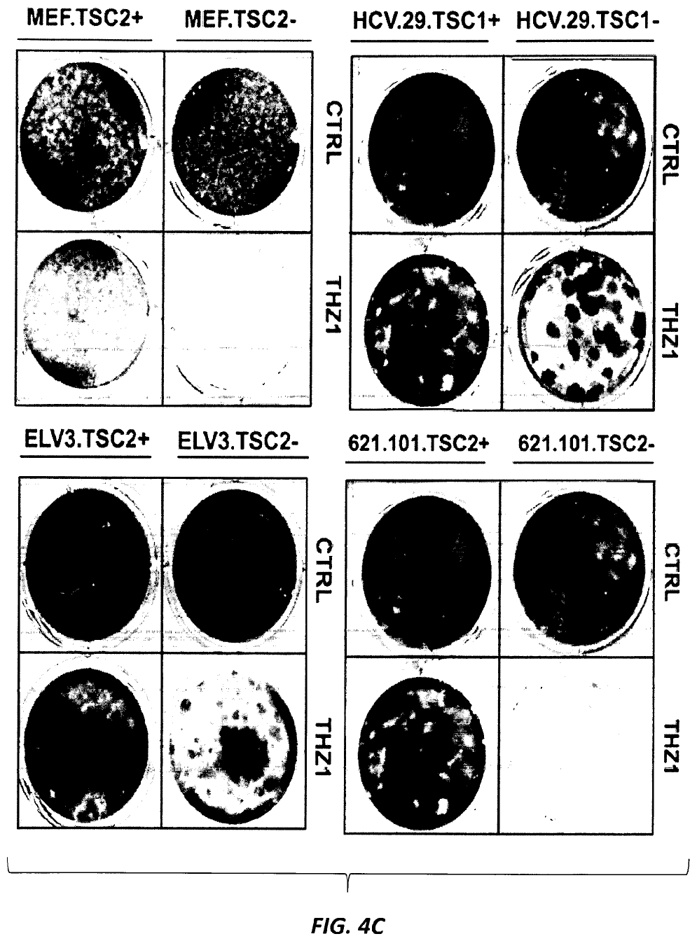

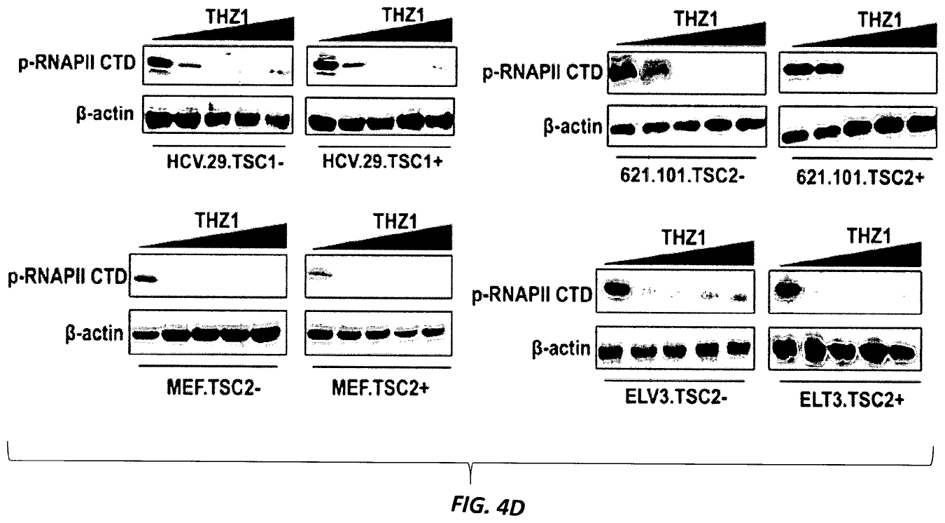

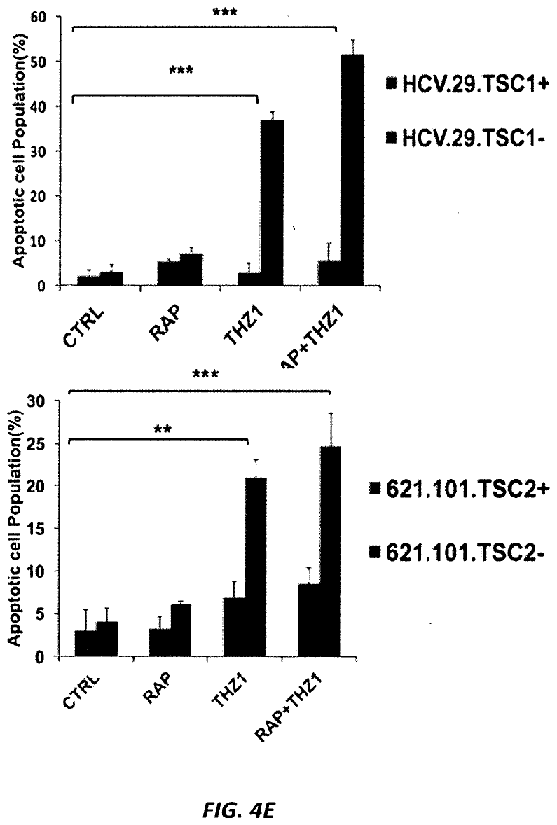

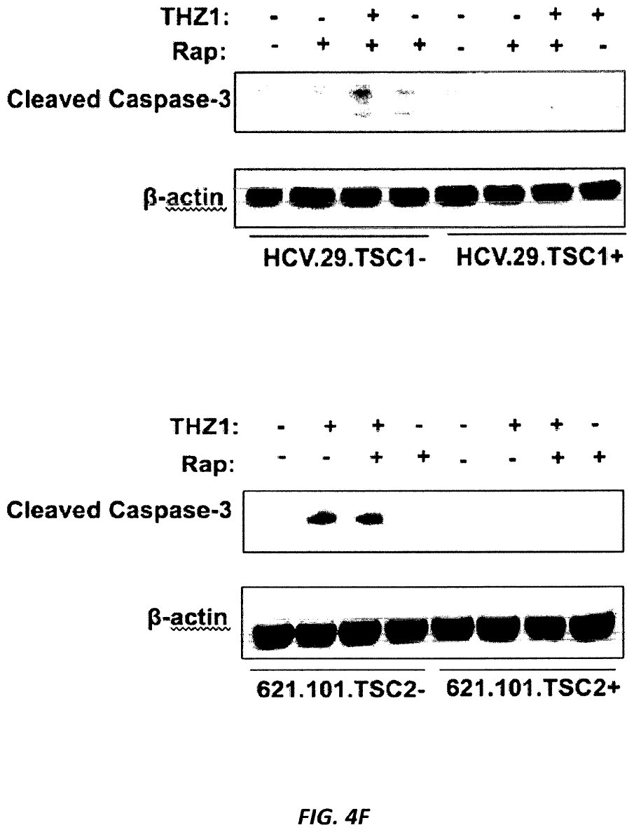

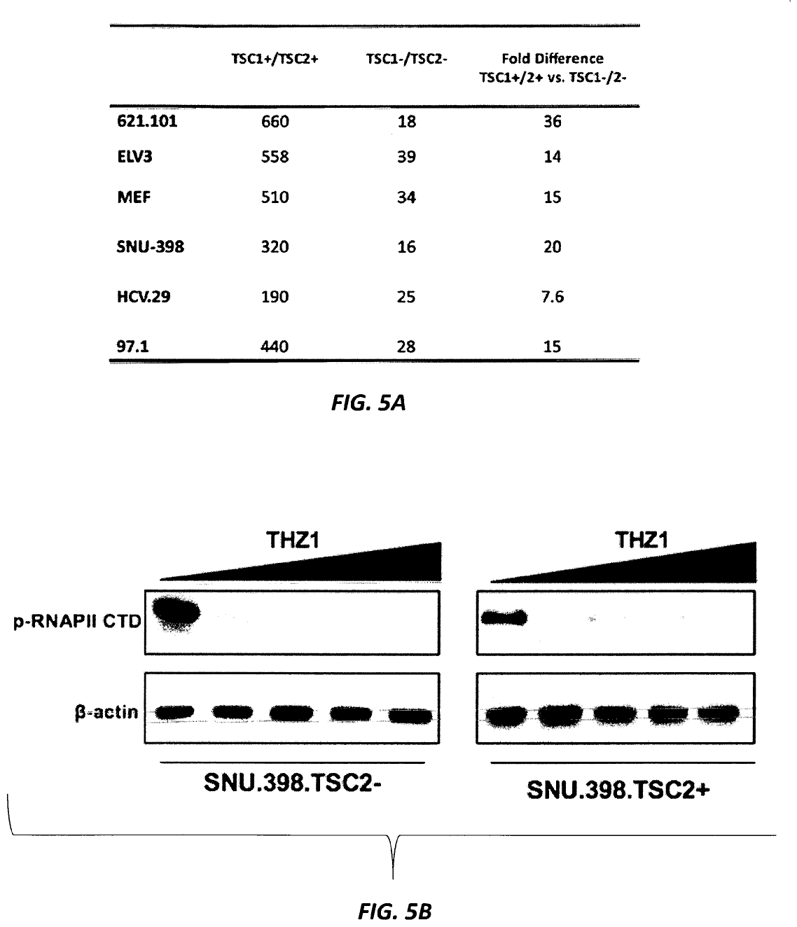

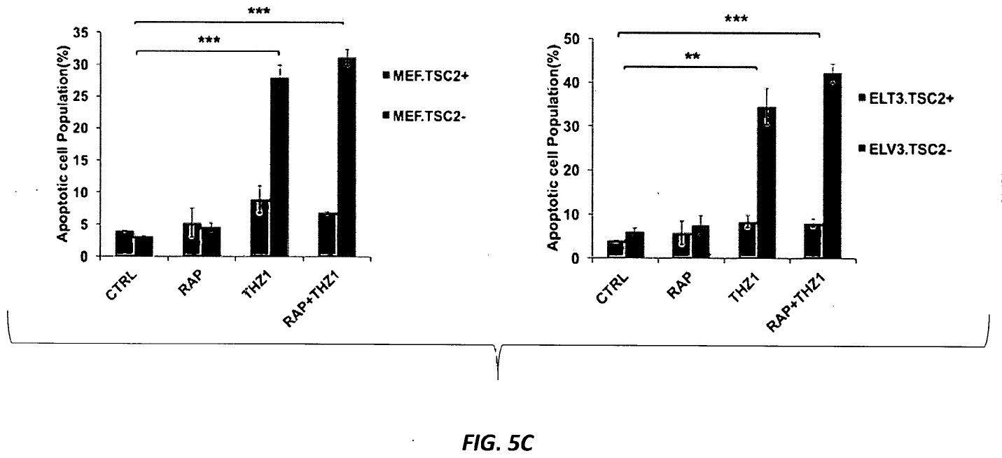

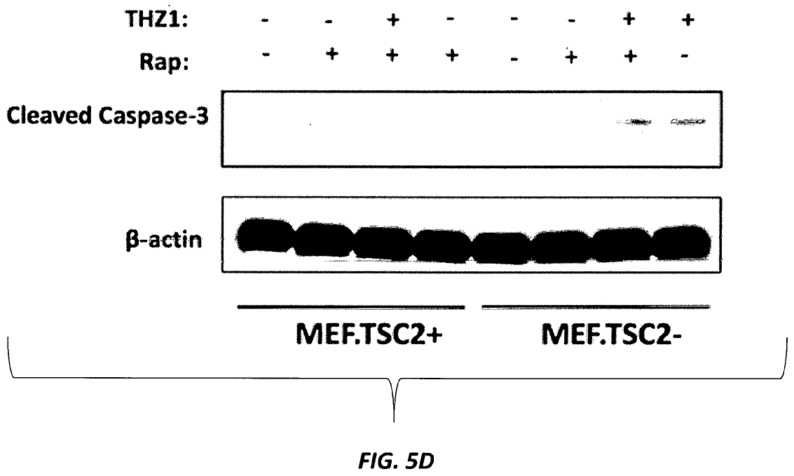

To investigate whether the proliferation of TSC-null cells is sensitive to CDK7 inhibition, TSC-null or TSC-addback cell lines were treated with increasing concentrations of THZ1. THZ1 showed selective inhibition of proliferation in TSC-null cells vs. TSC-addback cell lines (n=6 cell line pairs, 4 TSC2 null, 2 TSC1 null) in a standard 96 well plate growth assay (FIG. 4A). The IC50 of the TSC-null cell lines was 7-36-fold lower (median IC50 26.5 nM, range 16-39 nM) vs. the corresponding addback lines (median 475 nM, range 190-660 nM, FIG. 5A). Phase contrast imaging demonstrated that THZ1 treatment (30 nM, 72 hours) resulted in dramatic cell death in TSC-null cells compared to TSC-addback cells (FIG. 4B). Furthermore, apoptotic cell death was selectively induced by THZ1 treatment of TSC-null cells, as assessed by propidium iodide (PI) staining, and production of cleaved caspase 3, again in contrast to TSC-addback cells (FIGS. 4E, 4F and 5C, 5D). In addition, low dilution plating colony formation assays also showed that there was a marked reduction in colony formation in TSC-null cells treated with THZ1, in contrast to control TSC-addback lines (FIG. 4C). Together these data indicate that THZ1 induces cell death in a TSC-dependent manner.

In an effort to understand the mechanism of the TSC genotype specific effect of THZ1 inhibition of CDK7 on cell growth, it was examined whether CDK7 is inhibited equally in TSC-null and TSC-addback cells. TSC-null and TSC-addback cell lines displayed a similar dose-responsive reduction in RNAPolII CTD phosphorylation in response to THZ1 treatment (FIG. 4D, 5B), indicating that neither differential uptake of THZ1 nor differential inhibition of CDK7 explained the genotype-specific effect.

### TSC-Deficient Cells are Highly Dependent on CDK7 for Survival and Proliferation

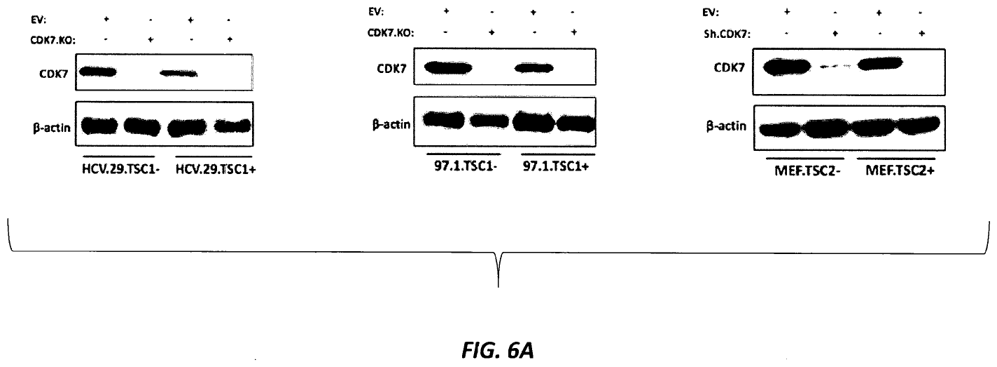

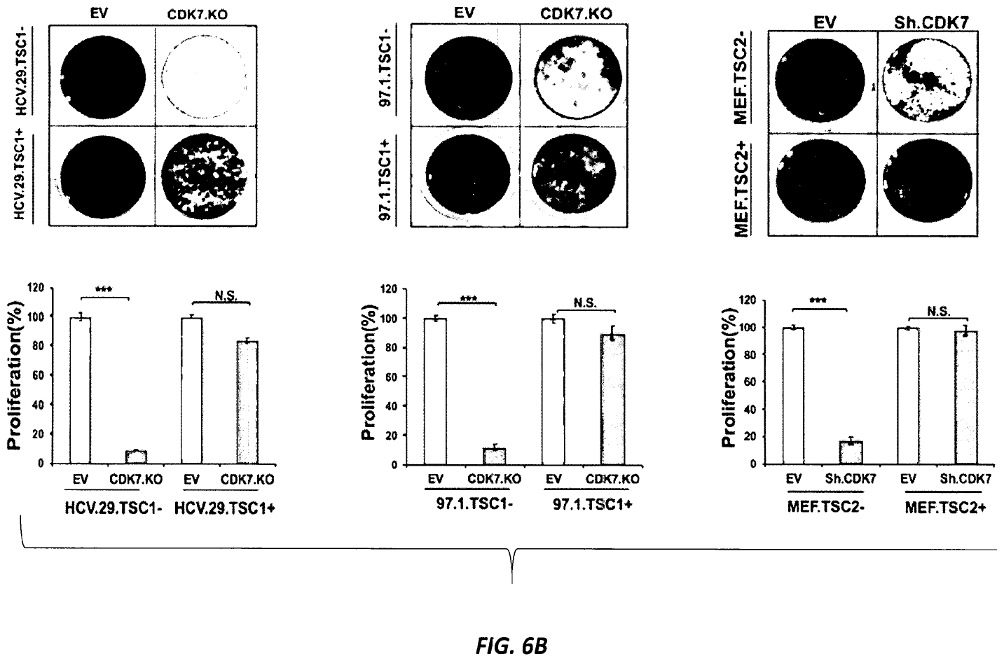

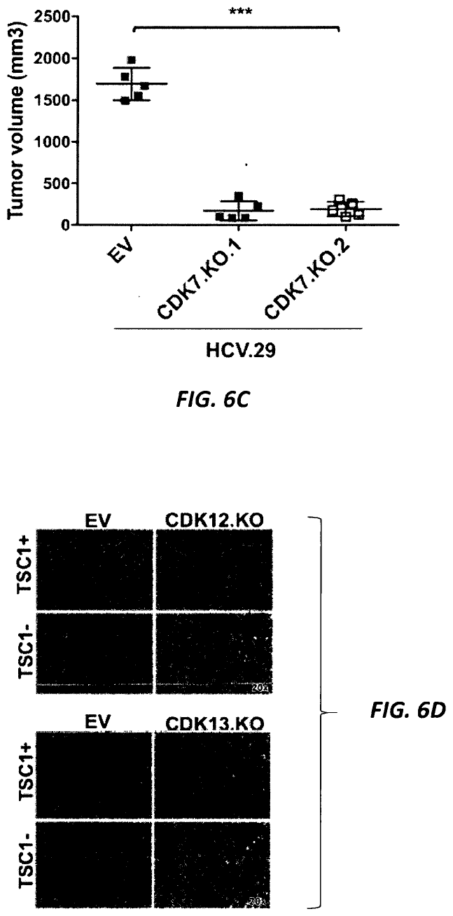

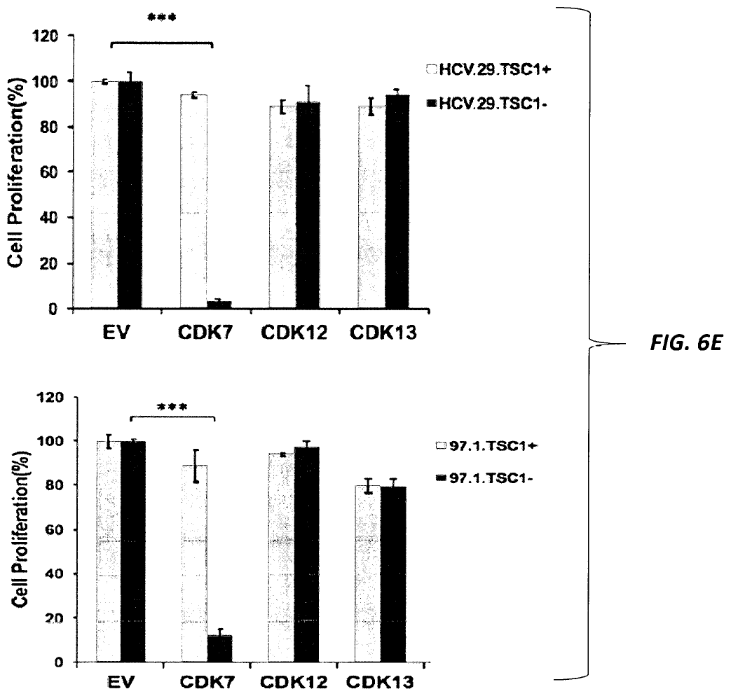

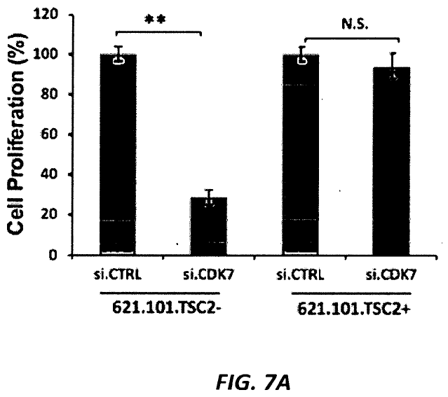

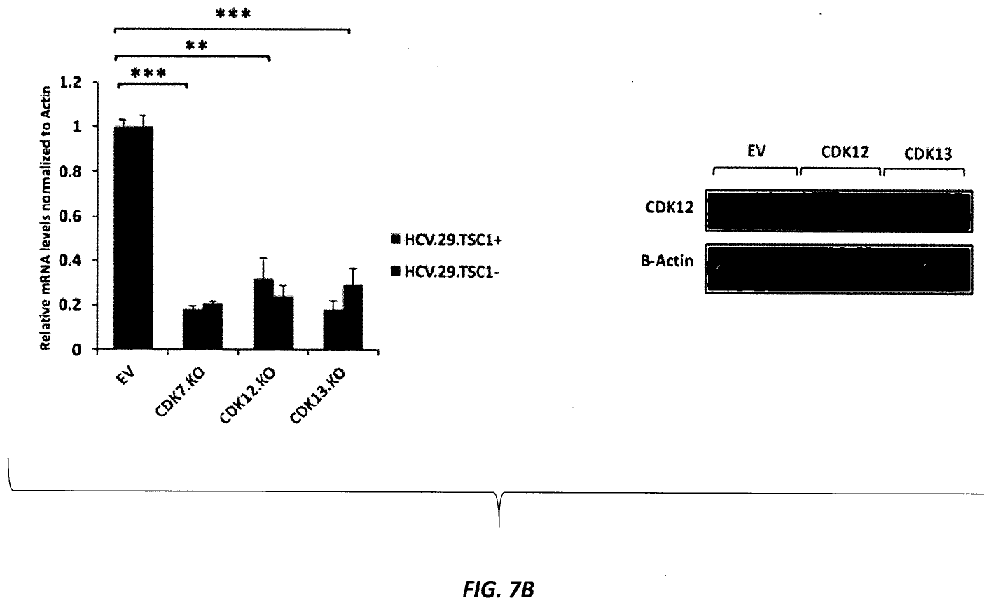

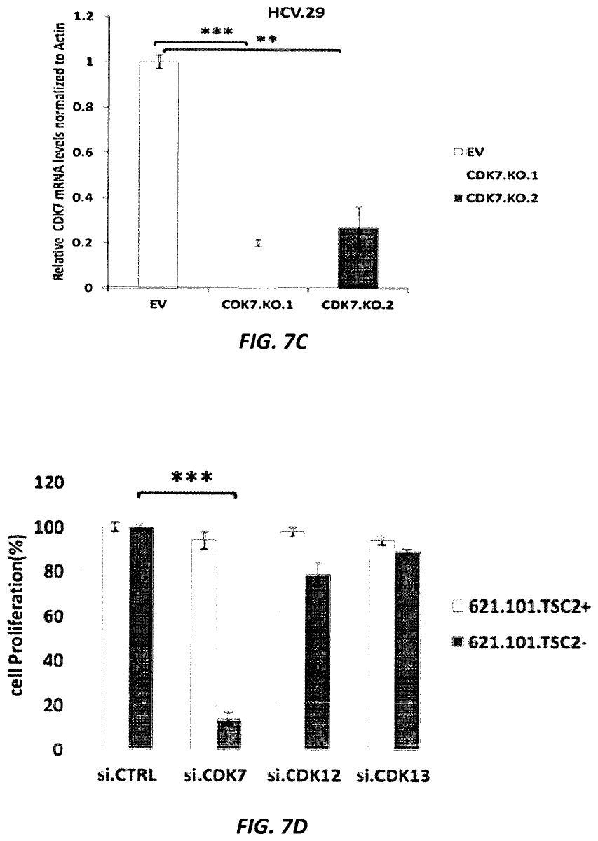

CDK7, and to a lesser extent CDK12/CDK13, are inhibited by THZ1 (Chipumuro et al., 2014; Christensen et al., 2014; Kwiatkowski et al., 2014). Previous studies in other cell systems have demonstrated that knockout of CDK7 inhibits cell survival, suggesting that it is the primary target of THZ1 in causing cell death. Here, to confirm that THZ1 is the critical pharmacological target of THZ1 in TSC-null cells, CRISPR/CAS9 was used to genetically knockout CDK7 gene in TSC1-null cells, both HCV29 and 97-1, and their addback derivatives. Immunoblot (FIG. 6A) and Q-RT-PCR assays (FIGS. 7B, 7C) confirmed a marked reduction in CDK7 expression, and both CDK7-KO TSC1-null cell lines showed a >90% reduction in proliferation, and near absence of growth in low dilution plating colony formation assays (FIG. 6B). Similarly, short hairpin RNA (shRNA) was used to decrease expression of CDK7 in Tsc2-null MEF cells, which demonstrated a significant reduction in expression of CDK7, with similar major effects on both proliferation and growth in low dilution plating colony formation assays (FIGS. 6A, 6B right). To investigate the growth properties of CDK7-KO cells in vivo, the CDK7-KO TSC1-null HCV29 cells were injected into the flanks of nude mice, and a near absence of xenograft formation was observed, in comparison to wild type controls in which robust xenograft growth occurred, necessitating mouse sacrifice at 51 days post-injection (FIG. 6C). Reduced CDK7 expression was confirmed in the xenograft tumor nodules of the CDK7-KO cells (FIG. 7C).

Since THZ1 has some inhibitory effects on CDK12 and CDK13 at higher doses (Chipumuro et al., 2014; Kwiatkowski et al., 2014), it was also examined whether those kinases contributed to the growth inhibition effect of THZ1. CRISPR/Cas9 was used to knockout both genes individually in the TSC1-null HCV29 and 97-1 cells (FIG. 7B). In contrast to knock out of CDK7, CDK12-KO and CDK13-KO derivative lines showed no significant reduction in growth and proliferation (FIGS. 6D, 7B, and 7D). Similarly, siRNA-mediated knockdown of CDK7, but not CDK12 or CDK13 in the TSC2-null 621-101 cell line, had significant effects on cell proliferation (FIG. 7D). Without wishing to be bound by theory, these data indicate that CDK7 is uniquely required for the survival and proliferation of TSC-null cells, and is the likely target of THZ1 in causing reduced cell growth and apoptosis of TSC-null cells.

### THZ1-Induced Decrease in Glutathione Levels is Required for Cell Death Induction in TSC-Deficient Cells

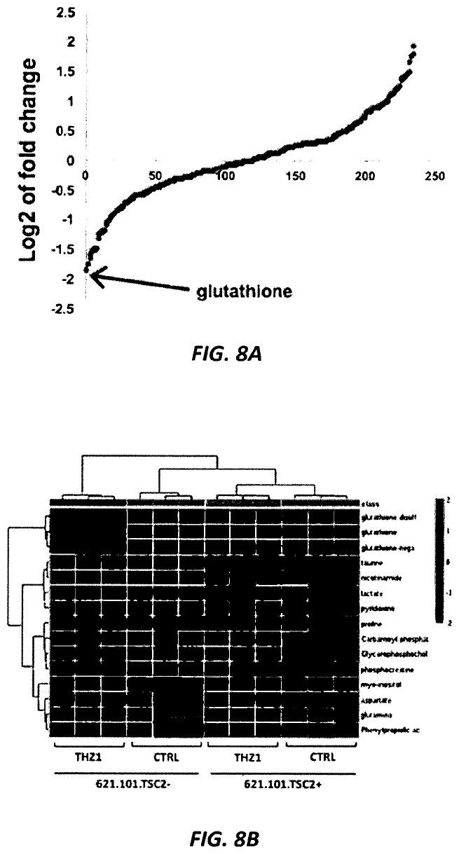

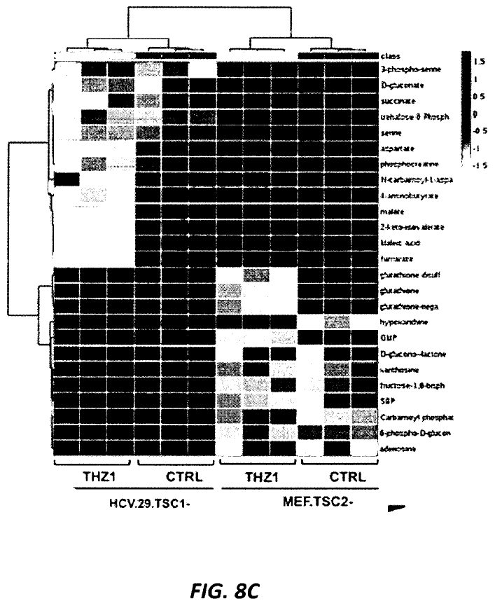

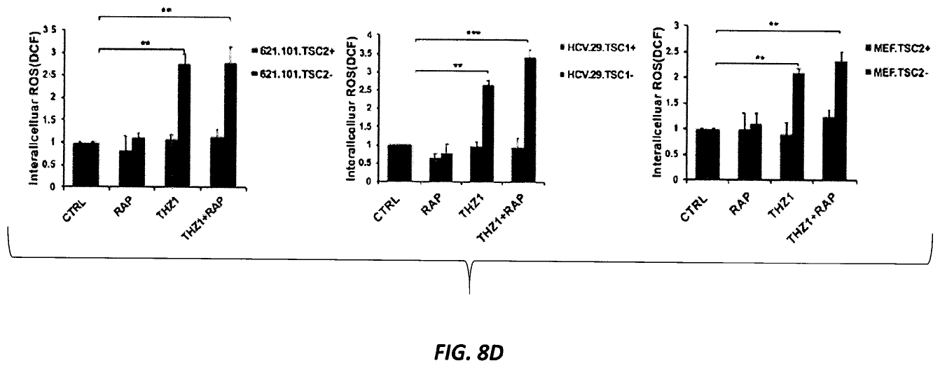

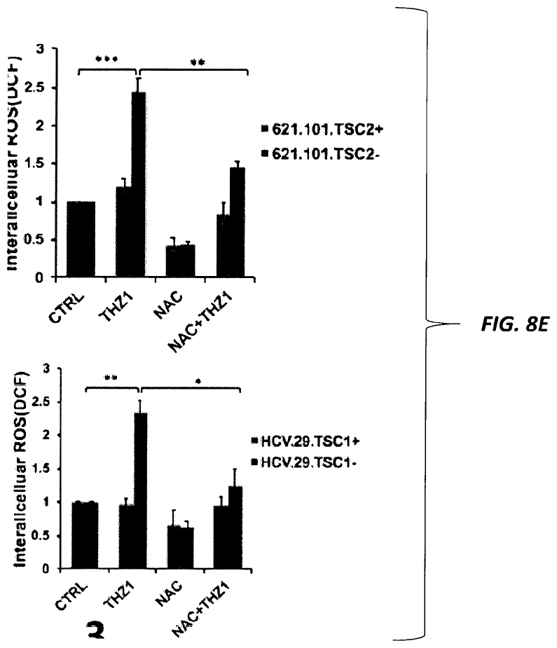

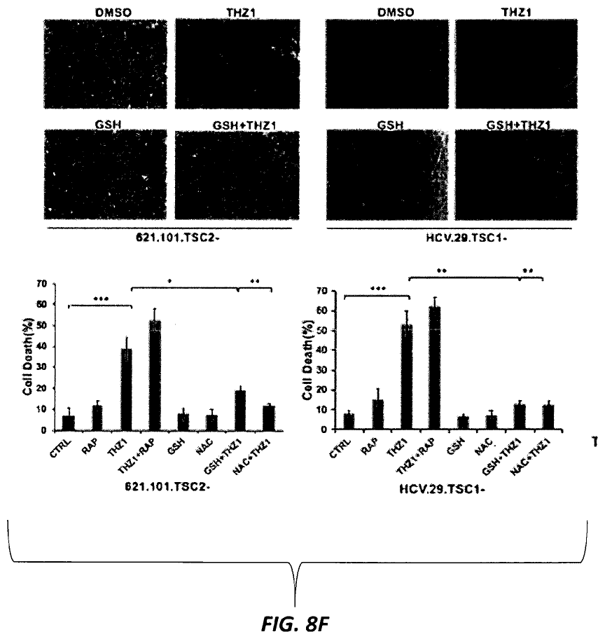

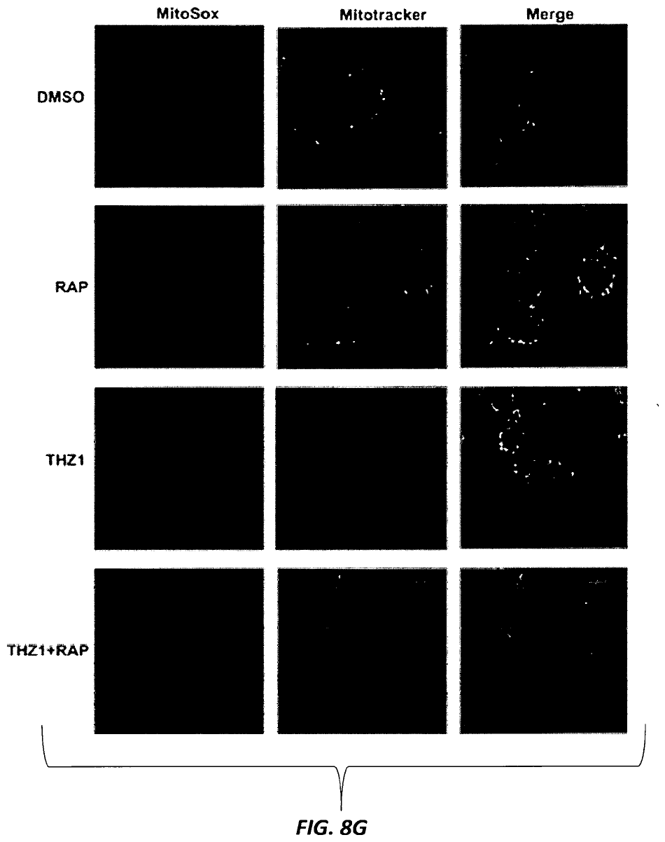

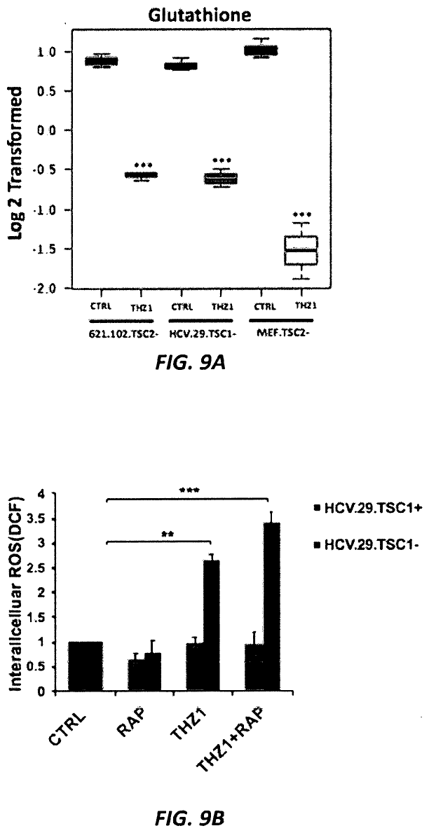

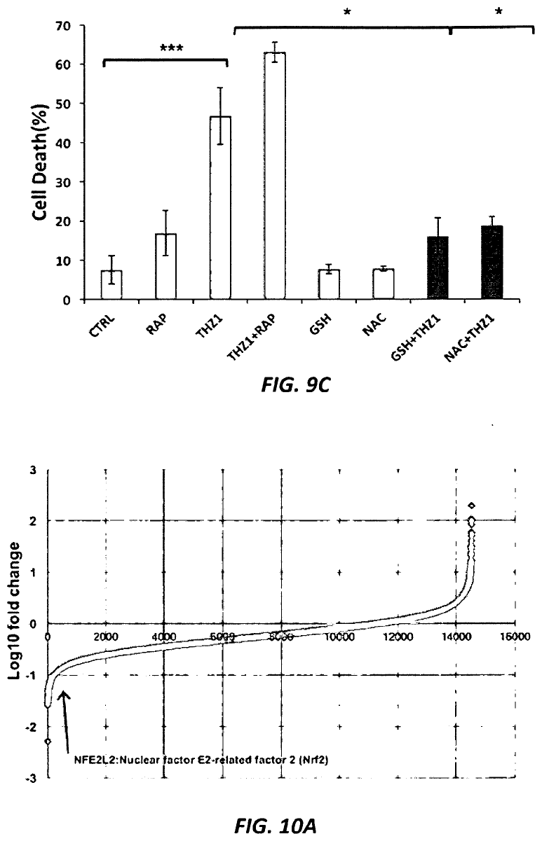

To investigate the mechanism by which THZ1 is selectively toxic to TSC-null cells, LC-MS/MS based metabolomics was used to profile metabolic changes following THZ1 treatment. After 6 hours of 30 nM THZ1 treatment, metabolites from the TSC-null cells was altered dramatically, and glutathione (GSH) was the metabolite that was decreased to the largest degree among 241 measured metabolites (FIG. 8A). Similar marked reductions in glutathione levels were seen for TSC2-null 621-101 cells, TSC1-null HCV29 cells and Tsc2-null MEFs (FIGS. 8B, 8C and 9A). As GSH is the major intracellular antioxidant protein, reactive oxygen species (ROS) were next measured in these cells. THZ1 treatment caused a marked increase in ROS levels in TSC-null cells in comparison to parallel TSC-addback cells (TSC2-null 621-101 cells, TSC1-null HCV29 cells and Tsc2-null MEFs, FIG. 8D). Elevated ROS levels are well-known to occur in TSC-null cells (Finlay et al., 2003; Finlay et al., 2005), suggesting that THZ1 treatment further increases this level of ROS to a level causing apoptosis induction and cell death.

To examine this hypothesis further, the effects of antioxidants on cell death induction by THZ1 were examined. N-acetyl-cysteine (NAC), a ROS scavenger, restored ROS levels to near baseline in THZ1 treated TSC-null cells (FIG. 8E). Furthermore, NAC treatment significantly reduced the cell death seen in TSC-null cells in response to THZ1 treatment (FIG. 8F lower panel). In addition, to examine whether glutathione depletion was the proximate cause of apoptosis in THZ1-treated TSC-null cells, cells were treated with GSH reduced ethyl ester (GSH-MEE), a membrane-permeable derivative of GSH. GSH-MEE co-treatment rescued the viability of TSC-null cells treated with THZ1 (FIGS. 8F and 9C), indicating that glutathione depletion is a critical mechanism of THZ1-induced cell death.

### THZ1 Induces TSC-Dependent Cell Death Via Induction of Mitochondrial ROS

ROS generation occurs in multiple intracellular sites, including the cytosol, peroxisomes, plasma membrane, and ER. However, the majority of ROS is produced in the mitochondria when electrons escape from the mitochondrial respiratory chain and react with molecular oxygen (Trachootham et al., 2009; Venditti et al., 2013). The origin of the high ROS induced by THZ1 treatment was assessed by staining TSC-null cells treated with THZ1 for 16 hours with MitoSOX Red, which fluoresces red when oxidized by superoxide. The samples were counterstained with MitoTracker Green, which localizes to mitochondria regardless of mitochondrial membrane potential, and were then examined by confocal microscopy. THZ1 treatment of TSC-null cells caused an increase in mtROS compared with DMSO (FIG. 8G). Interestingly, the combination of rapamycin with THZ1 treatment showed a further increase in mtROS levels (FIG. 8G). Based on these observations overall but without wishing to be bound by theory, it was concluded that THZ1 induces TSC-dependent cell death via induction of elevated mitochondrial ROS in TSC-null cells.

### THZ1 Treatment of TSC Null Cells Leads to Major Reductions in Expression of Glutathione Biosynthesis Genes

Given the critical role of CDK7 in transcription through phosphorylation of the RNAPolII CTD (Akhtar et al., 2009; Drapkin et al., 1996; Glover-Cutter et al., 2009; Kwiatkowski et al., 2014), it was expected that THZ1 was affecting gene transcription via CDK7 inhibition. Previous studies cancer cells have shown that THZ1 treatment can cause selective loss of cancer-specific oncogene expression through both epigenetic silencing and transcriptional inhibition, leading to tumor cell death (Chipumuro et al., 2014; Christensen et al., 2014; Kwiatkowski et al., 2014; Wang et al., 2015; Zhang et al., 2017).

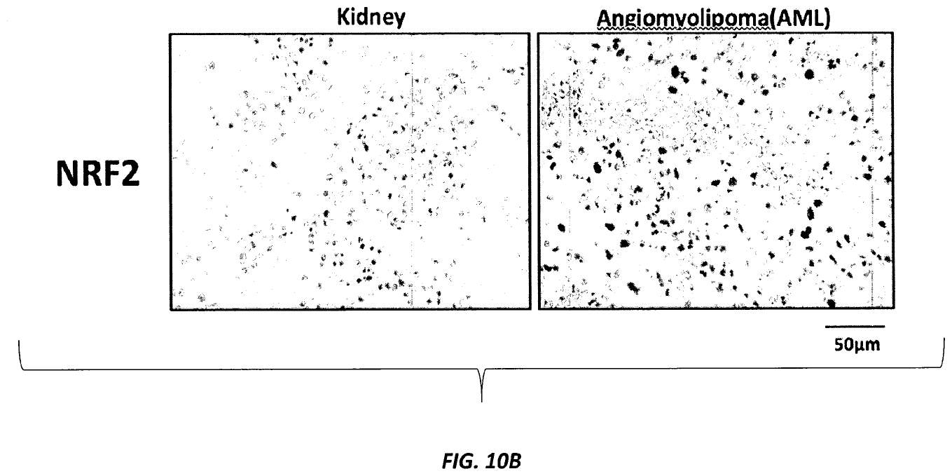

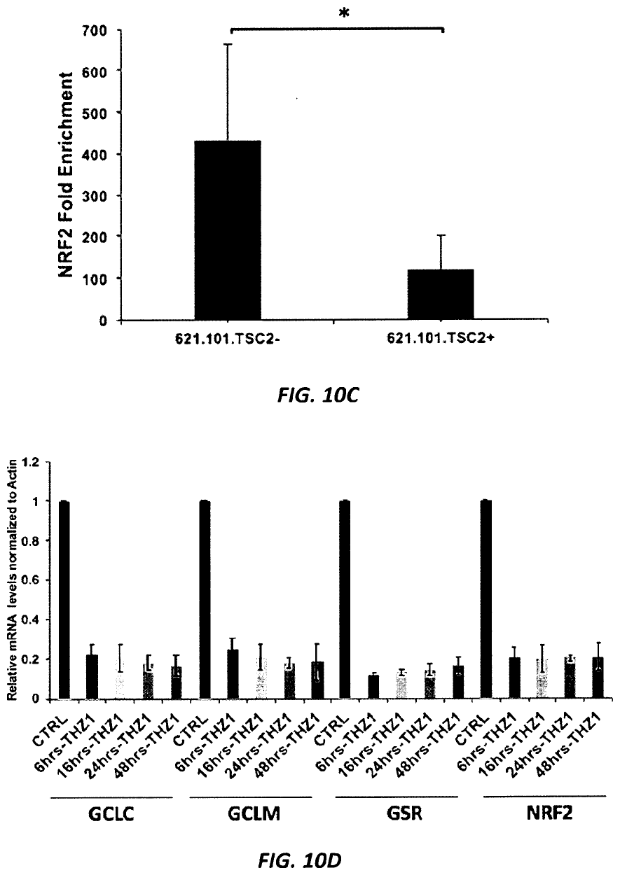

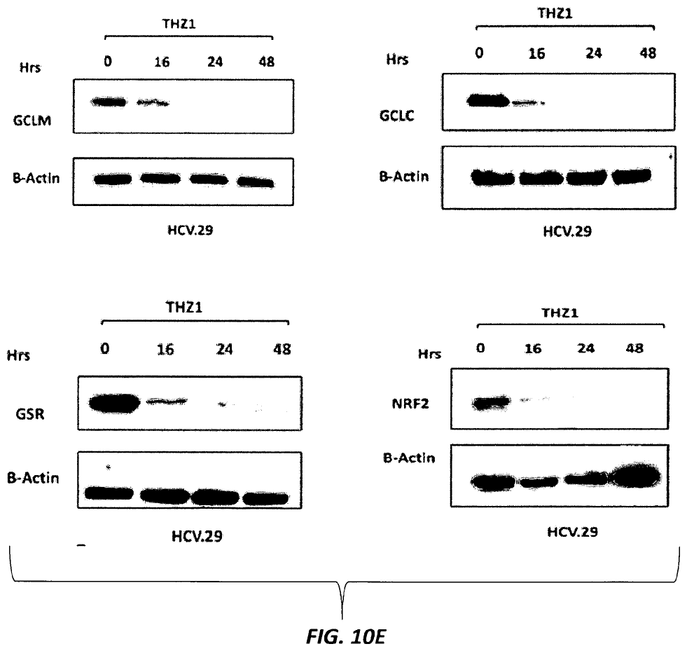

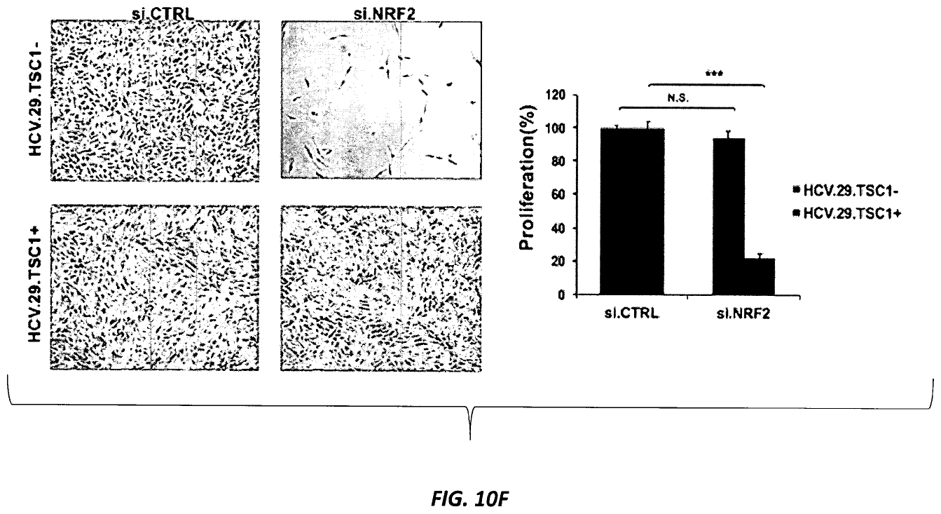

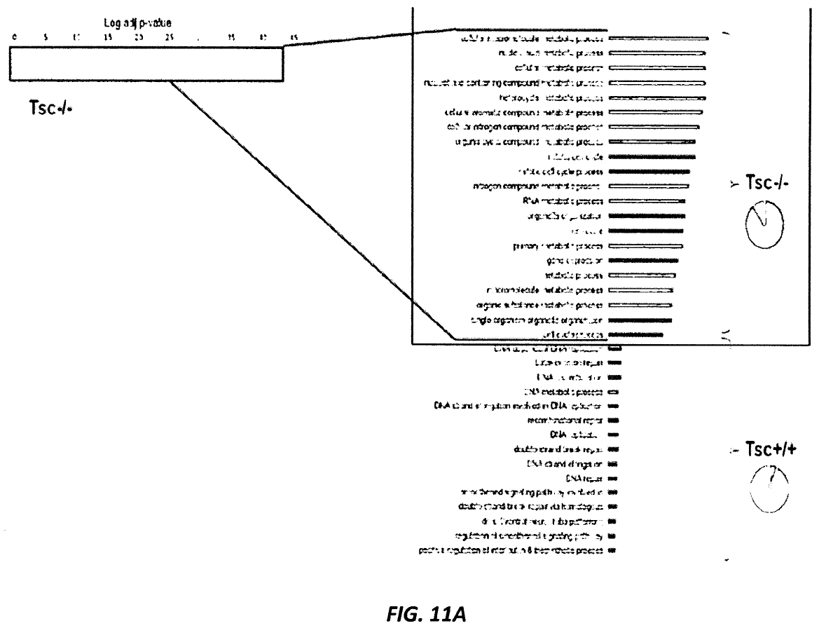

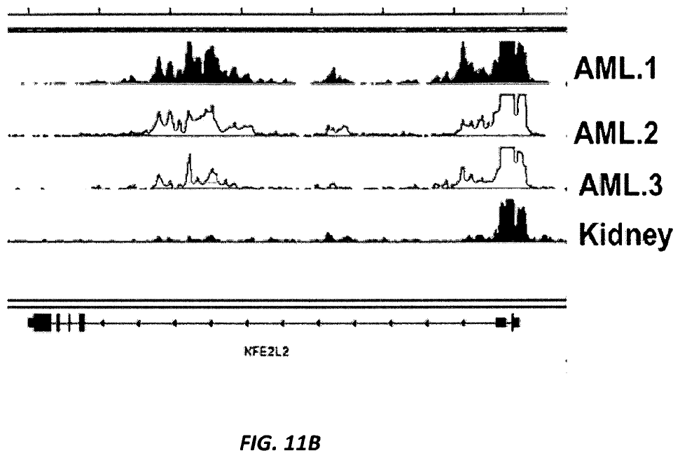

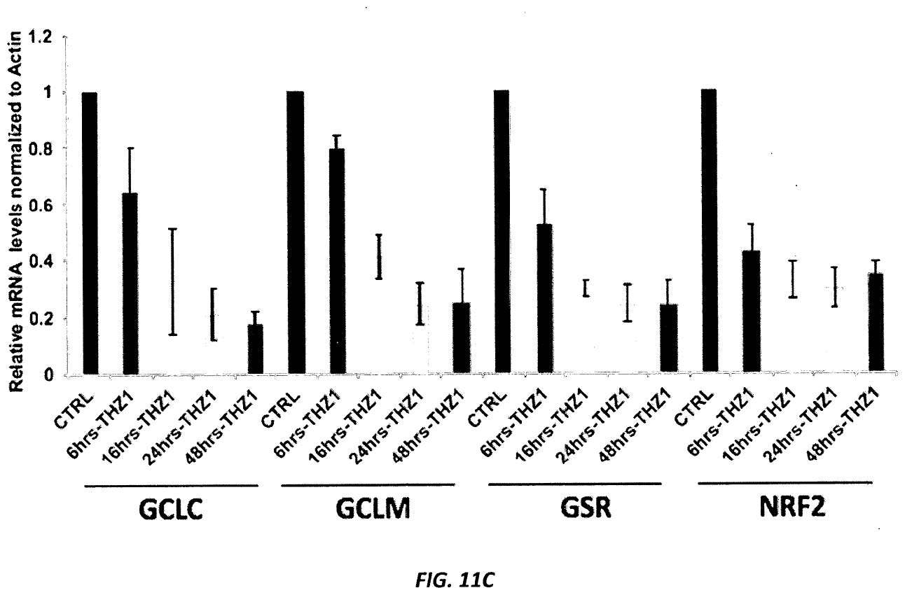

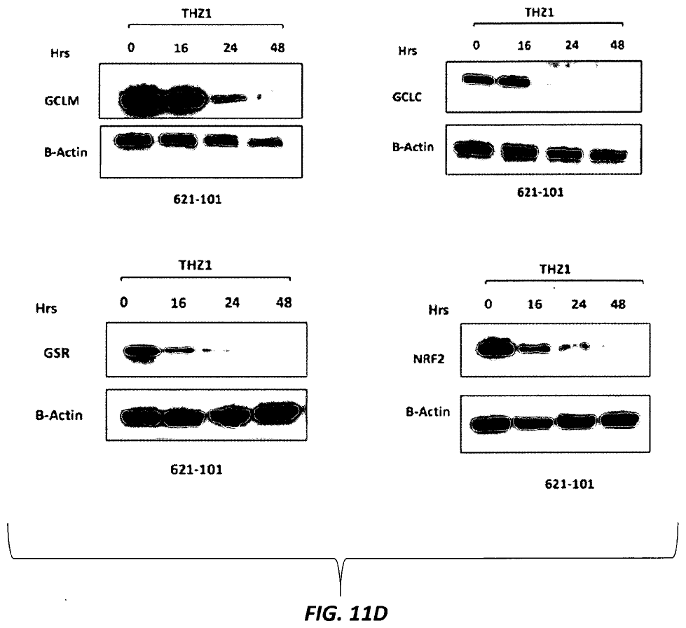

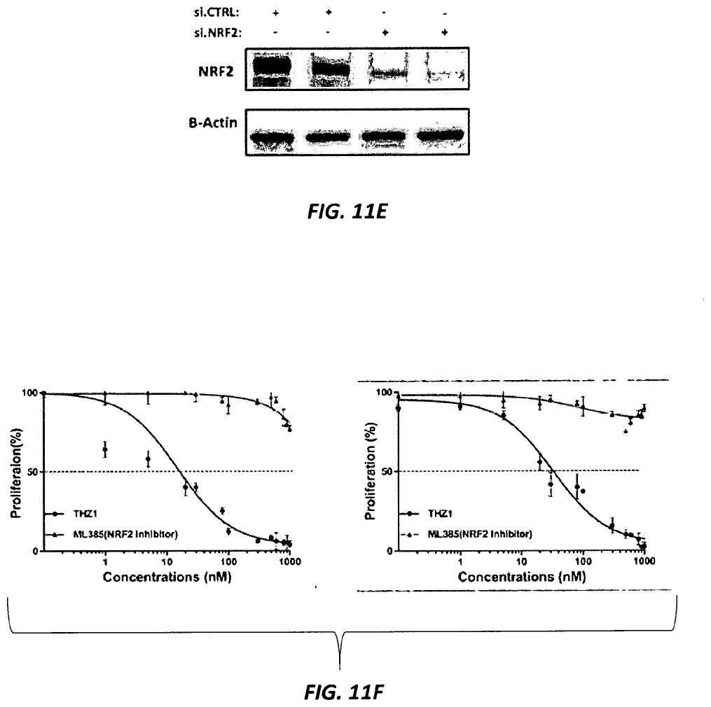

To examine global gene expression effects in TSC-deficient and TSC wild type cells, RNA-Seq was performed on TSC1− HCV.29 cells and TSC1-addback HCV.29 cells treated with 30 nM THZ1 for 6 hours. Many genes showed markedly different expression, with 1128 genes showing a >5-fold lower expression in THZ1-treated TSC1− HCV.29 cells compared with THZ1-treated TSC1-addback HCV.29 cells. In contrast 90% of those genes showed similar levels (<1.5-fold up or down) in the two cell lines untreated (FIG. 10A). Gene ontology analysis of the differentially-expressed genes in THZ1-treated TSC1-null cells compared with TSC1-addback cells showed that they were significantly enriched for metabolomic pathways (FIG. 11A). Interestingly, Nuclear factor-erythroid 2-related factor 2 (NFE2L2, also known as NRF2), which is a master regulator of antioxidant defense gene expression and has been shown to play a vital role in protecting cells from ROS (Tonelli et al., 2017) was reduced in expression in the THZ1-treated HCV.29-TSC1− cells. It was next asked whether THZ1 would induce similar gene expression changes in angiomyolipoma cells. Genome-wide expression data indicated was significantly downregulated upon THZ1 treatment in angiomyolipoma cells (data not shown).

The inventors next sought a mechanistic explanation for the sensitivity of NRF2 expression to THZ1 treatment and CDK7 inhibition. NRF2 was found to be highly marked with H3K27ac by ChIP-PCR analysis in the 621-101-TSC2− cell line, more so than was seen in the 621-101-TSC2+ addback cells (FIG. 10C). As above, previous reports have shown that super enhancer-marked genes are particularly sensitive to THZ1 inhibition of CDK7 (Chipumuro et al., 2014; Kwiatkowski et al., 2014; Loven et al., 2013). Therefore, in aggregate, these data indicate that CDK7 inhibition in TSC null cells targets NRF2 for epigenetic and transcriptional suppression, more so than is seen in parallel wild type cells.

NRF2 target genes are known to lead to cell-protective effects to reduce ROS, and include glutathione synthetic enzymes to enhance levels of glutathione (GSH) (Harvey et al., 2009; Hayes and Dinkova-Kostova, 2014). GSH is synthesized by the consecutive action of two enzymes, glutamate-cysteine ligase (GLC) and glutathione synthetase (GSS). GLC is composed of the glutamate-cysteine ligase catalytic subunit (GCLC) and the glutamate-cysteine ligase modifier subunit (GCLM), and is the rate-limiting enzyme for GSH synthesis. Glutathione exists in both reduced (GSH) and oxidized (GSSG) states, and is converted from GSH to GSSG by contact with ROS. GSH is regenerated from GSSG by the enzyme glutathione reductase (GSR). The expression of NRF2, GCLC, GCLM, and GSR are all coordinately reduced in response to THZ1 treatment in TSC null cells at both RNA (FIG. 10D, 11C) and protein levels (FIGS. 10E, 11D). At the protein level, these effects are dramatic with a >90% reduction in expression of each protein at 24 hours after THZ1 treatment (data not shown).

The most common TSC-related tumor, angiomyolipoma, also showed high level expression of NRF2, in comparison to normal kidney by immunohistochemistry (IHC) (FIG. 10B), indicating that NRF2 overexpression is a consistent response to TSC complex loss in vivo as well as in vitro.

Taken together, these observations indicate that NRF2-driven GSH biosynthetic gene expression is sensitive to THZ1 in TSC null cells as a result of their dependency on CDK7 for efficient transcription and downstream translation. Hence, without wishing to be bound by theory, these results indicate that THZ1-induced death of TSC-null cells is NRF2-dependent. To confirm this, siRNA against NRF2 was used to knockdown NRF2 expression in TSC deficient cells. NRF2 siRNA led to marked effects on cell proliferation in HVC29-TSC1− cells in contrast to HCV29-TSC1+ addback controls (FIGS. 10F, 11E), indicating that TSC null cells are more sensitive to NRF2 loss than wild type cells.

### THZ1 has Anti-Tumor Efficacy in Both Genetic and Xenograft Tumor Models of TSC

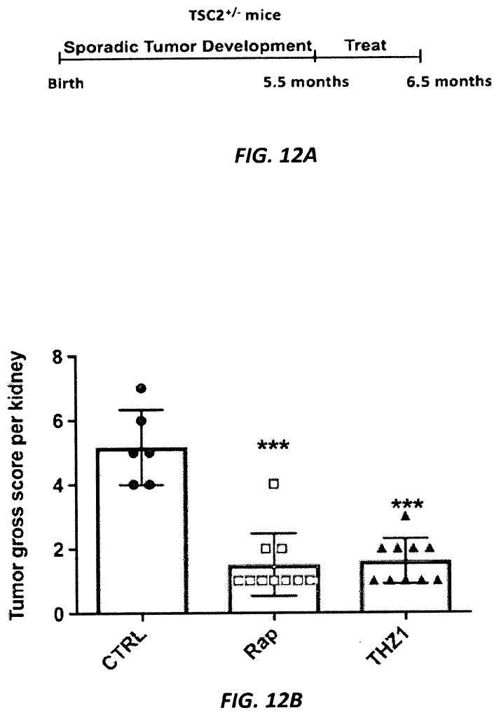

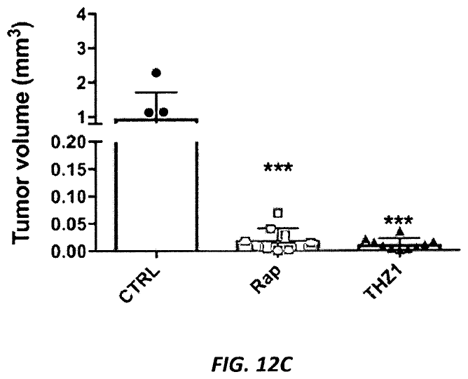

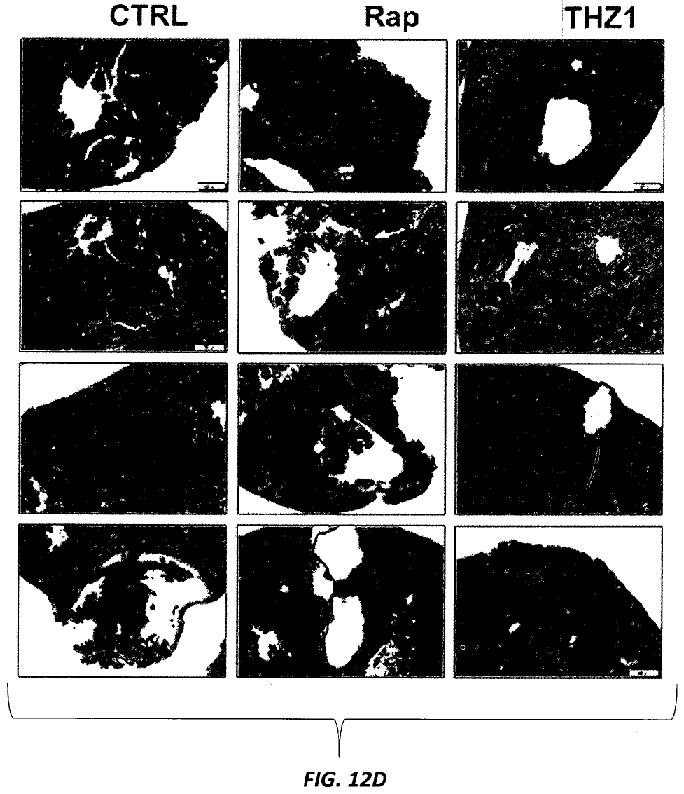

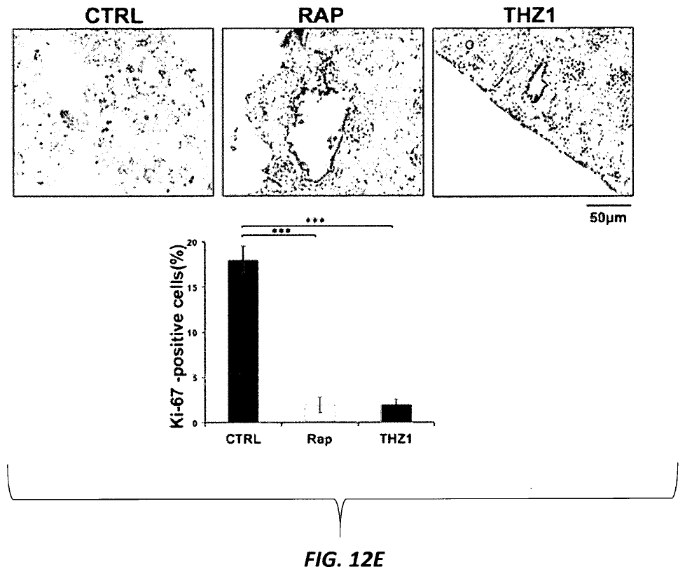

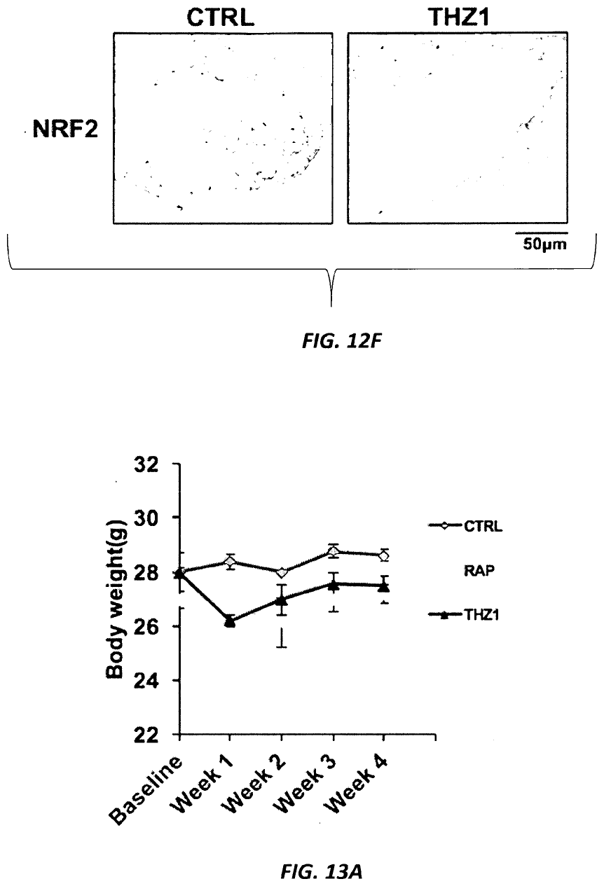

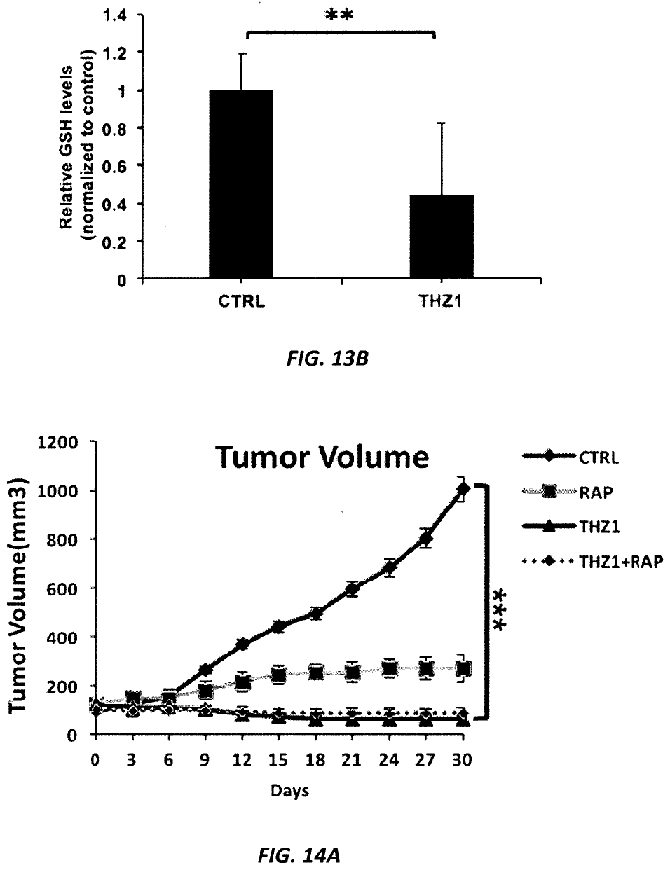

It was next asked whether THZ1 treatment can be effective for TSC-related tumor growth inhibition in vivo. Tsc2+/− A/J strain mice develop kidney cystadenomas by 4 months of age, and provide a native in vivo model that is genetically identical to human TSC patients, and is driven by spontaneous second hit events that lead to complete loss of Tsc2 expression and mTORC1 activation (Auricchio et al., 2012; Guo and Kwiatkowski, 2013; Woodrum et al., 2010). Tsc2+/− A/J mice were treated with vehicle, THZ1, or rapamycin for 1 month beginning at 5.5 months of age, when cystadenomas are well established in this model (FIG. 5A) (Auricchio et al., 2012; Guo and Kwiatkowski, 2013; Woodrum et al., 2010). THZ1 was administered by intraperitoneal injection of 10 mg/kg twice a day for 29 days (a standard dose, Wang et al., 2015), and rapamycin at 3 mg/kg 3 times per week. The mice tolerated this treatment well, without loss of body weight or other obvious effect (FIG. 13A). Rapamycin was dramatically effective in reducing tumor volume by about 99%, as assessed semi-quantitatively on H&E-stained sections (FIG. 12C), similar to what we have seen previously (Guo and Kwiatkowski, 2013). THZ1 showed effects similar to those of rapamycin in reducing tumor volume by about 99% (FIG. 12C). Consistent with this major response, there was a major difference in tumor histologic appearance with post-treatment kidney lesions consisting of cysts, with rare small papillary extensions into the cyst lumen. In contrast, papillary and solid adenoma lesions were seen in the vehicle treated mice. Ki67 staining, indicative of proliferation, was markedly reduced in the residual lesions seen after either rapamycin or THZ1 (FIG. 12E). In addition, cystadenoma cells comprising these lesions showed robust expression of NRF2 prior to, but not after treatment with THZ1, indicating inhibition of Nrf2 expression by THZ1 treatment (FIG. 12F). Furthermore, a significant reduction in total GSH levels was observed in THZ1-treated Tsc2+/− kidney tissue in comparison to vehicle control tumors (FIG. 13B).

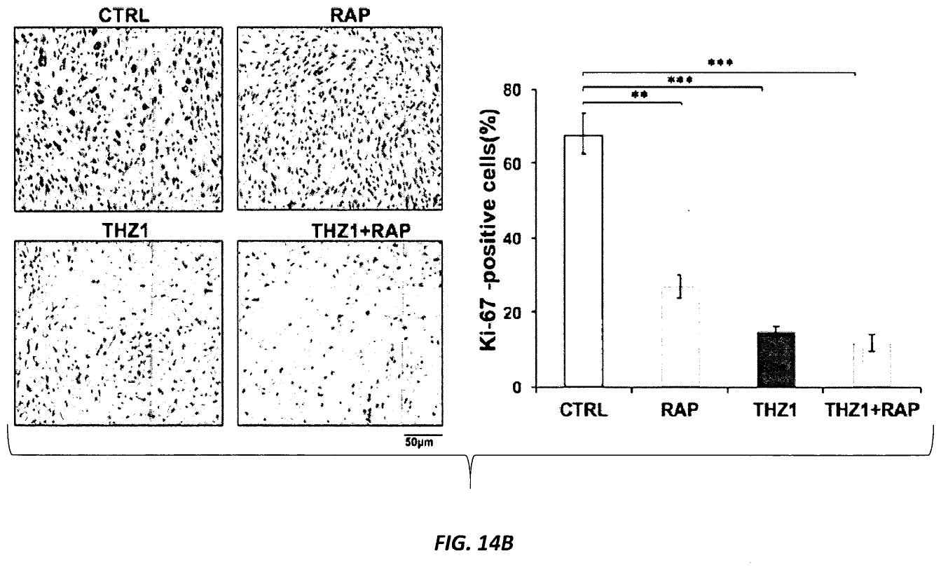

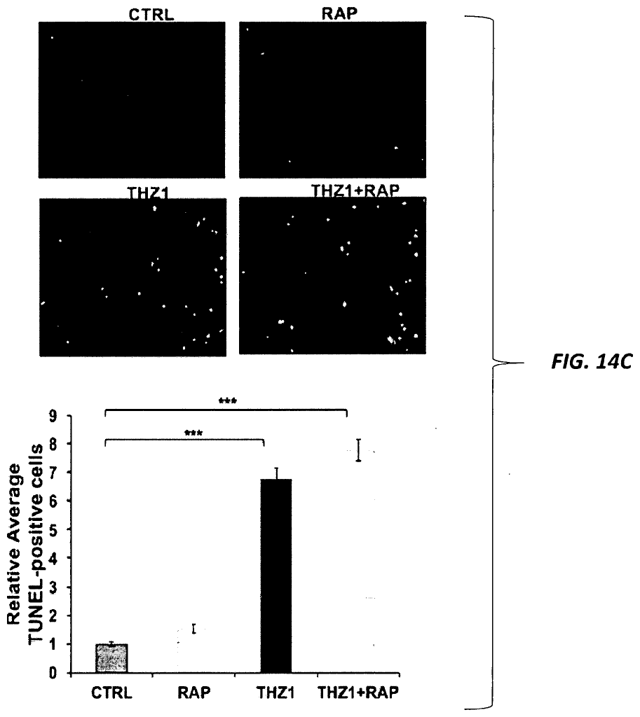

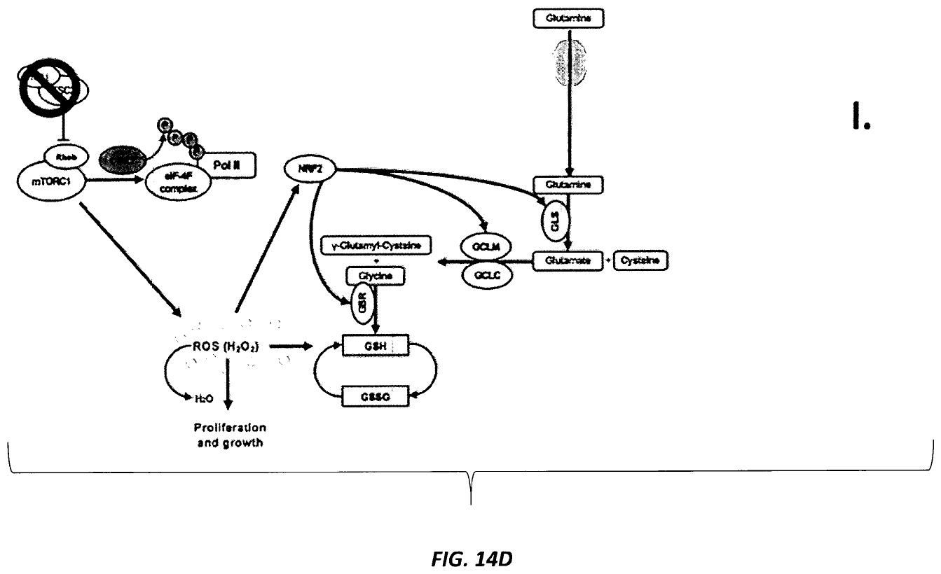

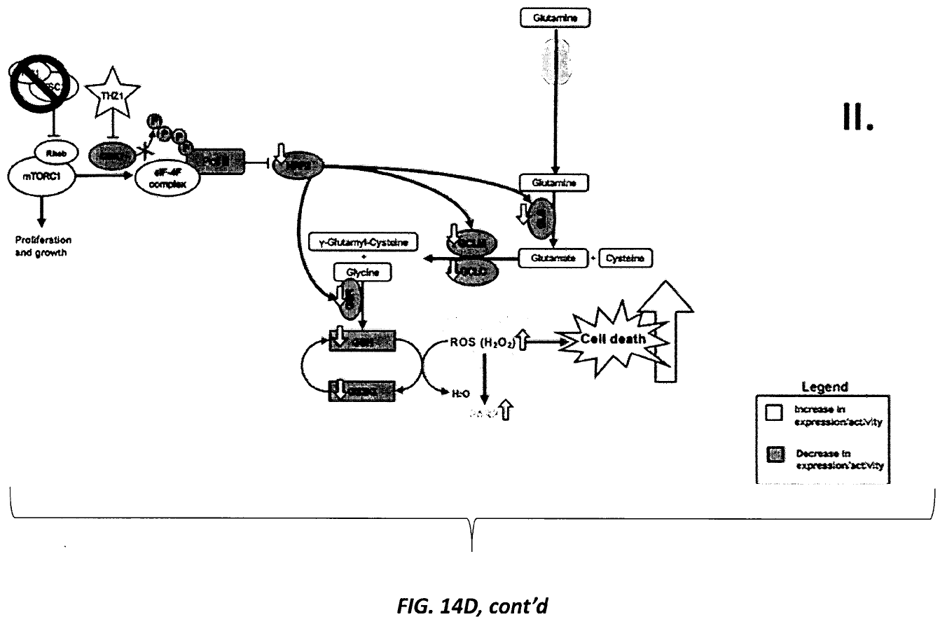

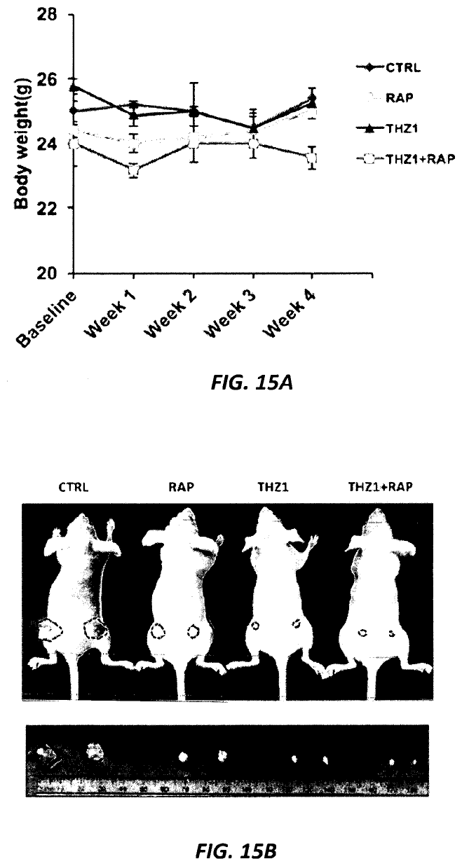

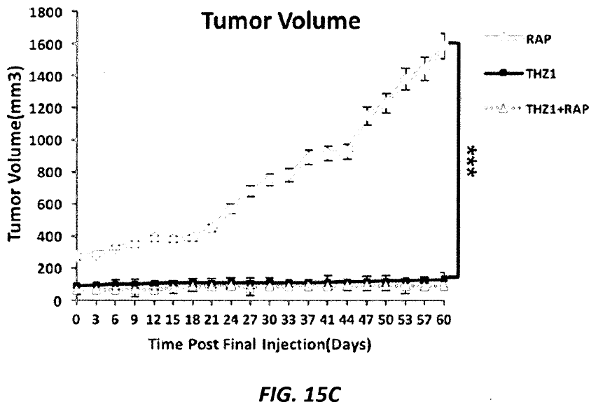

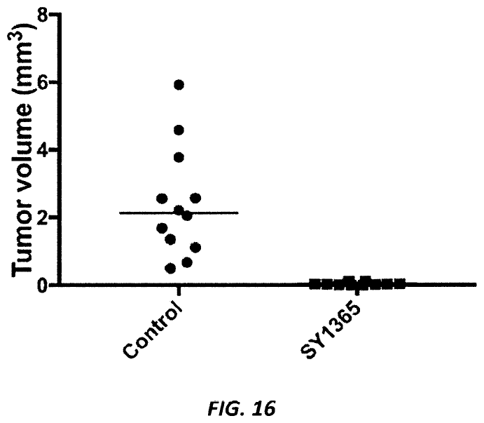

To validate these observations of in vivo efficacy of THZ1 for treatment of tumors with TSC complex loss, a xenograft model was used with the HVC29-TSC1− cell line. Xenografts were generated by standard subcutaneous injection, and mice were then randomized to treatment with either vehicle, THZ1(10 mg/kg IP twice a day), rapamycin (3 mg/kg IP 3 days per week), or both drugs initiated 4 weeks after flank injection of HCV.29 cells, when tumors first became palpable and measurable (FIG. 14A). No effect on body weight or other evidence of toxicity was observed (FIG. 15A).

Tumor volumes were measured by calipers. Both THZ1 alone and the combination led to a significant reduction in the size of the tumor nodules (FIGS. 14A and 15B). In contrast, mice treated with rapamycin alone showed a reduced growth rate, but did not show reduction in tumor size in comparison to the size at initiation of treatment. IHC assessment of Ki67 showed that all 3 treatments caused a reduction in proliferation rates, although this was greater for each of THZ1 and the combination in comparison to rapamycin (FIG. 14B). Furthermore, both THZ1 and the combination treatment led to persistent apoptotic cell death after 4 weeks, whereas this was not seen in the rapamycin or control groups (FIG. 14C). Finally, in a cohort of xenograft mice, the treatments were discontinued and tumor growth was observed without intervention after the one month of treatment. The rapamycin-only treated mice showed progressive tumor growth over 2 months until the end of this experiment. In contrast the THZ1 and combination treated mice showed no re-growth of subcutaneous tumors during that interval (FIG. 15C).

Taken together, these results indicate that in TSC− null cells THZ1-induced endogenous ROS inhibits NRF2 by transcriptional inhibition and GSH depletion ultimately leading to an energetic crisis and cell death (FIG. 14D).

### REFERENCES

Akhtar, M. S., Heidemann, M., Tietjen, J. R., Zhang, D. W., Chapman, R. D., Eick, D., and Ansari, A. Z. (2009). TFIIH kinase places bivalent marks on the carboxy-terminal domain of RNA polymerase II. Mol Cell 34, 387-393.
Auricchio, N., Malinowska, I., Shaw, R., Manning, B. D., and Kwiatkowski, D. J. (2012). Therapeutic trial of metformin and bortezomib in a mouse model of tuberous sclerosis complex (TSC). PloS one 7, e31900.
Catanzaro, E., Calcabrini, C., Turrini, E., Sestili, P., and Fimognari, C. (2017). Nrf2: a potential therapeutic target for naturally occurring anticancer drugs? Expert Opin Ther Targets 21, 781-793.
Chipumuro, E., Marco, E., Christensen, C. L., Kwiatkowski, N., Zhang, T., Hatheway, C. M., Abraham, B. J., Sharma, B., Yeung, C., Altabef, A., et al. (2014). CDK7 inhibition suppresses super-enhancer-linked oncogenic transcription in MYCN-driven cancer. Cell 159, 1126-1139.
Christensen, C. L., Kwiatkowski, N., Abraham, B. J., Carretero, J., Al-Shahrour, F., Zhang, T., Chipumuro, E., Herter-Sprie, G. S., Akbay, E. A., Altabef, A., et al. (2014). Targeting transcriptional addictions in small cell lung cancer with a covalent CDK7 inhibitor. Cancer Cell 26, 909-922.
Drapkin, R., Le Roy, G., Cho, H., Akoulitchev, S., and Reinberg, D. (1996). Human cyclin-dependent kinase-activating kinase exists in three distinct complexes. Proc Natl Acad Sci USA 93, 6488-6493.
Finlay, G. A., Hunter, D. S., Walker, C. L., Paulson, K. E., and Fanburg, B. L. (2003). Regulation of PDGF production and ERK activation by estrogen is associated with TSC2 gene expression. Am J Physiol Cell Physiol 285, C409-418.
Finlay, G. A., Thannickal, V. J., Fanburg, B. L., and Kwiatkowski, D. J. (2005). Platelet-derived growth factor-induced p 42/44 mitogen-activated protein kinase activation and cellular growth is mediated by reactive oxygen species in the absence of TSC2/tuberin. Cancer Res 65, 10881-10890.
Glover-Cutter, K., Larochelle, S., Erickson, B., Zhang, C., Shokat, K., Fisher, R. P., and Bentley, D. L. (2009). TFIIH-associated Cdk7 kinase functions in phosphorylation of C-terminal domain Ser7 residues, promoter-proximal pausing, and termination by RNA polymerase II. Mol Cell Biol 29, 5455-5464.
Guo, Y., and Kwiatkowski, D. J. (2013). Equivalent benefit of rapamycin and a potent mTOR ATP-competitive inhibitor, MLN0128 (INK128), in a mouse model of tuberous sclerosis. Molecular cancer research: MCR 11, 467-473.
Harvey, C. J., Thimmulappa, R. K., Singh, A., Blake, D. J., Ling, G., Wakabayashi, N., Fujii, J., Myers, A., and Biswal, S. (2009). Nrf2-regulated glutathione recycling independent of biosynthesis is critical for cell survival during oxidative stress. Free Radic Biol Med 46, 443-453.
Hayes, J. D., and Dinkova-Kostova, A. T. (2014). The Nrf2 regulatory network provides an interface between redox and intermediary metabolism. Trends Biochem Sci 39, 199-218.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Kwiatkowski, N., Zhang, T., Rahl, P. B., Abraham, B. J., Reddy, J., Ficarro, S. B., Dastur, A., Amzallag, A., Ramaswamy, S., Tesar, B., et al. (2014). Targeting transcription regulation in cancer with a covalent CDK7 inhibitor. Nature 511, 616-620.
Larochelle, S., Amat, R., Glover-Cutter, K., Sanso, M., Zhang, C., Allen, J. J., Shokat, K. M., Bentley, D. L., and Fisher, R. P. (2012). Cyclin-dependent kinase control of the initiation-to-elongation switch of RNA polymerase II. Nat Struct Mol Biol 19, 1108-1115.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Moinova, H. R., and Mulcahy, R. T. (1999). Up-regulation of the human gamma-glutamylcysteine synthetase regulatory subunit gene involves binding of Nrf-2 to an electrophile responsive element. Biochem Biophys Res Commun 261, 661-668.
Tonelli, C., Chio, I. I. C., and Tuveson, D. A. (2017). Transcriptional Regulation by Nrf2. Antioxid Redox Signal.
Trachootham, D., Alexandre, J., and Huang, P. (2009). Targeting cancer cells by ROS-mediated mechanisms: a radical therapeutic approach? Nat Rev Drug Discov 8, 579-591.
Venditti, P., Di Stefano, L., and Di Meo, S. (2013). Mitochondrial metabolism of reactive oxygen species. Mitochondrion 13, 71-82.
Wang, Y., Zhang, T., Kwiatkowski, N., Abraham, B. J., Lee, T. I., Xie, S., Yuzugullu, H., Von, T., Li, H., Lin, Z., et al. (2015). CDK7-dependent transcriptional addiction in triple-negative breast cancer. Cell 163, 174-186.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Wild, A. C., Moinova, H. R., and Mulcahy, R. T. (1999). Regulation of gamma-glutamylcysteine synthetase subunit gene expression by the transcription factor Nrf2. J Biol Chem 274, 33627-33636.
Woodrum, C., Nobil, A., and Dabora, S. L. (2010). Comparison of three rapamycin dosing schedules in A/J Tsc2+/− mice and improved survival with angiogenesis inhibitor or asparaginase treatment in mice with subcutaneous tuberous sclerosis related tumors. Journal of translational medicine 8, 14.
Yuan, M., Breitkopf, S. B., Yang, X., and Asara, J. M. (2012). A positive/negative ion-switching, targeted mass spectrometry-based metabolomics platform for bodily fluids, cells, and fresh and fixed tissue. Nat Protoc 7, 872-881.
Zarei, M., Lal, S., Parker, S. J., Nevler, A., Vaziri-Gohar, A., Dukleska, K., Mambelli-Lisboa, N.C., Moffat, C., Blanco, F. F., Chand, S. N., et al. (2017). Posttranscriptional Upregulation of IDH1 by HuR Establishes a Powerful Survival Phenotype in Pancreatic Cancer Cells. Cancer Res 77, 4460-4471.
Zhang, Z., Peng, H., Wang, X., Yin, X., Ma, P., Jing, Y., Cai, M. C., Liu, J., Zhang, M., Zhang, S., et al. (2017). Preclinical Efficacy and Molecular Mechanism of Targeting CDK7-Dependent Transcriptional Addiction in Ovarian Cancer. Mol Cancer Ther 16, 1739-1750.
Zhou, Q., Li, T., and Price, D. H. (2012). RNA polymerase II elongation control. Annu Rev Biochem 81, 119-143.

1. A method for treating tuberous sclerosis complex (TSC) in a subject, the method comprising: administering an inhibitor of cyclin dependent kinase 7 (CDK7) to a subject in need thereof, thereby treating tuberous sclerosis complex in the subject.

2. The method of claim 1, wherein the CDK7 inhibitor inhibits expression and/or activity of CDK7 by at least 10% compared to the expression and/or activity of CDK7 in a cell of the subject prior to treatment.

3. The method of claim 2, wherein the CDK7 inhibitor inhibits cell proliferation or viability preferentially in TSC1 and/or TSC2 deficient cells.

4. The method of claim 1, wherein the CDK7 inhibitor reduces aberrant cell proliferation in the subject.

5.-7. (canceled)

8. The method of claim 1, wherein the CDK7 inhibitor comprises THZ1 having the formula of Formula I, or a derivative thereof that retains CDK7 inhibition activity:

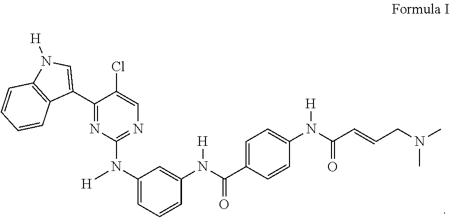 [SMILES: CN(C)C/C=C/C(=O)Nc1ccc(C(=O)Nc2cccc(Nc3ncc(Cl)c(-c4c[nH]c5ccccc45)n3)c2)cc1]

9. The method of claim 8, wherein the THZ1 derivative comprises SY-1365.

10. The method of claim 1, wherein the CDK7 inhibitor comprises CT7001 having the formula of Formula II:

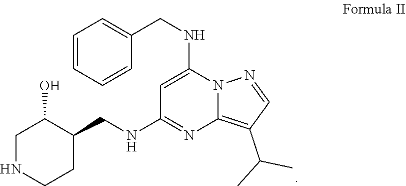 [SMILES: CC(C)c1cnn2c(NCc3ccccc3)cc(NC[C@H]3CCNC[C@@H]3O)nc12]

11. The method of claim 1 wherein the CDK7 inhibitor is selected from the group consisting of Compounds 1-186 of Table 1.

12. The method of claim 1, further comprising administering at least one additional agent.

13. The method of claim 12, wherein the at least one additional agent comprises rapamycin or an analog thereof that inhibits mTORC1 activity.

14. The method of claim 1, further comprising a step of detecting a genetic defect in TSC1 and/or TSC2 in the subject.

15. A pharmaceutical formulation comprising an amount of a CDK7 inhibitor effective to treat tuberous sclerosis complex in a subject in need thereof, and a pharmaceutically acceptable carrier.

16. (canceled)

17. The formulation of claim 15, wherein the CDK7 inhibitor comprises a molecule selected from: a) THZ1 of Formula I, or a derivative thereof that retains CDK7 inhibition activity: 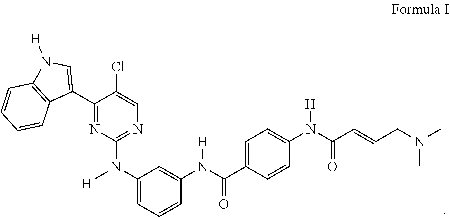 [SMILES: CN(C)C/C=C/C(=O)Nc1ccc(C(=O)Nc2cccc(Nc3ncc(Cl)c(-c4c[nH]c5ccccc45)n3)c2)cc1]
b) CT7001 having the formula of Formula II or a derivative thereof that retains CDK7 inhibition activity: 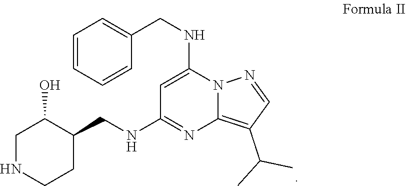 [SMILES: CC(C)c1cnn2c(NCc3ccccc3)cc(NC[C@H]3CCNC[C@@H]3O)nc12]
Or
c) a compound selected from the group consisting of compounds 1-186 of Table 1.

18. The formulation of claim 17, wherein the derivative of THZ1 that retains CDK7 inhibition activity comprises SY-1365.

19. The formulation of claim 13, further comprising a therapeutically effective amount of at least one additional therapeutic agent.

20. The formulation of claim 19, wherein the at least one additional therapeutic agent comprises rapamycin.

21.-26. (canceled)

27. A method for reducing growth and/or proliferation in a cell lacking TSC1 and/or TSC2, the method comprising: contacting a cell with an inhibitor of CDK7, thereby reducing the growth and/or proliferation of the cell.

28. A method for increasing apoptosis in a cell lacking TSC1 and/or TSC2, the method comprising: contacting a cell with an inhibitor of CDK7, thereby increasing apoptosis of the cell.

29. (canceled)
